SEQUENCE LISTING (26 sequences)

SEQ ID NO: 1 - DNA, 1782 bp, Homo sapiens
atgaaactacaggccgtgatggagacgctgttgcagcggcagcagcgggcgcgccaggagctggaggcccggcagcagctgccccccgatccccctgctgcaccccccggccgggcccgggctgcccccgacgaggacagagagcccgagagtgcccggatgcagcgggctcagatggccgcactggcagccatgcgggctgcagctgcgggcctgggacacccagccagccccggcggctctgaggatgggcccccaggctcggaggaggaggacgcggcccgggaggggacaccgggctcacccgggcgaggcagagaagggccaggagaggagcactttgaggacatggcctccgacgaggacatgaagcccaaatgggaggaggaggagatggaggaagacctcggggaggatgaggaggaggaggaggaggattacgaggatgaggaggaggaggaggacgaggaggggctgggccccccaggccctgccagcttgggcaccacggcactgttcccccgaaaggcccagccaccccaggccttccgcggcgatggcgttcccagggtgctggggggccaggagcggccggggcctggccctgcccaccccggaggggccgcccacgtagccccgcagctgcagccgcctgaccacggcgactggacttacgaggagcagtttaagcagctctacgaactcgacggggaccccaagaggaaggaattcctggatgacttgttcagcttcatgcagaagcgagggacacctgtgaaccgcatccccatcatggccaaacaggtccttgacctgttcatgctgtacgtgctggtgacggagaagggcggcctcgtggaggtcatcaacaagaagctgtggcgtgagatcaccaagggcctcaacctgcccacgtccatcaccagtgcagccttcaccctgcggacccaatacatgaagtacctgtacccctacgagtgtgagaagcggggcctcagtaaccccaatgagctccaggcagccatagacagcaaccgacgggagggccggcgccagagctttggtggctccctctttgcctactcgccaggcggggcacacggcatgctctcctcacccaagctacccgtgtcctccctgggcctggccgcaagcaccaatggcagctccatcacccccgcccctaagatcaagaaagaggaggactcagccatccccatcacagtccctggccgcctgcctgtgtccctggcgggccaccctgtggtggcagcccaggcagcagctgtgcaagcagcagccgcccaagcagctgtggccgcacaggcagctgccctggaacagctgcgggagaagctggagtctgcagagcctccggagaagaagatggccctggtggccgatgagcagcaacggctgatgcaacgtgcactccagcagaacttcctggccatggcggcccagctgcccatgagcattcggatcaacagccaagcctccgaaagccgccaggactctgctgtgaacctgacgggcaccaacggcagcaacagcatcagcatgtcggtggagatcaacggcatcatgtacacaggagttctgtttgctcagccgccggcccccacgccaacctctgctcccaacaaaggaggcggcggcggcggcggcagcagcagcaacgcaggcggccggggaggaaacaccggaaccagcggcggccaggctgggccagcggggctgtccacaccctccacatctacctcaaataactcgttgccttaa

SEQ ID NO: 2 - protein, 593 aa, Homo sapiens
MKLQAVMETLLQRQQRARQELEARQQLPPDPPAAPPGRARAAPDEDREPESARMQRAQMAALAAMRAAAAGLGHPASPGGSEDGPPGSEEEDAAREGTPGSPGRGREGPGEEHFEDMASDEDMKPKWEEEEMEEDLGEDEEEEEEDYEDEEEEEDEEGLGPPGPASLGTTALFPRKAQPPQAFRGDGVPRVLGGQERPGPGPAHPGGAAHVAPQLQPPDHGDWTYEEQFKQLYELDGDPKRKEFLDDLFSFMQKRGTPVNRIPIMAKQVLDLFMLYVLVTEKGGLVEVINKKLWREITKGLNLPTSITSAAFTLRTQYMKYLYPYECEKRGLSNPNELQAAIDSNRREGRRQSFGGSLFAYSPGGAHGMLSSPKLPVSSLGLAASTNGSSITPAPKIKKEEDSAIPITVPGRLPVSLAGHPVVAAQAAAVQAAAAQAAVAAQAAALEQLREKLESAEPPEKKMALVADEQQRLMQRALQQNFLAMAAQLPMSIRINSQASESRQDSAVNLTGTNGSNSISMSVEINGIMYTGVLFAQPPAPTPTSAPNKGGGGGGGSSSNAGGRGGNTGTSGGQAGPAGLSTPSTSTSNNSLP

SEQ ID NO: 3 - protein, 12 aa, Artificial Sequence
  Description of Artificial Sequence  Synthetic Peptide
GRGREGPGEEHE

SEQ ID NO: 4 - protein, 14 aa, Artificial Sequence
  Description of Artificial Sequence  Synthetic Peptide
ALHGSVLEGAGHAE

SEQ ID NO: 5 - protein, 12 aa, Artificial Sequence
  Description of Artificial Sequence  Synthetic Peptide
RTQYMKYLTPYE

SEQ ID NO: 6 - DNA, 46 bp, Artificial Sequence
  Description of Artificial Sequence  Synthetic Primer
taagtataaatatgtacatgagtacaccctccacttatttatctta

SEQ ID NO: 7 - DNA, 20 bp, Artificial Sequence
  Description of Artificial Sequence  Synthetic Primer
agctgcagccgcctgaccac

SEQ ID NO: 8 - DNA, 20 bp, Artificial Sequence
  Description of Artificial Sequence  Synthetic Primer
tgttgggagcagaggttggc

SEQ ID NO: 9 - DNA, 19 bp, Artificial Sequence
  Description of Artificial Sequence  Synthetic Primer
gtggcgtgagatcaccaag

SEQ ID NO: 10 - DNA, 19 bp, Artificial Sequence
  Description of Artificial Sequence  Synthetic Primer
cagaactcctgtgtacatg

SEQ ID NO: 11 - DNA, 21 bp, Artificial Sequence
  Description of Artificial Sequence  Synthetic Primer
caaaatccactacggaggcaa

SEQ ID NO: 12 - DNA, 16 bp, Artificial Sequence
  Description of Artificial Sequence  Synthetic Primer
tcccgtggtgggacga

SEQ ID NO: 13 - DNA, 22 bp, Artificial Sequence
  Description of Artificial Sequence  Synthetic Primer
atgtgcccattccagctgtcgc

SEQ ID NO: 14 - DNA, 20 bp, Artificial Sequence
  Description of Artificial Sequence  Synthetic Primer
tgtcctgagttccccaatgg

SEQ ID NO: 15 - DNA, 25 bp, Artificial Sequence
  Description of Artificial Sequence  Synthetic Primer
aaacccagtttaaccacatcttcat

SEQ ID NO: 16 - DNA, 32 bp, Artificial Sequence
  Description of Artificial Sequence  Synthetic Primer
cacattcagaaatcagcactcagtccttgtca

SEQ ID NO: 17 - protein, 7 aa, Artificial Sequence
  Description of Artificial Sequence  Synthetic Peptide
PKKKRKV

SEQ ID NO: 18 - DNA, 20 bp, Artificial Sequence
  Description of Artificial Sequence  Synthetic Primer
tgtcctgagttccccaatcc

SEQ ID NO: 19 - DNA, 25 bp, Artificial Sequence
  Description of Artificial Sequence  Synthetic Primer
aaacccagtttaaccacatcttcat

SEQ ID NO: 20 - DNA, 32 bp, Artificial Sequence
  Description of Artificial Sequence  Synthetic Primer
acaattcagaaatcagcactcagtccttgtca

SEQ ID NO: 21 - DNA, 21 bp, Artificial Sequence
  Description of Artificial Sequence  Synthetic Primer
ctagatccacatgtatgattt

SEQ ID NO: 22 - DNA, 21 bp, Artificial Sequence
  Description of Artificial Sequence  Synthetic Primer
gtctttcagacaatagattgg

SEQ ID NO: 23 - protein, 18 aa, Artificial Sequence
  Description of Artificial Sequence  Synthetic Peptide
GVLFAQPPAPTPTSAPNK

SEQ ID NO: 24 - protein, 25 aa, Artificial Sequence
  Description of Artificial Sequence  Synthetic Peptide
GGGGGGGSSSNAGGRGGNTGTSGGQ

SEQ ID NO: 25 - protein, 19 aa, Artificial Sequence
  Description of Artificial Sequence  Synthetic Peptide
AGPAGLSTPSTSTSNNSLP

SEQ ID NO: 26 - protein, 65 aa, Artificial Sequence
  Description of Artificial Sequence  Synthetic Peptide
GVLFAQPPPPTAPSAPGKGGVSSIGTNTTTGSRTGASGSTVSGGQVGLPGVSTPTMSSTSNNSLP

(19) United States
(12) Patent Application Publication
(10) Pub. No.: US 2005/0271651 A1
(43) Pub. Date: Dec. 8, 2005

(54) Inhibition of bright function as a treatment for excessive immunoglobulin production

(75) Inventor: Carol Webb, Oklahoma City, OK (US)

Correspondence Address: FULBRIGHT & JAWORSKI L.L.P., 600 CONGRESS AVE., SUITE 2400, AUSTIN, TX, 78701, US

(73) Assignee: Oklahoma Medical Research Foundation

(21) Appl. No.: 11/040,488

(22) Filed: Jan. 21, 2005

Related U.S. Application Data
(60) Provisional application No. 60/538,866, filed on Jan. 23, 2004.

(51) Int. Cl. A61K 38/17; A61K 48/00; A61K 39/395

(52) U.S. Cl. 424/131.1; 514/2; 514/44

(57) ABSTRACT

The present invention involves the identification of Bright as involved in immunoglobulin production, and the targeting of that function for the treatment of disease states associated with pathologic immunoglobulin production. Also provided are methods of identifying candidate substances with Bright-inhibitory activity.

The present application claims benefit of priority to U.S. Provisional Application Ser. No. 60/538,866, filed Jan. 23, 2004, the entire contents of which are hereby incorporated by reference.

### BACKGROUND OF THE INVENTION

1. Field of the Invention

The present invention relates generally to the fields of immunology and molecular biology. More particularly, it concerns inhibition of Bright function in the context of excessive or inappropriate immunogloblin production. Specifically, the invention relates to the use inhibitors of Bright in the treatment of disease, and to screening methods for finding inhibitors of Bright function.

2. Description of Related Art

Antibodies, also known as immunoglobulins (Ig), form a critical part of the human immune response. These large, bivalent receptor-like molecules, produced by B lymphocytes, are found both on cell surfaces and free in body fluids. Thanks to a complicated genetic system of gene rearrangement and somatic hypermutation, the human antibody repetoire is vast, with B cells capable of producing antibodies that bind to an almost endless array of selt and non-self antigens. In some cases, the binding of the antigen alone may be sufficient, impacting the ability of the antigen to perform its detrimental function. In other contexts, the antibodies mark the antigen for further removal or destruction by other immune cells (phagocytes, T-cells, etc.), or by the complement cascade.

Unfortunately, Ig production is not always beneficial. Numerous disease states characterized by excessive or inappopriate immunoglobulin production have been identified, including as systemic lupus erythematosus, rheumatoid arthritis, systemic sclerosis, polymyositis, Sjögren's Syndrome, graft rejection, Grave's disease, myasthenia gravis, cancer characterized by hyperimmunoglobulinemia, mononucleosis, and hyper-Ig syndromes. In many cases, the Ig produced attacks host cell antigens, causing inflammation and tissue destruction. Thus, it would be highly beneficial to identify mechanisms of down-regulating pathologic Ig production, and employing such methods as therapies for the aforementioned disease states.

### SUMMARY OF THE INVENTION

Thus, in accordance with the present invention, there is provided a method of suppressing immunoglobulin production in an activated B cell comprising contacting the cell with an inhibitor of Bright polypeptide function. The inhibitor may be an antisense molecule, an interfering RNA, or a ribozyme. The inhibitor may be a Bright-derived peptide, such as a peptide that comprises at least a portion of a Bright dimerization domain, at least a portion of a Bright DNA binding domain, or at least a portion of Btk-interacting domain. The inhibitor may also be a dominant-negative Bright polypeptide or an anti-Bright antibody or fragment thereof. The anti-Bright antibody or fragment thereof may be an F′ab, an humanized antibody, or a single chain antibody. The inhibitor may be delivered to the cell in a lipid delivery vehicle.

The inhibitor may also be a polypeptide or a nucleic acid, and the inhibitor may be delivered to the cell by an expression construct comprising an inhibitor coding region under the control of a promoter. The expression construct may be a viral expression vector, such as an adenoviral vector, an adeno-associated viral vector, a retroviral vector, a vaccinia viral vector, a herpesviral vector or a polyoma viral vector. The viral expression vector may be B cell tropic (e.g., Epstein Barr virus). The expression construct may also be a non-viral expression vector. The promoter may be an inducible promoter and the method may further comprise contacting the cell with an inducer of the promoter, such as a B cell specific promoter. The promoter may be a constitutive promoter.

In another embodiment, the present invention provides a method of treating a subject afflicted with disease state associated with excessive immunoglobulin production comprising administering to the subject an inhibitor of Bright polypeptide function. The inhibitor may be an antisense molecule, an interfering RNA, or a ribozyme. The inhibitor may be a Bright-derived peptide, such as a peptide that comprises at least a portion of a Bright dimerization domain, at least a portion of a Bright DNA binding domain, or at least a portion of Btk-interacting domain. The inhibitor may also be a dominant-negative Bright polypeptide or an anti-Bright antibody or fragment thereof. The anti-Bright antibody or fragment thereof may be an F′ab, an humanized antibody, or a single chain antibody. The inhibitor may be delivered to the cell in a lipid delivery vehicle.

The inhibitor may also be a polypeptide or a nucleic acid, and the inhibitor may be delivered to the cell by an expression construct comprising an inhibitor coding region under the control of a promoter. The expression construct may be a viral expression vector, such as an adenoviral vector, an adeno-associated viral vector, a retroviral vector, a vaccinia viral vector, a herpesviral vector or a polyoma viral vector. The viral expression vector may be B cell tropic (e.g., Epstein Barr virus). The expression construct may also be a non-viral expression vector. The promoter may be an inducible promoter and the method may further comprise contacting the cell with an inducer of the promoter, such as a B cell specific promoter. The promoter may be a constitutive promoter. The inhibitor may also be a peptide or a polypeptide which is fused to a TAT peptide or other transport of nuclear localizing peptide.

The method may further comprise administering to the subject an anti-inflammatory composition. The disease state may be selected from the group consisting of systemic lupus erythematosus, rheumatoid arthritis, systemic sclerosis, polymyositis, Sjögren's Syndrome, graft rejection, Grave's disease, myasthenia gravis, cancer characterized by hyperimmunoglobulinemia, mononucleosis, and a hyper-Ig syndrome. The inhibitor may be administered more than once, such as on a chronic basis.

In yet another embodiment, there is provided a method of screening for a suppressor of immunoglobulin production comprising (a) providing at least two Bright polypeptides; (b) contacting the Bright polypeptides with a candidate substance; and (c) assessing Bright dimer formation, wherein a decrease in Bright dimer formation, as compared to Bright dimer formation observed in the absence of the candidate substance, identifies the candidate substance as a suppressor of immunoglobulin production. The candidate substance may be a peptide, a non-functional Bright analog, an antibody or antibody fragment, or a small molecule organopharmaceutical.

In still yet another embodiment, there is provided a method of screening for a suppressor of immunoglobulin production comprising (a) providing at least one Bright polypeptide and one Btk polypeptide; (b) contacting the polypeptides with a candidate substance; and (c) assessing Bright interaction with Btk, wherein a decrease in Bright interaction with Btk, as compared to Bright interaction with Btk observed in the absence of the candidate substance, identifies the candidate substance as a suppressor of immunoglobulin production. The candidate substance may be a peptide, a non-functional Bright analog, an antibody or antibody fragment, or a small molecule organopharmaceutical.

In a further embodiment, there is provided a method of screening for a suppressor of immunoglobulin production comprising (a) providing at least one Bright polypeptide and one TFII-I polypeptide; (b) contacting the polypeptides with a candidate substance; and (c) assessing Bright interaction with TFII-I, wherein a decrease in Bright interaction with TFII-I, as compared to Bright interaction with TFII-I observed in the absence of the candidate substance, identifies the candidate substance as a suppressor of immunoglobulin production. The candidate substance may be a peptide, a non-functional Bright analog, an antibody or antibody fragment, or a small molecule organopharmaceutical.

In yet a further, embodiment, there is provided a method of screening for a suppressor of immunoglobulin production comprising (a) providing a recombinant cell that expresses Bright polypeptide and Btk polypeptide, and further comprises an immunoglobulin promoter linked to a screenable or selectable marker; (b) contacting the cell with a candidate substance; and (c) assessing expression of the marker, wherein a decrease in expression of the marker, as compared to marker expression observed in the absence of the candidate substance, identifies the candidate substance as a suppressor of immunoglobulin production. The recombinant cell may further express TFII-I.

As used herein the specification, “a” or “an” may mean one or more. As used herein in the claim(s), when used in conjunction with the word “comprising”, the words “a” or “an” may mean one or more than one. As used herein “another” may mean at least a second or more. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—Bright protein expression in human B cell lines is limited. (FIG. 1A) Western blots were performed using polyclonal rabbit anti-Bright with in vitro translated human and mouse Bright (lanes 1 and 2) and with 10 μg of nuclear extract from the fibroblast CHO cell line (last lane) and murine (BCg3R-1d) and human (CLO1) B cell lines that express endogenous Bright. Bright is indicated by the arrow. (FIG. 1B) Nuclear extracts from a panel of human cell lines (hematopoietic progenitor (K562), monocyte (U937), two T cell lines (Molt-4 and Jurkat), pre-B cell (697), mature B (Raji) and germinal center (CL01)) were analyzed for Bright protein expression by western blotting as in FIG. 1A. The same blot was developed with antibodies to lamin B. Data are representative of three experiments.

FIGS. 2A-D—Human Bright binds the prototypic DNA sequence motif, but Bright binding activity is not present in all B cell lines. (FIG. 2A) In vitro translated human Bright was bound to the bf150 Bright binding site in the presence or absence of anti-mouse peptide Bright, anti-human peptide Bright and preimmune goat serum in mobility shift assays. (FIG. 2B) Mobility shift assays were performed using the bf150 Bright binding site and 5 μg of nuclear extract prepared from a panel of cell lines. Two predominant complexes were observed in many of the cell lines and are labeled I and II. (FIG. 2C) Mobility shift assays were repeated in the presence of anti-human Bright antibody, antiserum to CDP, or preimmune control serum. Similar results were obtained when two additional anti-Bright reagents were used. The mouse B cell line, BCg3R-1d (BCg) was used as a positive control and the Bright protein complex in that cell line is indicated by the arrow. Note that additional protein complexes of the same apparent mobility did not react with anti-Bright sera and do not contain Bright protein. (FIG. 2D) Mobility shift assays were performed using nuclear extract from the 300212 cell line in the presence of 10-1000 molar excess of unlabeled competitor DNA. The specific inhibitor used was the double-stranded oligonucleotide containing the Bright binding site, while the nonspecific inhibitor contained mutations in the Bright binding sequence. Anti-human Bright antisera (Anti-HuBr) added to the last lane abrogated binding of the complex. Data are representative of a minimum of four independent experiments.

FIGS. 3A-B—Bright mRNA is expressed in normal human tissues. (FIG. 3A) A dot blot (Clontech) was hybridized with a 2 kb human Bright cDNA probe under stringent conditions (left panel). A labeled ubiquitin probe was used according to the manufacturer's instructions to demonstrate relative amounts of RNA from each tissue (right panel). This experiment was performed twice. (FIG. 3B) Relative expression of Bright in each tissue is shown after normalization for variation in total RNA levels with the ubiquitin probe.

FIG. 4A-B—Bright mRNA is expressed in early bone marrow B lineage subpopulations. RNA from sorted bone marrow early B lineage progenitor stem cell populations, pro- and pre-B cells, immature and recirculating B cells was subjected to RT-PCR with Bright-specific primers. Amplified products were detected by hybridization with a Bright cDNA probe spanning exons 1-8. In some cases, an additional smaller amplified product was observed and may represent an alternatively spliced product similar to that observed in mouse samples (Webb et al., 1989). Ethidium bromide stained gels show actin mRNA levels. Cell numbers of some subpopulations were so low that detection of the actin band with ethidium bromide was not possible. However, in these cases Bright was clearly evident. Data are representative of three separate sorting experiments.

FIGS. 5A-B—Bright is expressed in tonsil germinal center cells. (FIG. 5A) In situ hybridization of human tonsil tissues sections with antisense human Bright RNA (A and C) or control sense RNA (B and D) is shown. Darkly hybridizing oval-shaped germinal centers are apparent in A and in additional sections at higher magnification in C. Multiple sections of two separate tonsils gave similar results. (FIG. 5B) RNA from tonsil mononuclear cells sorted into naïve mantle (Bm1), founder (Bm2), dark zone centroblast (Bm3), light zone centrocyte (Bm4) and memory (Bm5) B cells was subjected to RT-PCR analyses using Bright and actin primers. Reactions without (RT⁻) and containing reverse transcriptase (RT⁺) were analyzed. Bright-hybridizing bands were identified as described in FIG. 4 and are indicated by arrows. Amplification of actin is shown by ethidium bromide staining. Actin RT⁻ lanes were negative. Data are representative of three sorting experiments.

FIG. 6—Bright mRNA is expressed at several discrete stages of B lymphocyte differentiation. A schematic representation of human B lymphocyte differentiation shows development from stem cells through memory cells. Surface markers used to distinguish among subpopulations are indicated and differentiation stages that express Bright mRNA are indicated with stars. Large stars indicate peak transcript expression levels.

FIG. 7—Btk associates with Bright in a subset of DNA-bound complexes. Mobility-shift assays were performed with nuclear extracts from two B cell lines (CL01 and 300212) and the T cell line, Molt-4. Anti-sera to CDP or the pleckstrin domain of Btk was added as in FIG. 2A-D. In some cases the anti-Btk sera was diluted 1:10 with PBS (0.1) prior to addition. Data are representative of six experiments.

FIG. 8—Bright protein domain structure and mutations. Schematic diagram of Bright showing domain structure and the targeted mutations.

FIGS. 9A-B—Effect of mutations on DNA binding activity. (FIG. 9A) Western blot analyses of in vitro translated protein mutants and wild-type (WT) Bright revealed proteins of the expected sizes. Nuclear extract from the cell line BCg3R-1d was used as a positive control. (FIG. 9B) EMSA analyses using the prototypic Bright binding site showed that only two mutants, REKLES and KIKK, maintained DNA binding activity. Data are representative of three experiments.

FIG. 10—Intracellular localization of Bright is not altered by mutation of Bright. Transfected CHO cells were stained with antibodies to Bright (red) and with the nuclear DAPI stain (blue) and were viewed using confocal microscopy. Fifty μm slices through the cell centers are shown.

FIGS. 11A-B—Tagged Bright proteins dimerize with native Bright. (FIG. 11A) Nuclear extracts from cells expressing Bright protein were subjected to size exclusion chromatography and protein fractions were analyzed for Bright by western blotting (lower panel). Data are presented graphically relative to molecular weight standards in the upper panel and are representative of four experiments. (FIG. 11B) C-terminal myc-his tagged Bright was either singly or coexpressed in CHO cells with native Bright. The top panel shows 10 μg of nuclear extract from each transfection developed with anti-Bright antibodies. Immunoprecipitations using anti-myc Ab (middle panel) and anti-Sp1 as an isotype control (bottom panel) were performed with each of the transfected extracts and developed with anti-Bright reagents. Arrows indicate immunoglobulin heavy chain cross-reactive with the secondary reagent. Data are representative of two separate experiments.

FIG. 12—ARID mutants interfere with native Bright DNA-binding activity. EMSA analyses of 3 ug of whole cell extract from transfected CHO cells were performed as in FIG. 9B. Cells were either singly transfected or cotransfected with a vector for native Bright expression. The bf150 probe alone is shown in the first lane of the second panel. Nuclear extract from the B cell line BCg3R-1d (BCg) was used as a positive control. Arrows indicate the Bright complex. Data are representative of three to four experiments.

FIG. 13—ARID mutants function as dominant negative proteins and interfere with wild-type Bright transcription activity. Real time PCR assays were performed using mRNA isolated from CHO cells transduced with DN Bright, wild-type Bright and a reporter gene containing the V1 S107 family heavy chain leader and first exon including 574 base pairs of promoter and 5′ flanking sequence with two previously characterized Bright binding sites (Novina et al., 1999). V1 expression was quantified using a standard curve. Data represent the average of three individual experiments where triplicate values were obtained.

FIG. 14—Endogenous Bright activity is altered with co-expression of mutant Bright. B cells (M12g3Ri) were transfected with tagged Bright, stimulated with LPS for 48 hours and sorted for GFP expression. Nuclear protein from untransfected, wild-type, and DM were analyzed by EMSA alone and in the presence of antibodies to Bright, myc, CDP or a Preimmune control. Bright and CDP are indicated by arrows. Data are representative of three separate experiments.

FIGS. 15A-C—Effect of dominant negative Bright on plasma cell markers. Splenic B cells were stimulated with LPS for 20 hours and then transduced with retrovirus containing dominant negative Bright for an additional 48-72 hours and sorted for GFP expression. (FIG. 15A) cDNA from these cells was analyzed by Real Time PCR for μ transcripts. The dominant negative Bright expressing cells exhibited little difference in μ expression versus cells expressing control vectors. (FIG. 15B) FACS analyses of surface expression of CD19 and CD138 on GFP-expressing transduced cells. The mean florescence intensity as well as the percent positive cells is indicated. Data are representative of two experiments. (FIG. 15C) Semi-quantitative RT-PCR of mRNA from transduced cells indicates relative expression levels of mRNAs. Actin expression was used as a control. Data shown represent three separate experiments.

FIGS. 16A-D—Bright activation of an immunoglobulin reporter gene requires Btk. (FIG. 16A) A standard curve for V1 DNA was generated by Real Time PCR. Each point reflects the average of triplicate CT values from four experiments. The y-intercept equation is shown. (FIG. 16B) Bright and Btk expression were assessed by western blotting in CHO cells transfected with Bright (Br), Btk and Br+Btk (lanes 3-5). Lane 1 contains extract from a control B cell line (Bcg3R-1d) and lane 2 contains extract from the CHO cell line transfected with control vectors. (FIG. 16C) V1 expression was averaged from triplicate samples from three experiments for CHO cells expressing Btk, Br, Br+Btk and control vectors using the standard curve in (FIG. 16A). Values were calculated using the formula y=−1.097Ln(x)+17.868 for each of the triplicate values and standard error of the mean (SEM) are shown. A diagram of the V1 reporter construct used is shown below. The primer (→ ←) and probe (•) locations are shown. Bright binding sites (x) are indicated. Exons are shown as boxes. (FIG. 16D) Extracts from (FIG. 16B) were assessed for Bright DNA-binding activity by EMSA with bf150. Lane numbers correspond to those shown in (FIG. 16B). F indicates free probe (first lane), and Bright complexes are indicated with an arrow.

FIGS. 17A-C—DNA-binding activity is necessary for Bright function as a transcription activator. (FIG. 17A) Average V1 expression was quantified in CHO cells transfected with double point mutant Bright (DPBr) and/or wild-type Bright plus Btk by Real Time PCR using triplicate samples from three individual experiments. Br+Br indicates cells were transfected with twice the amount of Bright vector DNA used with the Br/Btk transfectants. Data are expressed as percent activity with Br+Btk arbitrarily set at 100%. SEM bars are shown. (FIG. 17B) EMSA shows Bright binding complex (arrow) present in extracts from wild-type Bright (WT Br) and DPBr cells cotransfected with Btk. (FIG. 17C) Western blots show Bright and Btk expression levels in the transfected CHO cells.

FIG. 18—The distal Bright binding motif in the V1 promoter is necessary for Bright function. V1 deletion constructs containing zero (−125), one (−251) and two (−574) Bright binding sites were transfected with Br+Btk into CHO cells. V1 expression was measured using triplicate values from three Real Time PCR experiments. The average value from the full-length (−574) construct was set at 100% and the other values are presented as percent activity of that value. SEM bars are shown. Control transfected cells contained the full-length vector (−574) with negative control Bright and Btk plasmids.

FIGS. 19A-C—Functional Btk is required for Bright activity. (FIG. 19A) A schematic diagram depicts the pleckstrin (PH), tec (TH) and src (SH1-3) homology domains of wild-type Btk and the mutants used. R28C is the xid mutation; K430R renders Btk kinase inactive; and ΔPHTH lacks the pleckstrin and tec homology domains. (FIG. 19B) Western blots show expression of Bright (Br) and the Btk mutants in transfected CHO cell extracts. (FIG. 19C) V1 expression from the −574 full length promoter construct was measured in CHO cells transfected with wild-type Bright and either wild-type or mutant Btk by Real Time PCR as described in the previous figure legends. Each transfection was performed a minimum of three times and data were calculated from triplicate samples in each experiment. Average values for each transfection are presented as percent activity of the values obtained with wild-type Btk plus Bright that were set at 100%. SEM are shown. Control transfected cells contained the empty Btk vector, the Bright reverse orientation vector and the V1 reporter construct.

FIGS. 20A-C—Bright DNA-binding activity is facilitated by Btk and this enhanced binding requires the PHTH domain of Btk. (FIG. 20A) EMSAs were performed using bf150 and in vitro translated Bright (IVTBr) with or without the addition of exogenous recombinant wild-type Btk (rBtk). Unlabeled competitor DNA (100 molar excess) was added to samples in some lanes for 0 to 10 minutes before electrophoresis was begun. The Bright complex is indicated with an arrow. (FIG. 20B) Densitometric quantification of the bands in lanes 3-6 and 8-11 from (FIG. 20A) shows stabilization of Bright DNA-binding activity in the presence of Btk. (FIG. 20C) EMSAs were performed using in vitro translated Bright (IVT Br) or suboptimal levels (1:16 dilution, lanes 3-15) of IVT Bright with increasing amounts (triangles) of recombinant Btk. The arrow indicates the Bright complex. Probe alone is shown in the first lane. The last three lanes demonstrate the absence of binding activity produced by the maximum levels of each of the Btk proteins used in the absence of Bright. Expression of recombinant Btk proteins is shown by western blot in the boxed panel at the right. Data are representative of three experiments.

FIG. 21—Formation of Btk/Bright complexes is not dependent on Btk kinase activity or its PHTH domain, or the DNA-binding activity of Bright. Whole cell extracts from wild-type (Btk) or mutant (ΔPHTH, K430R) Btk and wild-type (Br) or DPBr Bright transfected CHO cells were immunoprecipitated with antibodies reactive with the myc tag on Bright (lanes 1-7) or with anti-Btk (lanes 8-14). Proteins were then immunoblotted with anti-Bright (top panels) or with anti-Btk antibodies (bottom panels). An unrelated antibody, anti-Sp1 (lanes 1 and 8) was used as a control for both the Btk and anti-myc monoclonals. In lanes 6, 7, 13 and 14, either Btk or Bright was expressed singly. Lanes 15-20 (Load) show levels of proteins prior to immunoprecipitation. The asterisk indicates the immunoprecipitated Ig heavy chain band.

FIGS. 22A-B—A third protein associates with Bright. Extracts from CHO cells transfected with myc-tagged Bright and/or Btk were immunoprecipitated with anti-myc antibody (lanes 2-5) or the isotype control, anti-Sp1 (lane 1) and immunoblotted (IB) with anti-Bright, anti-phosphotyrosine (FIG. 22A) or anti-BAP135/TFII-I antibodies (FIG. 22B). Positions of molecular weight markers are indicated. Data are representative of two separate experiments.

FIG. 23—Bright/Btk complexes bind Bright sites within a B cell line. Anti-Bright, anti-Btk or control goat antibodies (gtIg) were used in modified chromatin immunoprecipitation experiments with lysates of the B cell line, BCg3R-1d and the T cell hybridoma KD3B5.8. Immunoprecipitated DNA was PCR amplified at final dilutions of 1:100, 1:500 and 1:1000 (represented by triangles) for the presence of a Bright binding site (V1). Twenty percent of the DNA used for each immunoprecipitation was used as a positive control (Input). Data are representative of three experiments.

FIG. 24—Bright deletion constructs.

FIG. 25—Interaction of Bright and Bright deletion constructs with TFII-I.

FIG. 26—Human/Mouse Bright sequences necessary for TFII-I interactions. (SEQ ID NOS:23-26)

### DESCRIPTION OF ILLUSTRATIVE EMBODIMENTS

As discussed above, there is a great need for improved methods of treating autoimmune disorders, particularly those that are associated with pathologic immunoglobulin production. The inventor's previous studies indicated a link between Bright activity in the mouse and X-linked immunodeficiency disease (Webb et al., 2000). Because humans also suffer from X-linked immunodeficiency diseases, but have a much more severe phenotype than mice, they sought to determine whether human Bright was expressed in B lymphocytes and if it associated with Btk. In addition, previous analyses of human Bright had not been extended to the protein level (Kortschak et al., 1998). The data presented here demonstrate that Bright is not expressed in all human B lymphocyte subpopulations and show that Bright/Btk associated DNA-binding complexes exist in some human B cell lines. Human and mouse Bright expression and activities in nontransformed B cells were similar, indicating that human Bright shares important functions with the mouse protein, such as regulating immunoglobulin production.

I. Bright

The transcription factor Bright (B cell regulator of IgH transcription) is a member of a growing family of proteins that interact with DNA through a highly conserved A+T-rich interaction domain, or ARID (Herrscher et al., 1995). Currently, Bright is the only member of this family for which target sequences have been identified, and which binds to DNA in a sequence-specific fashion. ARID family proteins include the Drosophila proteins Dead ringer and eyelid that play important roles in lineage decisions in the gut and eyelid of the fruit fly, and are required for embryonic segmentation (Gregory et al., 1996; Treisman et al., 1997); retinoblastoma binding protein (Rbp1) that interacts with retinoblastoma protein in a cell cycle-specific fashion (Fattaey et al., 1993); and BDP, a ubiquitously expressed human protein identified in a two-hybrid screen as a novel protein that also interacts with retinoblastoma protein (Rb) (Numata et al., 1999). The yeast protein SWI/1 has homology to Bright, and is a component of a larger protein complex that serves to modulate chromatin organization in that organism (Peterson and Herskowitz, 1992; Burns and Peterson, 1997). Likewise, the human SWI-SNF complex contains a 270 kDa protein with non-sequence specific DNA binding activity that is also a member of the ARID family (Dallas et al., 2000). Thus, members of this family may participate in lineage decisions, cell cycle control, tumor suppression and modulation of chromatin. These functions are not mutually exclusive and may result from overlapping mechanisms.

Most ARID family proteins are expressed ubiquitously. However, murine Bright expression is largely limited to adult cells of the B lymphocyte lineage where its expression is tightly regulated and is restricted at the mRNA level to the pre-B cell and peanut agglutinin-high germinal center cell populations (Herrscher et al., 1995; Webb et al., 1991; Webb et al., 1998). Activated splenic B cells in the mouse can be induced to express Bright after antigen binding, but the protein is not present in the majority of peripheral IgM⁺ B cells (Webb et al., 1991; Webb et al., 1998). Induction of Bright expression in B cell lines or in mature activated B lymphocytes using lipopolysaccharide or antigen results in upregulation of IgH transcription approximately 3- to 6-fold above basal levels (Herrscher et al., 1995; Webb et al., 1991; Webb et al., 1989). Transcriptional activation is tightly associated with DNA binding sites 5′ of some V_{H} promoters or within the intronic Eμ enhancer.

Bright binding sites associated with the intronic Eμ enhancer also function as matrix-association regions, or MARs, A+T rich regions that have been proposed to organize chromatin into transcriptionally active domains (Herrscher et al. 1995; Webb et al., 1991). NFμNR (nuclear factor μ negative regulator) is another MAR-binding protein complex that binds DNA sequences overlapping Bright binding sites. NFμNR contains the ubiquitously expressed CAAAT displacement protein (CDP/Cut/Cux) (Wang et al., 1999). While non-B cells in the mouse express NFμNR, B lymphocytes generally do not exhibit such protein complexes. These data have led to the hypothesis that Bright and NFμNR play opposing roles in regulating the immunoglobulin locus (Webb et al., 1999). Transfection studies in which Bright and CDP were coexpressed showed repression of Bright (Wang et al., 1999). Therefore, Bright may activate transcription, directly or indirectly through chromatin remodeling or through more complex interactions with additional proteins. NFμNR may act in opposition to that activity (Wang et al., 1999).

The inventor recently determined that Bruton's tyrosine kinase, or Btk, associates with Bright in activated murine B lymphocytes (Webb et al., 2000). Btk is an X-linked gene that encodes a tyrosine kinase critical for proper development and maintenance of B lymphocytes both in humans and in mice (reviewed in (Conley et al., 1994; Satterthwaite and Witte, 1996). Defects in this enzyme account for 90% of the severe B cell immunodeficiencies in man, and result in X-linked agammaglobulinemia (XLA), an immunodeficiency state characterized by blocks at the pro-B cell stage of development and severely depressed serum antibody levels (Conley et al., 1994). Although Btk is clearly the defective gene product in both human and murine diseases, the molecular mechanisms by which Btk deficiencies result in blocks in B cell development are currently unknown. Of interest, X-linked immunodeficient (xid) mice, the mouse model for XLA, produce a mutated Btk protein that fails to form stable complexes with Bright (Webb et al., 2000). These data suggest that Bright may function as a component of the same signaling pathway(s) important in XLA.

Very little information is available regarding human Bright protein. Therefore, the inventor sought to characterize the human Bright homologue and to determine its expression in B lymphocyte subpopulations. Bright was cloned from a human B cell library and the sequence was determined to be identical to that published previously as Dril 1 (Kortschak et al., 1998). Although these studies suggested that Dril 1, or human Bright, mRNA was expressed in multiple tissues (Kortschak et al., 1998), protein and DNA binding activity were not investigated. The inventor's data indicate that Bright/Dril 1 mRNA may be expressed in a smaller number of tissues than previously thought. Furthermore, these data demonstrate that the human protein effectively binds the Bright prototype sequence motif. Investigation of sorted B cell subpopulations demonstrated that human Bright expression was similar in many ways to expression of the murine homologue; although, Bright mRNA was expressed at slightly earlier stages of normal B cell development in man than in the mouse. On the other hand, expression of Bright protein in human transformed cell lines differed dramatically from that observed in the mouse. Finally, results reveal that human Bright and Btk associate to form DNA-binding complexes, with which may further involve the Btk substrate TFII-I.

II. Peptides and Polypeptides

In certain embodiments, the present invention concerns Bright protein molecules. As used herein, a “protein” or “a polypeptide” generally refers, but is not limited to, a protein of greater than about 100 amino acids or the full length endogenous sequence translated form of a gene. A peptide is of from about 3 to about 100 amino acids. All the “proteinaceous” terms described above may be used interchangeably herein. A human Bright polypeptide sequence is provided in SEQ ID NO:2.

Proteins may be produced recombinantly or purified from natural sources. Shorter peptide molecules may be synthesized in solution or on a solid support in accordance with conventional techniques. Various automatic synthesizers are commercially available and can be used in accordance with known protocols. See, e.g., Stewart and Young (1984); Tam et al. (1983); Merrifield (1986); and Barany and Merrifield (1979), each incorporated herein by reference.

In certain embodiments the size of the at least one proteinaceous molecule may comprise, but is not limited to, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, and about 505 amino molecule residues, and any range derivable therein.

As used herein, an “amino acid” refers to any amino acid, amino acid derivitive or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

In certain embodiments, the proteinaceous composition comprises at least one protein, polypeptide or peptide. In further embodiments, the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (www.ncbi.nlm.nih.gov). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

Peptides may also be fused to other proteinaceous compositions, thereby altering or supplementing their properties. In a particular embodiment, a targeting moiety may be provided which facilitate cellular transport of the Bright derived peptide or polypeptide. In particular, sequences such as Tat can provide nuclear localization signals, thereby transporting peptides into the nucleus.

In certain embodiments, a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

III. Nucleic Acids

In certain embodiments of the present invention, nucleic acids derived from or encoding Bright, Btk and/or TFII-I are provided. In certain aspects, the nucleic acids may comprise wild-type or a mutant version of these genes. In particular aspects, the nucleic acid encodes for or comprises a transcribed nucleic acid. In other aspects, the nucleic acid comprises a nucleic acid segment of SEQ ID NO:1, or a biologically functional equivalent thereof. In particular aspects, the nucleic acid encodes a protein, polypeptide, peptide.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266 032, incorporated herein by reference, or via deoxynucleoside H-phosphonate intermediates as described by Froehler et al. (1986) and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of Bright. Thus, a “nucleic acid segment” may comprise any part of a gene sequence, of from about 2 nucleotides to the full length of Bright. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

The present invention also encompasses a nucleic acid that is complementary to a Bright-encoding nucleic acid. In particular embodiments the invention encompasses a nucleic acid or a nucleic acid segment complementary to the sequence set forth in SEQ ID NO:1. A nucleic acid is a “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

5. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present invention also concerns the isolation or creation of a recombinant construct or a recombinant host cell through the application of recombinant nucleic acid technology known to those of skill in the art or as described herein. A recombinant construct or host cell may comprise a Bright-encoding nucleic acid, and may express a Bright protein, peptide or peptide, or at least one biologically functional equivalent thereof.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Isolated substantially away from other coding sequences” means that the gene of interest forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engeneered or altered by the hand of man, and generally comprises one or more nucleic acid sequences organized by the hand of man.

In a non-limiting example, one or more nucleic acid constructs may be prepared that include a contiguous stretch of nucleotides identical to or complementary to SEQ ID NO:1. A nucleic acid construct may be about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc.; about 50,001, about 50,002, etc.; about 750,001, about 750,002, etc.; about 1,000,001, about 1,000,002, etc. Non-limiting examples of intermediate ranges include about 3 to about 32, about 150 to about 500,001, about 5,000 to about 15,000, about 20,000 to about 1,000,000, etc.

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of amino acids that are identical or functionally equivalent to the amino acids of SEQ ID NO:2 will be a sequence that is “essentially as set forth in SEQ ID NO:2,” provided the biological activity of the protein, polypeptide or peptide is maintained. Table 1 provides a listing of preferred human codons.

TABLE 1

| Amino Acids | | | Codons | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gln | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, the present invention also provides for nucleic acid sequences that have between about 70% and about 79%; or more preferably, between about 80% and about 89%; or even more particularly, between about 90% and about 99%; of nucleotides that are identical to the nucleotides of SEQ ID NO:1.

It will also be understood that this invention is not limited to the particular nucleic acid or amino acid sequence of SEQ ID NO:1 or 2. Recombinant vectors and isolated nucleic acid segments may therefore variously include these coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present invention encompass biologically functional equivalent proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide.

Encompassed by the invention are nucleic acid sequences encoding relatively small peptides or fusion peptides, such as, for example, peptides of from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length, or more preferably, of from about 15 to about 30 amino acids in length; as set forth in SEQ ID NO:2.

IV. Screening Methods

The present invention further comprises methods for identifying inhibitors of Bright activity that are useful in the prevention or treatment or reversal of pathologies associated with excessive antibody production. These assays may comprise random screening of large libraries of candidate substances; alternatively, the assays may be used to focus on particular classes of compounds selected with an eye towards structural attributes that are believed to make them more likely to inhibit the function of Bright.

To identify an inhibitor of Bright, one generally will determine the function of Bright in the presence and absence of the candidate substance. For example, a method generally comprises:
(a) providing an activated B cell that expresses Bright;
(b) contacting said with a candidate inhibitor substance; and
(c) measuring a Bright-related activity;
   wherein a decrease in a Bright related activity, as compared to Bright activity of an untreated cell, identifies the candidate substance as an inhibitor of Bright activity. Activities include stimulation of immunoglobulin production, Bright homodimerization, Bright interaction with Btk, or Bright interaction with TFII-I. Assays also may be conducted in isolated cells, cell extracts, organs, or in living organisms.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit the activity Bright. The candidate substance may be a protein or fragment thereof, a small molecule, or even a nucleic acid. It may prove to be the case that the most useful pharmacological compounds will be compounds that are structurally related to Bright, or a Bright interacting protein, such as Btk or TFII-I. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration, or which may affect the function of various other molecules. In one approach, one would generate a three-dimensional structure for a target molecule, or a fragment thereof. This could be accomplished by x-ray crystallography, computer modeling, or by a combination of both approaches.

It also is possible to use antibodies to ascertain the structure of a target compound, activator, or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecular libraries that are believed to meet the basic criteria for useful drugs in an effort to “brute force” the identification of useful compounds. Screening of such libraries, including combinatorially-generated libraries (e.g., peptide libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads. A common form of in vitro assay is a binding assay.

A technique for high throughput screening of compounds is described in WO 84/03564. Large numbers of small peptide test compounds are synthesized on a solid substrate, such as plastic pins or some other surface.

C. In Cyto Assays

The present invention also contemplates the screening of compounds for their ability to modulate Bright activity in cells. Various B cells and B cell lines can be utilized for such screening assays, including cells specifically engineered for this purpose. Other cells include spleen and bone marrow cells (murine and human), human cord blood cells and peripheral blood cells. Of particular interest are cells that contain an Ig promoter linked to a selectable or screenable marker gene.

D. In Vivo Assays

In vivo assays involve the use of various animal models, including transgenic animals that have been engineered to have specific defects or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria. Also, measuring toxicity and dose response can be performed in animals in a more meaningful fashion than in in vitro or in cyto assays.

V. Treating Diseases of Excessive Immunoglobulin Production

A. Disease States

Systemic lupus erythematosus (SLE). SLE is an autoimmune rheumatic disease characterized by deposition in tissues of autoantibodies and immune complexes leading to tissue injury (Kotzin, 1996). In contrast to autoimmune diseases such as MS and type 1 diabetes mellitus, SLE potentially involves multiple organ systems directly, and its clinical manifestations are diverse and variable (Reviewed by Kotzin and O'Dell, 1995). For example, some patients may demonstrate primarily skin rash and joint pain, show spontaneous remissions, and require little medication. At the other end of the spectrum are patients who demonstrate severe and progressive kidney involvement that requires therapy with high doses of steroids and cytotoxic drugs such as cyclophosphamide (Kotzin, 1996). More recently, treatment with rituximab (anti-CD2) has been attempted.

The serological hallmark of SLE, and the primary diagnostic test available, is elevated serum levels of IgG antibodies to constituents of the cell nucleus, such as double-stranded DNA (dsDNA), single-stranded DNA (ss-DNA), and chromatin. Among these autoantibodies, IgG anti-dsDNA antibodies play a major role in the development of lupus glomerulonephritis (Hahn and Tsao, 1993; Ohnishi et al., 1994). Glomerulonephritis is a serious condition in which the capillary walls of the kidney's blood purifying glomeruli become thickened by accretions on the epithelial side of glomerular basement membranes. The disease is often chronic and progressive and may lead to eventual renal failure.

The mechanisms by which autoantibodies are induced in these autoimmune diseases remains unclear. As there has been no known cause of SLE, to which diagnosis and/or treatment could be directed, treatment has been directed to suppressing immune responses, for example with macrolide antibiotics, rather than to an underlying cause. (e.g., U.S. Pat. No. 4,843,092).

Rheumatoid arthritis (RA). The exact etiology of RA remains unknown, but the first signs of joint disease appear in the synovial lining layer, with proliferation of synovial fibroblasts and their attachment to the articular surface at the joint margin (Lipsky, 1998). Subsequently, macrophages, T cells and other inflammatory cells are recruited into the joint, where they produce a number of mediators, including the cytokines interleukin-1 (IL-1), which contributes to the chronic sequelae leading to bone and cartilage destruction, and tumour necrosis factor (TNF-α), which plays a role in inflammation (Dinarello, 1998; Arend and Dayer, 1995; van den Berg, 2001). The concentration of IL-1 in plasma is significantly higher in patients with RA than in healthy individuals and, notably, plasma IL-1 levels correlate with RA disease activity (Eastgate et al., 1988). Moreover, synovial fluid levels of IL-1 are correlated with various radiographic and histologic features of RA (Kahle et al., 1992; Rooney et al., 1990).

In normal joints, the effects of these and other proinflammatory cytokines are balanced by a variety of anti-inflammatory cytokines and regulatory factors (Burger and Dayer, 1995). The significance of this cytokine balance is illustrated in juvenile RA patients, who have cyclical increases in fever throughout the day (Prieur et al., 1987). After each peak in fever, a factor that blocks the effects of IL-1 is found in serum and urine. This factor has been isolated, cloned and identified as IL-1 receptor antagonist (IL-1Ra), a member of the IL-1 gene family (Hannum et al., 1990). IL-1Ra, as its name indicates, is a natural receptor antagonist that competes with IL-1 for binding to type I IL-1 receptors and, as a result, blocks the effects of IL-1 (Arend et al., 1998). A 10- to 100-fold excess of IL-1Ra may be needed to block IL-1 effectively; however, synovial cells isolated from patients with RA do not appear to produce enough IL-Ira to counteract the effects of IL-1 (Firestein et al., 1994; Fujikawa et al., 1995).

Systemic sclerosis. Systemic sclerosis (SSc) is a connective tissue disease of unknown etiology characterized by fibrotic changes of the skin, subcutaneous tissue, and viscera; abnormalities of the microvasculature; and immune dysfunction. The literature has referred to the skin changes as “keloid” in nature. In early national surveys from the '60's, the incidence of SSc was reported to be 12 cases per 1 million population annually. More recent studies report a higher prevalence of SSc, on the order of 19-75 case per 100,000 population.

SSc can affect a wide variety of organs and tissues including the skin, gastrointestinal tract, lungs, heart, kidneys, and musculoskeletal system. Altered connective tissue metabolism characterized by increased deposition of extracellular matrix components like collagen, fibronectin and glycosaminoglycans has been observed in SSc. Lymphokines such IL-2, IL-4, and IL-6 were found in the sera of patients with scleroderma, but not in healthy control subjects. Activated T cells and/or antibody-dependent complement cascade likely stimulate the release of endothelial cytokines with subsequent endothelial damage, which facilitates adhesion and migration T cells and monocytes.

Polymyositis. Polymyositis is an inflammatory muscle disease that causes varying degrees of decreased muscle power. The disease has a gradual onset and generally begins late in the second decade of life. The primary symptom is muscle weakness, usually affecting those muscles that are closest to the trunk of the body (proximal). Eventually, patients have difficulty rising from a sitting position, climbing stairs, lifting objects, or reaching overhead. In some cases, distal muscles (those not close to the trunk of the body) may also be affected later in the course of the disease. Trouble with swallowing (dysphagia) may occur. The disease may be associated with other collagen vascular, autoimmune or infectious disorders. Treatment for generally consists of prednisone or immunosuppressants such as azathioprine and methotrexate.

Sjögren's syndrome. Sjögren's syndrome is a systemic autoimmune disease in which the body's immune system mistakenly attacks its own moisture producing glands. Sjögren's is one of the most prevalent autoimmune disorders, striking as many as 4,000,000 Americans, with 90% of patients being women. The average age of onset is late 40's although Sjögren's occurs in all age groups in both women and men.

About 50% of the time Sjögren's syndrome occurs alone, and 50% of the time it occurs in the presence of another connective tissue disease. The four most common diagnoses that co-exsist with Sjögren's syndrome are Rheumatoid Arthritis, Systemic Lupus, Systemic Sclerosis (scleroderma) and Polymyositis/Dermatomyositis. Sometimes researchers refer to these situations as “Secondary Sjögren's.”

Sjögren's is characterized by dry eyes and dry mouth, and may also cause dryness of other organs such as the kidneys, GI tract, blood vessels, lung, liver, pancreas, and the central nervous system. Many patients experience debilitating fatigue and joint pain. Symptoms can plateau, worsen, or go into remission. While some people experience mild symptoms, others suffer debilitating symptoms that greatly impair their quality of life.

Graft rejection. Tissue and organ grafts, though powerful tools for treating disease and injury, provoke powerful immune responses that can result in rapid graft rejection in the absence of immunosuppressive therapy. Pioneering studies conducted in the '40s and '50s established that allograft rejection was due to immune responses, later linked to T-lymphocytes. Specific immunological effector mechanisms responsible for graft rejection include cytotoxic T-cells, delayed-type hypersensitivity and antibody-dependent effects. Allograft-directed responses target cell-surface molecules called ‘human leucocyte antigens’ (HLAs), were first described by Dausset in 1958. HLAs are highly polymorphic and play a special role in immune recognition.

Grave's disease. Marked by nervousness and overstimulation, Grave's disease is the result of an overactive thyroid gland (hyperthyroidism). Thyroid hormones regulate metabolism and body temperature, and are essential for normal growth and fertility. But in excessive amounts, they can lead to the burn-out seen in this relatively common form of thyroid disease. It is unclear what triggers this problem, but the immune system is involved. In Grave's disease patients, they find antibodies specifically designed to stimulate the thyroid.

Along with nervousness and increased activity, Grave's disease patients may suffer a fast heartbeat, fatigue, moist skin, increased sensitivity to heat, shakiness, anxiety, increased appetite, weight loss, and sleep difficulties. They also have at least one of the following: an enlargement of the thyroid gland (goiter), bulging eyes, or raised areas of skin over the shins.

In many cases, drugs that reduce thyroid output are sufficient to control the condition. A short course of treatment with radioactive iodine, which dramatically reduces the activity of the thyroid, is another option for people past their childbearing years. In some cases, surgery to remove all or part of the thyroid (thyroidectomy) is needed. Surgery can also relieve some of the symptoms of Grave's disease. Bulging eyes, for example, can be corrected by creating enough extra space in the nearby sinus cavity to allow the eye to settle into a more normal position.

Myasthenia gravis. The number of myasthenia gravis patient in the United States alone is estimated at 0.014% of the population, or approximately 36,000 cases; however, myasthenia gravis is likely under diagnosed. Previously, women appeared to be more often affected than men, with the most common age at onset being the second and third decades in women, and the seventh and eighth decades in men. As the population ages, the average age at onset has increased correspondingly, and now males are more often affected than females, and the onset of symptoms is usually after age 50.

Patients complain of specific muscle weakness and not of generalized fatigue. Ocular motor disturbances, ptosis or diplopia, are the initial symptom of myasthenia gravis in two-thirds of patients. Oropharyngeal muscle weakness, difficulty chewing, swallowing, or talking, is the initial symptom in one-sixth of patients, and limb weakness in only 10%. Initial weakness is rarely limited to single muscle groups such as neck or finger extensors or hip flexors. The severity of weakness fluctuates during the day, usually being least severe in the morning and worse as the day progresses, especially after prolonged use of affected muscles. The course of disease is variable but usually progressive, resulting in permanent muscle weakness. Factors that worsen myasthenic symptoms are emotional upset, systemic illness (especially viral respiratory infections), hypothyroidism or hyperthyroidism, pregnancy, the menstrual cycle, drugs affecting neuromuscular transmission, and increases in body temperature.

In acquired myasthenia gravis, post-synaptic muscle membranes are distorted and simplified, having lost their normal folded shape. The concentration of ACh receptors on the muscle end-plate membrane is reduced, and antibodies are attached to the membrane. ACh is released normally, but its effect on the post-synaptic membrane is reduced. The post-junctional membrane is less sensitive to applied ACh, and the probability that any nerve impulse will cause a muscle action potential is reduced.

Cancer. The present invention also contemplates the overexpression of Bright in various cancers such as chronic lymphocytic leukemia, plasmacytomas and myelomas. In particular, cancers characterized by hyperimmunoglobulinemia may be treated.

Mononucleosis. Infectious mononucleosis, or “glandular fever,” is caused by the Epstein-Barr virus. Though usually not serious, splenic rupture is possible second to enlargement of the spleen. Like most herpesviruses, EBV will go latent in neural ganglia after ther active infective subsides. Some people with mono have minimal symptoms, such as fatigue, fever, sore throat and headache. Reports of chronic, sub-acute infection exist. One exposed, most people will develop immunity and will not be reinfected. One notable characteristic, and the basis for the disease name, is the presence of an elevated white blood cell count. In severe forms of the disease, hyper-IgM production is observed

Hyper-IgM syndrome. Patients with X-linked hyper-IgM (XHIGM) syndrome have a defect or deficiency in CD40 ligand, a protein that is found on the surface of T-lymphocytes. CD40 ligand is made by a gene on the X chromosome. Thus, this primary immunodeficiency disease is inherited as an X-linked recessive trait, and usually found only in boys. As a consequence of their deficiency in CD40 ligand, affected patients' T-lymphocytes are unable to instruct B-lymphocytes to switch their production of gammaglobulins from IgM to IgG and IgA. Patients with this primary immunodeficiency disease have decreased levels of serum IgG and IgA and normal or elevated levels of IgM. In addition, since CD40 ligand is important to other functions of T-lymphocytes, they also have a defect in some of the protective functions of their T-lymphocytes. Other forms of autosomal recessive Hyper-IgM syndrome have been discovered, but the responsible mutations have not yet been identified.

Hyper-IgD syndrome. The syndrome is typified by a very early age at onset (median, 0.5 years) and life-long persistence of periodic fever. Characteristically, attacks occur every 4-8 weeks and continue for 3-7 days, but the individual variation is large. Attacks feature high spiking fever, preceded by chills in 76% of patients. Lymphadenopathy is commonly present (94% of patients). During attacks, 72% of patients complain of abdominal pains, 56% of vomiting, 82% of diarrhea, and 52% of headache. Joint involvement is common in the hyper-IgD syndrome with polyarthralgia in 80% and a non-destructive arthritis, mainly of the large joints (knee and ankle), in 68% of patients.

Hyper-IgE syndrome. Hyper-IgE syndrome (HIES) is a primary immunodeficiency disease characterized by recurrent infections and marked immunoglobulin IgE elevation.

B. Pharmaceutical Inhibitors

Virtually any organopharmaceutical compound may produce the desired effect. Such compounds may be identified according to the screening methods described above.

C. Antisense Constructs

An alternative approach to inhibiting Bright is antisense. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Another general class of inhibitors is ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

E. RNAi

RNA interference (also referred to as “RNA-mediated interference” or RNAi) is another mechanism by which protein expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. See U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

F. Antibodies

In certain aspects of the invention, antibodies may find use as inhibitors of Bright. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

G. Peptides

Peptides may prove to be useful inhibitors of Bright function by competiting with or mimicking Bright domains that bind or interact with DNA, Btk, TFII-I or other molecules. Bright-derived peptides are therefore a particular type of compound that may prove useful in inhibiting Bright function.

Peptides may be produced by cleavage of polypeptides, such as Bright, with proteolytic enzymes (trypsin, chymotrypsin, etc.), or chemicals. Peptides may also be produced synthetically, using either recombinant techniques or chemical synthesis. The peptides may be designed around an existing structure, i.e., portions of Bright, or they may be selected for function from a randomized library.

H. Dominant Negative Bright

Dominant negative proteins are defective proteins with can negate the effects of normal, functional proteins when both are present in the same environment. In many cases, dominant negative proteins homo-multimerize and are thus able to “poison” a complext that contains one or more functional proteins. Dominant negative forms of Bright have been produce which act in just this manner. In designing dominant negative Bright molecules, several regions present useful points for mutation. First, changes in the DNA binding domain (ARID) that block DNA binding should produce dominant negative effects. Second, alterations in the nuclear localization sequence which block nuclear translocation should also result in a dominant negative form of Bright. Third, manipulation of the interaction domain may also cause a dominant negative function.

I. Combined Therapy

In another embodiment, it is envisioned to use an inhibitor of Bright function in combination with other therapeutic modalities. Thus, in addition to the therapies described above, one may also provide to the patient more “standard” hyperimmune therapies. Examples of other therapies include anti-inflammatory compounds.

Combinations may be achieved by contacting cells with a single composition or pharmacological formulation that includes both agents, or by contacting the cell with two distinct compositions or formulations, at the same time. Alternatively, the therapy using an inhibitor of Bright may precede or follow administration of the other agent(s) by intervals ranging from minutes to weeks. In embodiments where the agents are applied separately to the cell, one would generally ensure that a significant period of time did not expire between the time of each delivery, such that the agents would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an inhibitor or the other agent will be desired. In this regard, various combinations may be employed. By way of illustration, where the inhibitor of Bright is “A” and the other agent is “B,” the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated.

J. Therapeutic Agents

Pharmacological therapeutic agents and methods of administration, dosages, etc., are well known to those of skill in the art (see for example, the “Physicians Desk Reference,” Goodman and Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108, 4,166,452, and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxypropylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

VI. Vectors for Cloning, Gene Transfer and Expression

Within certain embodiments, expression vectors are employed to express various products including Bright, peptides, antibodies or fragments thereof, antisense molecules, ribozymes or interfering RNAs. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retro viruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse mammary tumor virus) | Glucocorticoids | Huang et al., 1981; Lee et |
| | | al., 1981; Majors et al., |
| | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis Factor | PMA | Hensel et al., 1989 |
| Thyroid Stimulating Hormone α Gene | Thyroid Hormone | Chatterjee et al., 1989 |

Of particular interest are promoters that are selectively active in B cells. A particular promoter in this group is the CD19 promoter (Maas et al., 1999).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. Delivery of Expression Vectors

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

Epstein-Barr virus, frequently referred to as EBV, is a member of the herpesvirus family and one of the most common human viruses. The virus occurs worldwide, and most people become infected with EBV sometime during their lives. In the United States, as many as 95% of adults between 35 and 40 years of age have been infected. When infection with EBV occurs during adolescence or young adulthood, it causes infectious mononucleosis 35% to 50% of the time. EBV vectors have been used to efficiently deliver DNA sequences to cells, in particular, to B lymphocytes. Robertson et al. (1986) provides a review of EBV as a gene therapy vector.

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

VII. Preparing Antibodies Reactive with or Inhibitory to Bright

In yet another aspect, the present invention contemplates an antibody that is immunoreactive or inhibitory to Bright, or any portion thereof. An antibody can be a polyclonal or a monoclonal antibody, it can be humanized, single chain, or even an Fab fragment. In a preferred embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, goats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to Bright epitopes.

In general, both polyclonal, monoclonal, and single-chain antibodies against Bright may be used in a variety of embodiments. A particularly useful application of such antibodies is in purifying native or recombinant Bright, for example, using an antibody affinity column. The operation of all accepted immunological techniques will be known to those of skill in the art in light of the present disclosure.

Means for preparing and characterizing antibodies are well known in the art (see, e.g., Harlow and Lane (1988), incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified PKD protein, polypeptide or peptide or cell expressing high levels of PKD. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al. (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### VIII. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cloning and antibody preparation. Plaque hybridization and phage DNA preparation were performed essentially as described (Webb et al., 1989). To obtain the human Bright sequence expressed in B lymphocytes, a human B lymphocyte cDNA library (Clontech, Palo Alto, Calif.) was screened with a 2 kB mouse Bright cDNA probe (Herrscher et al., 1995). Three unique clones hybridized with the mouse probe. Each of these shared the same 5′ Eco RI site resulting from library construction and was homologous to murine Bright from amino acid 238 through the 3′ untranslated sequence. The longest clone contained 1.1 kb of 3′ untranslated sequence. Kortschak et al. (1998), cloned and published sequences identical to that of the inventor and called this protein Dril 1, for homology to the Drosophila protein, DRI (SEQ ID NO:2). A full length clone was amplified from the CL01 B cell line by RT-PCR (SEQ ID NO:1). All sequences were confirmed using the OMRF sequencing core facility and were analyzed with Vector NTI software. In vitro translated proteins were produced with TNT rabbit reticulocyte lysates (Promega, Madison, Wis.).

Polyclonal anti-human Bright peptide sera and anti-mouse peptide sera were prepared by immunizing goats with MAP-peptides containing the human amino acid sequence GRGREGPGEEHE (SEQ ID NO:3) from the amino terminal domain and the corresponding mouse sequence from the analogous region ALHGSVLEGAGHAE (SEQ ID NO:4). Additional sera common to the ARID peptide RTQYMKYLTPYE (SEQ ID NO:5) from both mouse and human were also produced. Sera were tested for specific reactivity with the immunizing peptide by enzyme-linked immunosorbent assay and were affinity purified over peptide columns. Preimmune sera were collected for controls.

Western blotting. Proteins were subjected to SDS polyacrylamide gel electrophoresis under standard denaturing conditions in 7.5% acrylamide, and were transferred to nitrocellulose membranes and blocked in 0.5% gelatin and 0.05% thimerosol (Webb et al., 2000). Bright was detected with polyclonal rabbit anti-Bright and alkaline phosphatase labeled goat anti-rabbit immunoglobulin (Southern Biotech, Birmingham, Ala.). Lamin B was detected with a chicken antibody and goat anti-chicken secondary reagent (Santa Cruz Biologicals). Alkaline phosphatase-conjugated rabbit anti-goat IgG (Southern Biotech) or horse-radish peroxidase-conjugated rabbit anti mouse IgG were used as secondary reagents. Secondary reagents were preadsorbed against nuclear extracts from the cell line BCg3R-1d to decrease background. Phosphatase substrate was purchased from BioRad, and SuperSIgnal West Pico chemiluminescent substrate was purchased from Pierce (Rockford, Ill.).

Cell lines and tissue preparation. Transformed B cell lines representing different stages of differentiation were used and included: Nalm 16 (pro-B), 697 (pre-B), CL01 (mature B, Burkitt lymphoma, generous gift of P. Casali, Cornell U., N.Y.), BL2 (mature B, EBV negative) Ramos (mature B, EBV negative, Burkitt lymphoma), Daudi and Raji (mature B, EBV positive, Burkitt lymphoma), and 300212 (EBV-transformed peripheral blood cell line). Other cell lines used were: Jurkat and Molt-4 (T lymphoblasts), K562 (multipotential progenitor), U937 (myelomonocyte) and HeLa (epithelial cell line). All cells were grown in RPMI 1640 supplemented with 7% heat inactivated fetal calf serum (FCS), 2 mM L-glutamine, 100 U/ml penicillin, 100 μg/ml streptomycin, 5×10⁻⁵ M 2-ME and 1 mM sodium pyruvate. Phorbol myristate acetate (PMA) was suspended in dimethyl sulfoxide and used at a final concentration of 10 μM.

Bone marrow, tonsil and thymus were procured post-surgically from children and adults with non-hematologic disorders. Blood samples were obtained from adult volunteers. Umbilical cord blood was obtained post-delivery from the placentae of normal, full-term infants. All studies were performed in accordance with institutional review board regulations for the use of human subjects. Mononuclear cells from bone marrow, cord blood and peripheral blood lymphocytes (PBLs) were >95% pure after standard Ficoll-Hypaque density-gradient centrifugation (Medina et al., 2001). Adherent cells were removed by incubation at 37° C. for one hour, and mononuclear cell suspensions were resuspended in fresh media for further separation or for protein extraction. Single cell suspensions were prepared from tonsil as previously described (Pascual et al., 1994), and from fresh surgical thymus by mechanical disruption followed by filtration through wool columns and two successive Ficoll-Hypaque gradients.

In situ hybridization was performed with the Roche non-radioactive In Situ Hybridization kit, according to the manufacturer's directions. Briefly, fresh tonsils were serially sectioned, fixed and hybridized with labeled antisense or sense strand RNAs containing exons 3 through 8 of human Bright with 830 bases of 3′ untranslated region. After extensive washing, the sections were developed and examined microscopically. Three to four consecutive sections were examined on each slide, and two independent tonsils were tested.

Electrophoretic mobility shift assays (EMSAs). Nuclear extracts were prepared by hypotonic lysis, protein concentrations were quantified with Bradford reagents (BioRad, Richmond, Calif.), and EMSAs were performed in 4% nondenaturing acrylamide gels after incubation for 15 min at 37° C. with gamma-³²P-labeled probe, as previously described (Buchanan et al., 1995). The prototypic Bright binding site (a 150 base pair Bam HI-Fok I fragment called bf150) from the S107 V1 5′ flanking sequence (Webb et al., 1991) was used as a probe. In some instances, antibodies were added 5 min before incubation with the probe. Antibodies used for supershifts were: affinity purified goat anti-human Bright peptide sera, goat-anti-Bright P29 peptide sera (Webb et al., 1998), preimmune goat sera, mouse monoclonal anti-Btk (Upstate Biotechnology, Lake Placid, N.Y.), polyclonal rabbit antisera to mouse Bright (gift from P. Tucker, University of Texas, Austin, Tex.), control ascites SK7094, and polyclonal rabbit anti-CDP/Cux (gift from E. Neufeld, Yale University, New Haven Conn.) reactive with NFμNR (Wang et al., 1999). A double-stranded oligo containing the 45 base pair foot-printed Bright binding site within bf150 described in Webb et al. (1991) was used as a specific competitor in increasing concentrations and a mutated version of that oligonucleotide containing the double-stranded sequence corresponding to 5′-TAAGTATAAATATGTACATGAGTACACCCTCCACTTATTTATCTTA-3′ (SEQ ID NO:6) was used as a non-specific competitor. Underlined nucleotides represent changes from the prototype. Competition assays were performed as previously described (Webb et al., 1991).

Immunofluorescence analyses and sorting. Four-color immunofluorescence analyses were used for identification of B cell subpopulations after depletion of non-B lineage cells by magnetic separation. Viable lymphoid cells were identified using light scatter characteristics and were analyzed using a FACStar plus (Becton Dickinson) and a MoFlo (Cytomation, Fort Collins, Colo.) cell sorter with the assistance of the OMRF Flow Cytometry Core and the Flow Cytometry and Cell Sorting Laboratory, Oklahoma Center for Molecular Medicine, University of Oklahoma Health Sciences Center. Cells were stained at a concentration of 10⁷ cells/ml at 4° C. for 15 min, with the following biotinylated, FITC-, PE- or APC-conjugated antibodies: anti-CD24 (clone ML5), anti-IgM (DA4-4), anti-IgD (IA6-2), and anti-CD19 (1D3) from Pharmingen; anti-CD23 and anti-CD38 from Becton Dickinson; and anti-CD10 (5-1B4) from Caltag (Burlingame, Calif.); goat anti-human IgG and IgA from Southern Biotech; anti-CD34 (HPCA-2) from BD Biosciences (Mountain View, Calif.); and anti-CD77 from Immunotech (Westbrook, Me.). Biotinylated antibodies were revealed with Streptavidin Red 63 (Invitrogen, Carlsbad, Calif.) and isotype matched control antibodies labeled with the corresponding fluorochrome were used to determine background staining. Post-sort analyses typically yielded >95% purity in samples with cell numbers great enough to assess.

RT-PCR analyses. Total mRNA from tissues and sorted cell populations was extracted using TriReagent (MRC, Cincinnati, Ohio). Synthesis of cDNA was performed at 42° C. for 1.25 hour with avian myeloblastosis virus reverse transcriptase. Samples were amplified for 35-40 cycles of 60° C. for 30 sec, 72° C. for 2 min, and 93° C. for 30 sec. Beta-actin primers (Stratagene) were used to control for relative RNA levels and were detected by ethidium bromide staining. Bright primers used were: exons 2 through 8 forward: 5′-AGCTGCAGCCGCCTGACCAC-3′ (SEQ ID NO:7) and reverse: 5′-TGTTGGGAGCAGAGGTTGGC-3′ (SEQ ID NO:8); and exons 4 through 7 forward: 5′-GTGGCGTGAGATCACCAAG-3′ (SEQ ID NO:9) and reverse: 5′-CAGAACTCCTGTGTACATG-3′ (SEQ ID NO:10). Amplified products from selected samples were sequenced to confirm their identity. Samples were electrophoresed through 0.8% agarose gels, transferred to GeneScreen Plus hybridization membrane (NEN LifeScience Products, Boston, Mass.) for Southern blotting and the low abundance Bright transcripts were detected by hybridization with a ³²P-labeled, 1.8 kB human Bright cDNA probe.

### Example 2

### Results

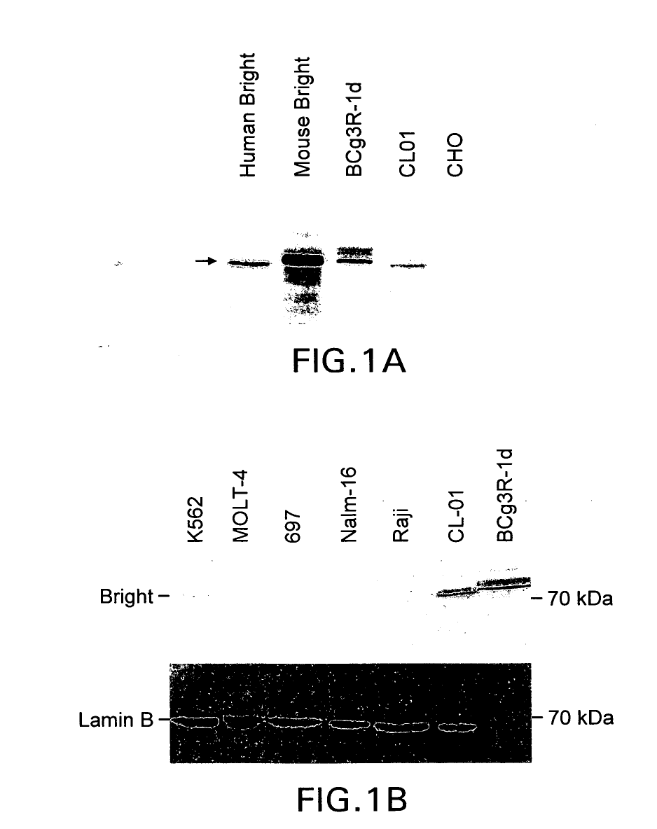

Bright protein expression in transformed cell lines. Human Bright protein is 505 amino acids long, as compared to the murine 601 amino acid form, and is 79% identical to the mouse amino acid sequence overall. Expression of recombinant mouse and human proteins exhibited one predominant band of the predicted size in a western blot (FIG. 1A). Faint higher molecular weight Bright bands were apparent in both the murine BCg3R-1d cell line and the human B cell line, CL01. The inventor previously reported the existence of two additional forms of murine Bright (Webb et al., 1998), but have been unable to demonstrate that either the human or mouse forms are the result of alternatively spliced isoforms. Treatment of the protein samples with phosphatases failed to eliminate those bands suggesting that they are not the result of post-translational phosphorylation events (Webb et al., 1998) Examination of a broad panel of B and non-B lymphocyte cell lines, represented in part by FIG. 1B, suggested that Bright protein was not expressed in many human B cell lines, including the pre-B cell line 697 and the mature B cell lines Raji, Ramos and Daudi. Likewise, Bright protein was not present in any of the T cell lines examined. However, the early eyrthroid progenitor, K562, produced Bright protein. Therefore, Bright protein expression in human B cell lines was more restricted than in the corresponding mouse cell lines.

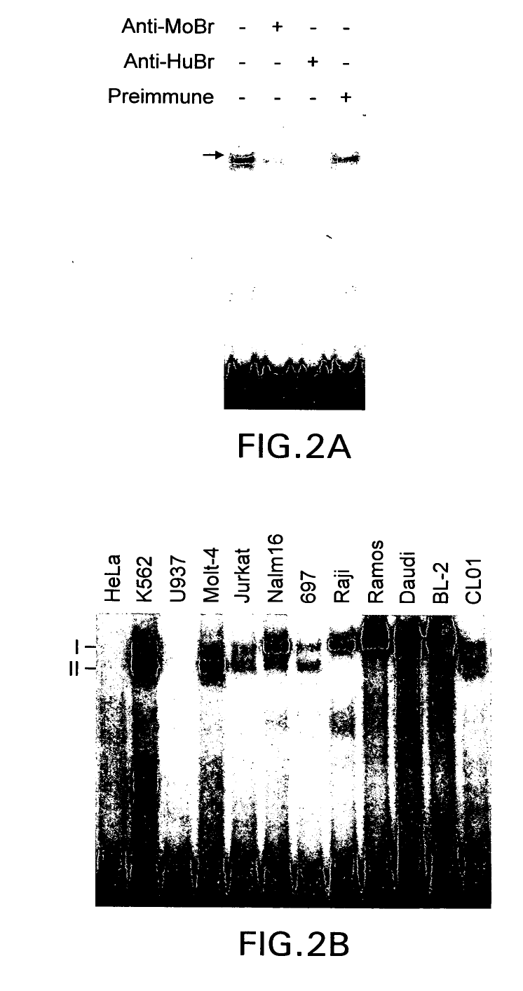

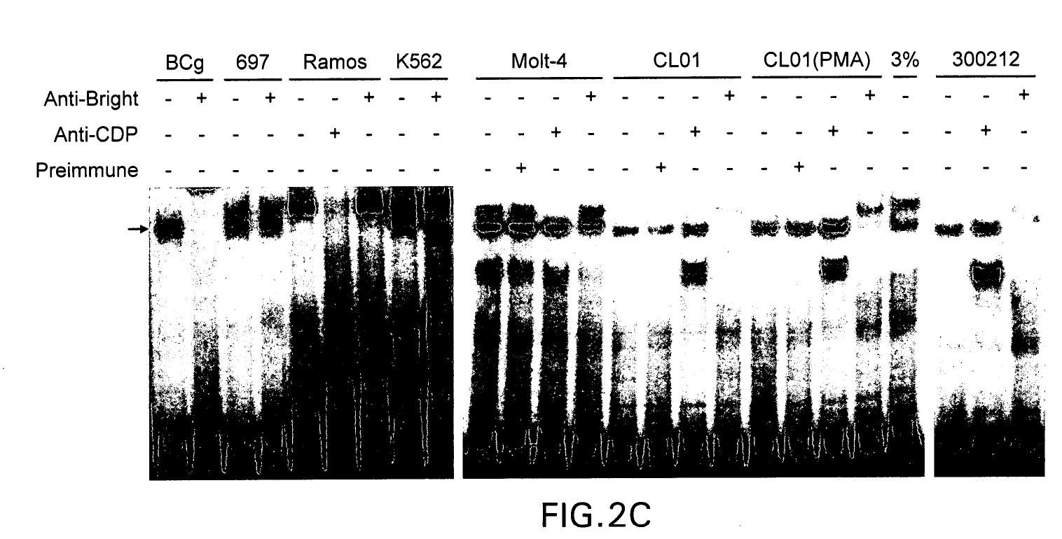

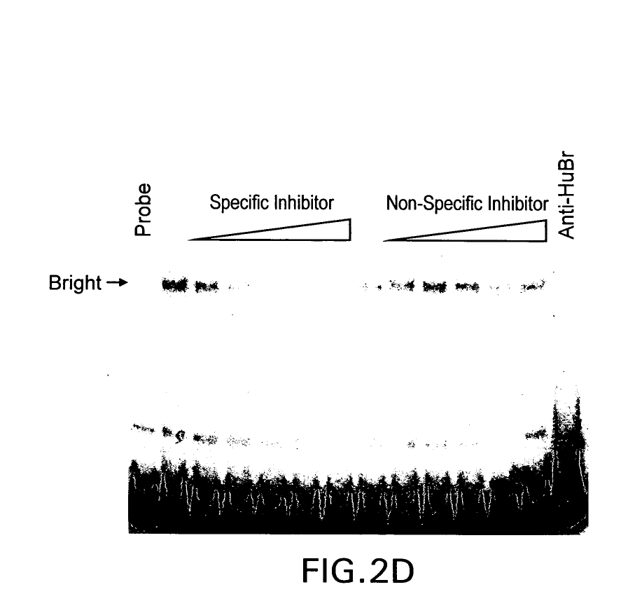

Human Bright binds the DNA consensus motif. Because the ARID DNA-binding domains of mouse and human Bright are 97% identical, the inventor predicted that human Bright should bind the bf150 Bright prototype DNA binding motif (Webb et al., 1998). Mobility shift assays demonstrated that recombinant human Bright bound the Bright sequence and reacted with both of the new anti-Bright peptide reagents the inventors produced (FIG. 2A). Preimmune serum did not supershift the Bright protein complex. Like mouse Bright (Webb et al., 1991), binding of the human protein was specifically competed with the unlabeled bf150 fragment itself, but not with an unrelated DNA fragment (not shown). Thus, as expected, mouse and human Bright exhibit similar DNA-binding activity.

Nuclear extracts from a panel of human cell lines displayed a more complicated pattern of reactivity with the Bright motif than expected. Mouse Bright DNA-binding complexes were observed in all B cell lines tested except early pre- and pro-B cell lines, but were not present in non-B lineage lines which expressed the NFμNR complex instead (Webb et al., 1998). Consistent with the inventor's findings in the murine system, fibroblasts (HeLa) and monocyte lineage cells (U937) failed to exhibit Bright binding activity (FIG. 2B). However, several of the cell lines, including CL01 and K562 which exhibited Bright protein by western blotting, formed two major complexes (labeled I and II) that reacted with the Bright sequence. Furthermore, all of the common mature B cell lines (Raji, Ramos, Daudi, BL-2), and the pro- and pre-B cell lines Nalm-6 and 697, exhibited protein complexes that bound the Bright motif (complex I, FIG. 2B), despite the absence of Bright protein in western blots (see FIG. 1B). Analyses with anti-Bright sera demonstrated that only the protein II complexes observed in the human germinal center B cell line CL01 (Cerutti et al., 1998), the multipotent progenitor line K562 (Lozzio et al., 1981) and the EBV-tranformed B cell line 300212 contained Bright protein (FIG. 2C). These findings were confirmed using antisera to three different domains of human Bright and by western blotting as shown in FIGS. 1A-B for some of the cell lines. The identity of the protein II complexes observed in the 697 and T cell lines, including Molt-4, is unknown, but the inventor speculates that they may result from other ARID family proteins in the human database.

To confirm that the protein complexes that reacted with anti-Bright antibodies in FIG. 2D were specific for the Bright binding site, competition experiments were performed in the presence of double-stranded oligonucleotides spanning the Bright binding motif. A mutated oligonucleotide of the same length was used to demonstrate the specificity of the mobility shifted complexes for the Bright binding sites. Each of the complexes that reacted with Bright sera from FIG. 2D, as well as recombinant Bright, were specifically inhibited by the Bright binding motif, but not the non-specific competitor. Data for the cell line 300212 are shown in FIG. 2D.

The slower migrating protein complex I observed in several of the extracts, including many of those from B cell lines, was reminiscent of the NFμNR repressor observed in the majority of non-B cells in the mouse (Wang et al., 1999). To determine if this complex contained CDP/Cut/Cux, like NFμNR (Wang et al., 1999), antibodies to CDP were included in the assays (FIG. 2C). Anti-CDP supershifted complex I in each of the human lines, including those observed in the B cell lines (FIG. 2C, and data not shown). Although the functional significance of this reactivity is unknown, stimulation of CL01 cells with PMA both increased Bright activity and reduced levels of the anti-CDP-reactive complex I (FIG. 2C). Likewise, CL01 cells grown in 3% serum, a condition previously shown to reduce murine Bright production, exhibited increased levels of complex I relative to Bright. Therefore, the DNA-binding activity of the CDP-like complex appears to be co-regulated with Bright, consistent with findings by us and others suggesting that Bright and NFμNR are co-regulated in the mouse (Wang et al., 1999). These data indicate that an NFμNR-like complex exists in human cells and that Bright expression in human B cell lines differs substantially from patterns observed using mouse cell lines.

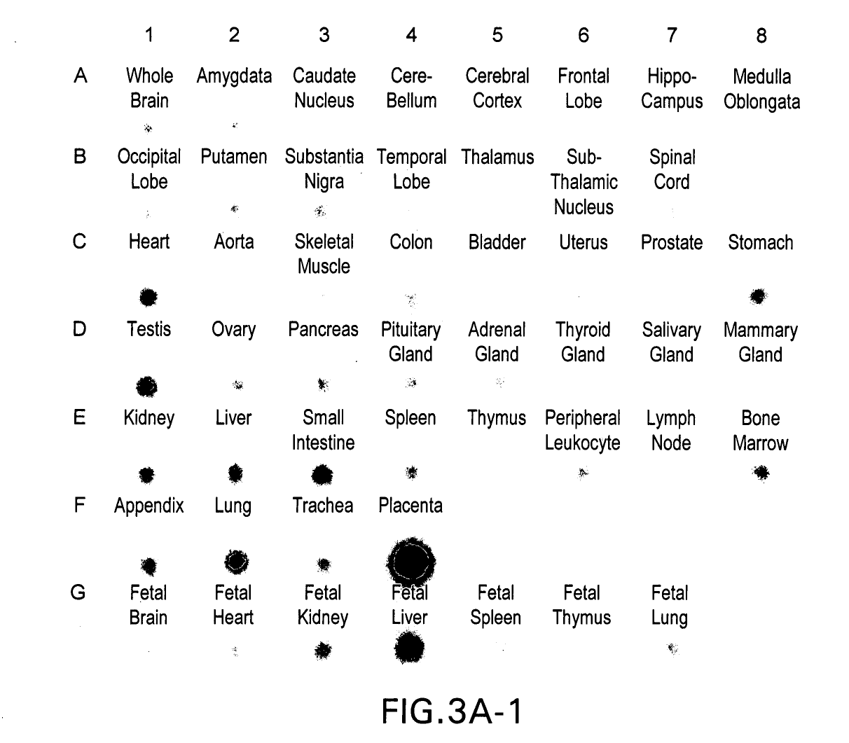

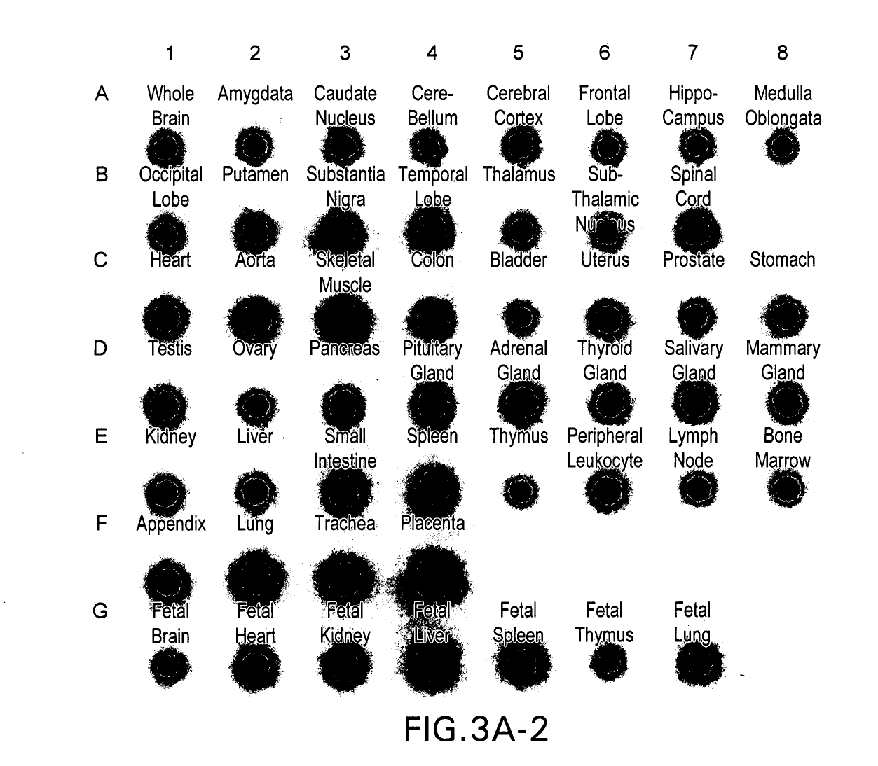

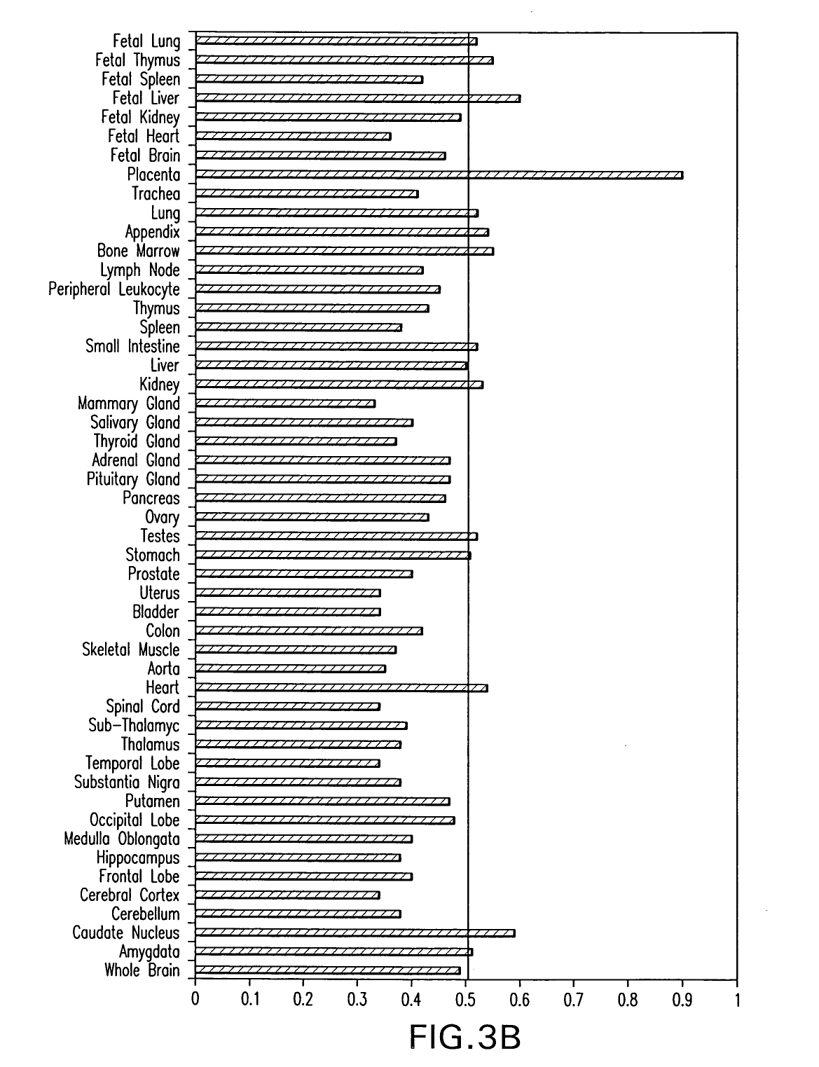

Bright expression in normal human tissues. Because the human genome contains at least 10 additional proteins with conserved ARID DNA binding domains, and the inventor's EMSA data suggested that additional ARID family members might be expressed in the cell lines examined in FIGS. 2A-C, the inventor re-examined the tissue distribution of Bright mRNA using a full-length cDNA. Previous experiments used only the ARID domain of Bright as a probe (Kortschak et al., 1998). A commercially available panel of mRNAs from human tissue was hybridized under stringent conditions with a 2 kB human Bright probe (FIGS. 3A-B). Bright expression was clearly most abundant in the placenta. However, fetal liver, bone marrow, the caudate nucleus, heart, appendix, fetal thymus, kidney, testis, small intestine, lung, fetal lung, stomach, amygdala, and liver were very weakly reactive. These data contradict earlier reports suggesting high expression in the colon (Kortschak et al., 1998), but the RNA transcript data shown in FIGS. 3A-B are consistent with the inventor's earlier observations indicating that murine Bright mRNA expression in hematopoietic tissues is not abundant (Webb et al., 1998).

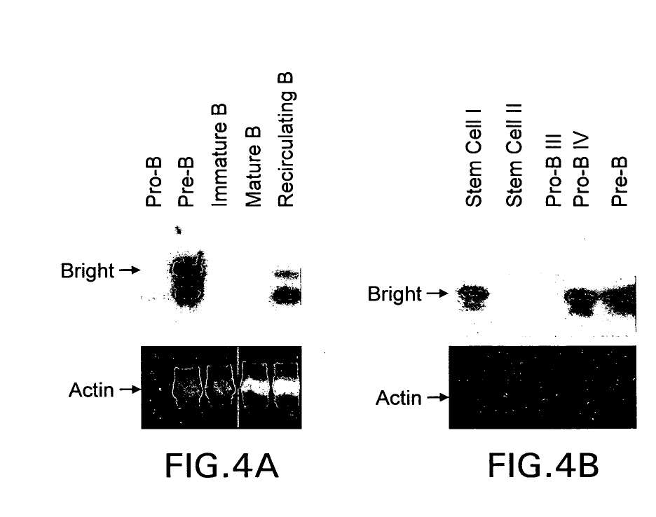

Bright is expressed in early B lymphocyte precursors. To determine whether Bright was expressed in B lineage subpopulations from the bone marrow, adult bone marrow mononuclear cells were separated into pro-B (CD34⁺, CD38⁺, CD10⁺), pre-B (CD34⁻, CD19⁺, IgM⁻, CD24⁺), immature B (CD34⁻, IgM⁺, IgD⁻, CD24^{hi}), mature B (CD34⁻, CD19⁺, IgM⁺, CD24^{hi}, IgD⁺) and recirculating B cell (CD34⁻, IgM⁺, IgD⁺, CD24^{lo}) stages, according to Rossi et al. (2001). Bright mRNA was detected by RT-PCR in pro-B and pre-B cells, as well as in recirculating B cells in the marrow, but not in immature or mature B cells (FIG. 4A). Each sample was amplified independently with both primer pairs from exons 2 and 8 and 4 and 7 to assure the presence of full length transcripts. Under the PCR conditions used, the highly related BDP cDNA clone (Numata et al., 1999) was not amplified. Further separation of early bone marrow populations into very early stem cells (stem cell I: CD34⁺, CD38⁻, CD10⁻, CD19⁻ and stem cell II: CD34⁺, CD38⁺, CD10⁻, CD19⁻ (Rossi et al., 2001) suggested that the earliest CD34⁺ stem cells produced Bright mRNA and was similar to expression in stem cells and fetal liver in the mouse (Webb et al., 1998). Division of pro-B cell populations into CD19⁻ (Pro-B III) or CD19⁺ (Pro-B IV) cells suggested that Bright expression recurred with expression of CD19 (FIG. 4B). Thus, Bright expression in the bone marrow occurs in three distinct populations in B cell differentiation, in the very early stem cell, during the pro-B to pre-B cell stage and in recirculating, antigen-stimulated cells.

Bright is expressed in germinal center cells. Further analyses were undertaken to determine whether Bright expression occurred in other lymphocyte-rich tissues. Only one of five peripheral blood samples examined contained very low levels of Bright protein by EMSA, two of three cord blood samples tested showed low levels of Bright protein by western, and none of the three thymuses examined contained detectable Bright protein. However, tonsil mononuclear cells exhibited weak protein complexes that bound to the Bright consensus site and were inhibited by interactions with anti-Bright antibodies (not shown).

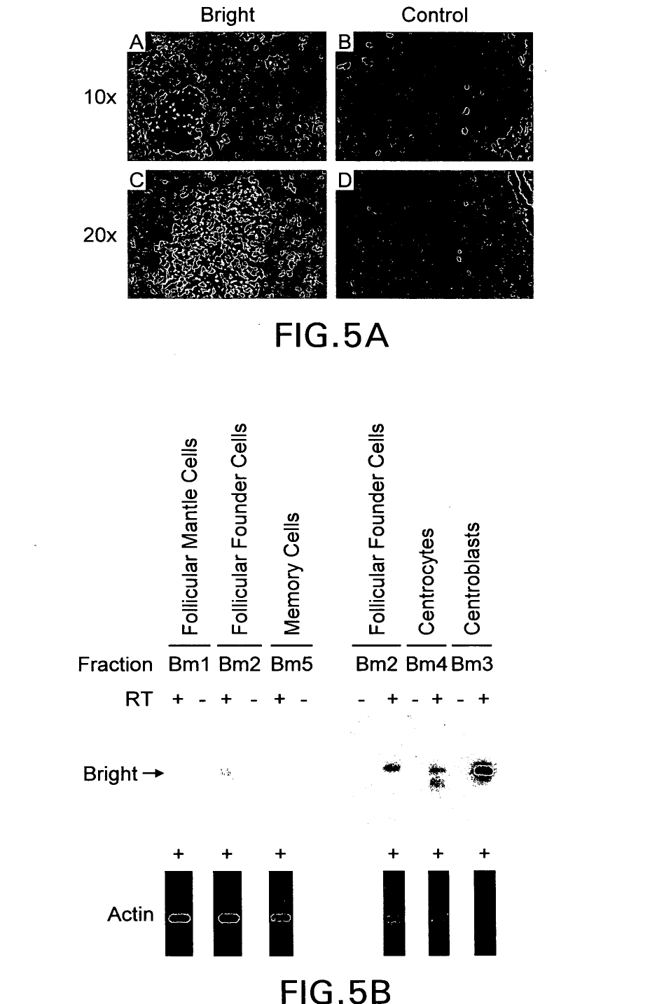

To determine if Bright expression in human tonsil was limited to germinal center cells, as the inventors had observed in the mouse spleen, tonsil sections were subjected to in situ hybridization with Bright mRNA sense and anti-sense probes. FIG. 5A shows increased hybridization (dark brown color) within the morphologically detectable germinal centers at two magnifications (left panels). The Bright sense probe did not react specifically with the germinal centers (right panels). Tonsil B lymphocytes were then fractionated into five subpopulations of B cells based on cell surface protein markers, as previously described (Pascual et al., 1994). These are: IgD⁺, CD23⁻, CD38⁻ follicular mantle cells (Bm1); IgD⁺, CD23⁺, CD38⁻ follicular founder cells (Bm2); IgD⁻, CD77⁺, CD38⁺ germinal center centroblasts (Bm3); IgD⁻, CD77⁻, CD38⁺ centrocytes (Bm4); and IgD⁻, CD38⁻ memory cells (Bm5). Although the numbers of sorted cells obtained from each population were too small to allow detection of protein by either EMSA or western, RT-PCR analyses demonstrated that Bright expression was first evident in the germinal center founder cells (Bm2), peaked in the dark zone centroblasts (Bm3), and remained at lower levels in the light zone centrocytes (Bm4) (FIG. 5B). Thus, human Bright expression is limited to subpopulations of germinal center B cells in the tonsil.

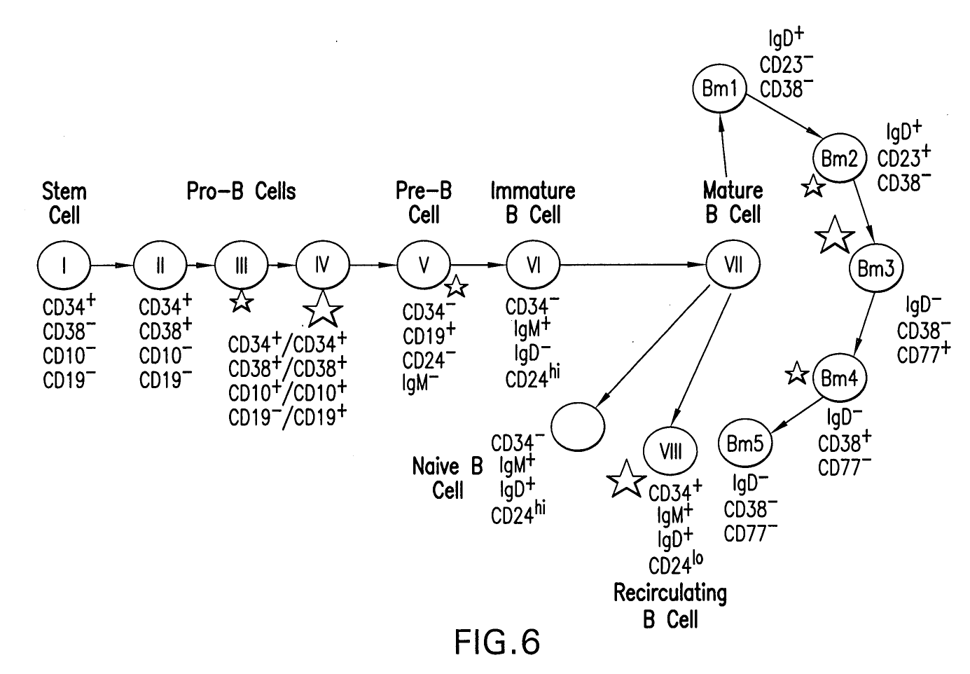

A schematic diagram of each of the human B cell subpopulations analyzed, the surface markers used to identify them, and the presence or absence of Bright mRNA is shown in FIG. 6. These data parallel earlier experiments with mouse B cells, except that human Bright mRNA expression occurred first in pro-B cells in the human rather than in the later pre-B cell stages (Webb et al., 1998).

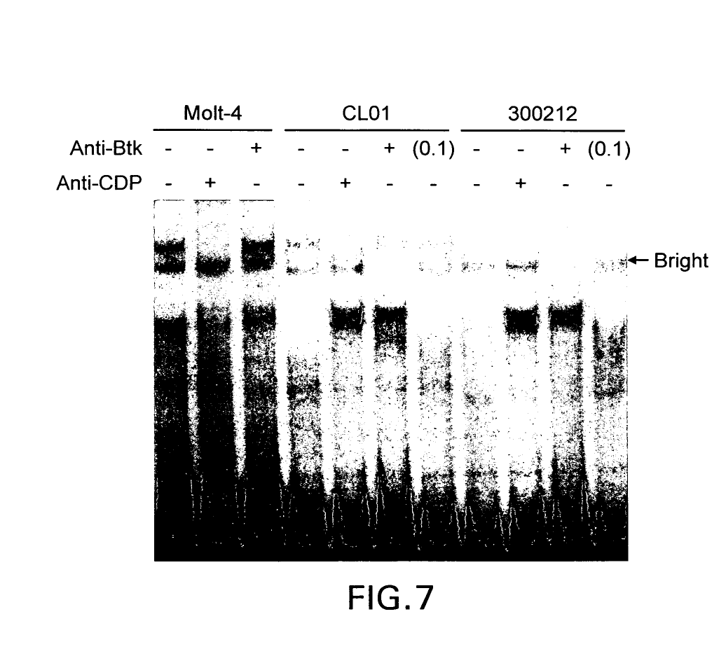

Human Bright DNA-binding complexes contain associated Btk. Bright DNA-binding complexes in the mouse contained Btk (Webb et al., 2000). Therefore, the inventor asked if Btk was also associated with human Bright. FIG. 7 demonstrates that Bright complexes reactive with anti-Btk antibodies were present in both of the B cell lines CL01 and 300212. Antibodies against the pleckstrin homology (PH) domain of Btk supershifted the Bright complex present in the CL01 cell line producing a fuzzy band that migrated just below the CDP-reactive complex. Anti-Btk also affected binding of the Bright complex from the 300212 cell line, but failed to produce a clear supershifted band at any dilution suggesting that such antibody-protein complexes may be unstable. Neither the anti-CDP sera nor an isotype-matched control ascites (not shown) affected binding of the Bright complex. In addition, none of the protein complexes observed in the Molt-4 T cell line were affected by anti-Btk indicating specific reactivity with the Bright complex. These data suggest that human Bright can exist as DNA-bound complexes associated with Btk.

### Example 3

### Discussion

The inventor's previous studies indicated a link between Bright activity in the mouse and X-linked immunodeficiency disease (Webb et al., 2000). Because humans also suffer from X-linked immunodeficiency diseases, but have a much more severe phenotype than mice, they sought to determine whether human Bright was expressed in B lymphocytes and if it associated with Btk. In addition, previous analyses of human Bright had not been extended to the protein level (Kortschak et al., 1998). The data presented here demonstrate that Bright is not expressed in all human B lymphocyte subpopulations and show that Bright/Btk associated DNA-binding complexes exist in some human B cell lines. Human and mouse Bright expression and activities in non-transformed B cells were similar, indicating that human Bright may share important functions with the mouse protein.

The human Bright protein cloned from peripheral blood lymphocytes is identical to the previously published DRIL1 sequence (Kortschak et al., 1998). Interestingly, the inventor's mRNA expression data differ from those obtained by Kortschak et al. (1998), who found that Dril 1 mRNA expression was highest in muscle, colon and thalamus. In those studies, the ARID domain was used as an mRNA probe. Differences in expression patterns using a full length cDNA probe may reflect lack of hybridization to other members of the growing number of identified ARID family proteins. For example, the BDP protein is 95% homologous to Bright within the ARID region, binds the Bright DNA sequence and is expressed ubiquitously (Numata et al., 1999). Tissue analyses suggest that human Bright may be expressed in some non-B lineage subpopulations other than stem cells. However, Bright protein expression has not been demonstrated in non-transformed B cells to date.

Human Bright is slightly smaller than the mouse protein, but binds equally well to the Bright DNA consensus motif and may exist in post-translationally modified forms, similar to those previously described in the mouse (Webb et al., 2000). The putative modified isoforms react with antibodies prepared against three different polypeptides of Bright, but are not affected by treatment with phosphatases (Webb et al., 2000). Therefore, the larger isoforms were not the result of tyrosine phosphorylation, but the type of modification resulting in a larger apparent size remains unclear.

Bright expression in non-transformed human cells was similar to that observed for mouse Bright. Both mouse and human Bright mRNA were expressed in pre-committed lymphocyte progenitors, in pre-B cells, and in germinal center cells. The results from those experiments are diagrammed in FIG. 6. Expression of human Bright mRNA in germinal centers peaked during the centroblast and centrocyte stages. These data contrast with microarray analyses from two other labs where Dril 1 hybridization above baseline was not detected in either centroblast or centrocyte stages, although the inventor's data for memory B cells is consistent with the observations of Alizadeh et al. (2000); Klein et al. (2003). The reasons for the discrepancy in the germinal center subpopulations are unclear, but may result from differences in the cell populations assessed, or from the fact that the microarray data was obtained from hybridization with relatively small oligonucleotides from the human Bright sequence.

The inventor's data was obtained using oligonucleotides that spanned all eight exons and reflects the presence of full-length mRNA for Bright. In the inventor's hands, use of oligonucleotides within the ARID domain or the highly conserved protein-interaction domains often resulted in expression patterns that did not correlate with results found with oligonucleotides spanning the entire coding sequence. The RT-PCR data are consistent with previous observations in the mouse, where the peanut agglutinin-high germinal center cells were shown to express abundant Bright activity, while Bright was not present in most of the splenic B cells (Webb et al., 1998). Neither immature nor mature peripheral blood B cells in the human expressed detectable mRNA for Bright. While splenic B cells in the mouse can be induced to express Bright with a number of stimuli, including LPS, CD40 ligand and interleukin-5 plus antigen (Webb et al., 1998), Bright expression in human peripheral blood cells was not induced by any of the common mitogens including PMA, LPS and pokeweed mitogen (not shown). However, Bright expression was increased in the CL01 cell line after treatment with PMA and induction did not require the calcium ionophore, ionomycin (FIG. 2C). These data suggest that Bright expression during human B lymphocyte development is tightly regulated at the level of transcription, as it is in the mouse.

By far, the biggest difference observed between mouse and human Bright expression occurred in transformed cell lines. Contrary to mouse B cell lines that uniformly express Bright, many human B cell lines did not express Bright protein. In addition, the pro- and pre-B cell lines Nalm-16 and 697 did not express Bright protein (FIGS. 1A-B and unpublished results), although non-transformed pro- and pre-B cells produced abundant mRNA for Bright. However, all of the lymphocytic lines expressed proteins that bound the Bright DNA-binding motif. Several of the B and non-B cell lines expressed proteins that migrated similarly to Bright in EMSAs, but did not react with anti-Bright antibodies. These proteins may represent other members of the rapidly growing ARID family, but they failed to react with anti-BDP serum (data not shown).

Furthermore, most of the cell lines expressed varying levels of a CDP-related protein complex reminiscent of the murine NFμNR repressor complex that competes for Bright binding sites in the mouse immunoglobulin locus and inhibits Bright-induced transcription (Wang et al., 1999). It is not clear why NFμNR-like complexes should be expressed in human B cell lines. The human B cell lines express surface immunoglobulin so this CDP-related complex does not appear to repress the immunoglobulin locus in these cells. Nonetheless, the inventors have observed an inverse relationship between Bright and this complex in the CL01 cell line (FIG. 2C), suggesting that these complexes may be co-regulated. Alternatively, the NFμNR-like complexes may be functionally distinct from those observed in the mouse.

Mobility shift assays demonstrated human protein complexes similar to those observed with mouse Bright that reacted with antibodies to both Bright and Btk (Webb et al., 2000). The function of Bright in the mouse is to increase immunoglobulin heavy chain transcription (Herrscher et al., 1995; Webb et al., 1991), and Bright should play a similar function in human cells. Others have shown that T cell receptor levels must be maintained above a specific threshold for T cell maturation to progress properly. In keeping with these data, Tec kinases were important for upregulation of TCR transcription (Novina et al., 1999; Cheriyath et al., 1998). Therefore, Bright, and the associated Tec kinase member, Btk, may be necessary at the pre-B cell and germinal center stages to ensure that surface immunoglobulin levels in actively dividing and differentiating cells are maintained above threshold values.

Earlier studies in the mouse suggested a link between Bright and the xid defect (Webb et al., 2000). The association between human Bright and Btk suggests another link between Bright function and X-linked immunodeficiency disease. Also consistent with the idea that Bright may be linked to the human disease, XLA, human Bright expression first occurred in pro-B cells. This is slightly prior to the early large pre-B cell stage where Bright was first expressed in the mouse (Webb et al., 1998). The block in B cell differentiation in XLA occurs at the pro-B to pre-B cell transition, while in xid mice, B cells accumulate at the immature stage (Conley et al., 1994; Satterthwaite and Witte, 1996). However, recent studies suggest that xid B cells also exhibit subtle defects at the pro-B to pre-B cell transition (Kouro et al., 2001; Middendorp et al., 2002).

### Example 4

### Materials and Methods

Cell lines and transfections. Chinese hamster ovary cells (CHO) were maintained in DMEM supplemented with 7% heat inactivated fetal calf serum (FCS), 100 U/ml penicillin, 100 μg/ml streptomycin, 5×10⁻⁵ M 2-ME, and 1 mM sodium pyruvate. The M12g3Ri and BCg3R-1d cell lines, transfected derivatives of the B cell lines M12.4 and BCL1B1, respectively, contained coding sequences for a T15 idiotype immunoglobulin (Webb et al., 1989), and were maintained in RPMI 1640 with the same supplements. CHO cells were transfected using Fugene (Boehringer Mannheim, Indianapolis, Ind.) according to the manufacturer's directions. M12g3Ri cells were transfected by electroporation at 0.24 kV with a Gene Pulser (BioRad, Richmond, Calif.). Transfected cells were maintained in complete RPMI 1640 with 10% FCS. In some cases, cells were stimulated for 48 hours with 10 mg/ml LPS (Sigma, St. Louis, Mo.) to induce endogenous Bright expression after transfection. Transfected cells were enriched by sorting for GFP expression using an advanced MoFlo cell sorter (Cytomation, Inc., Fort Collins, Colo.) at the Flow Cytometry, Cell Sorting, and Confocal Microscopy Laboratory, Oklahoma Center for Molecular Medicine, University of Oklahoma Health Sciences Center and the Oklahoma Medical Research Foundation Flow Cytometry Core facilities. Typical sorting experiments yielded cells >90% enriched for GFP expression.

Column chromatography. Nuclear extracts from CHO cells transfected with the full-length Bright expression plasmid (Herrscher et al., 1995) were centrifuged at 10,000×g to remove aggregates and applied to a pre-calibrated Bio-Gel A 0.5 m size exclusion column (Bio-Rad, Hercules, Calif.) in 0.05 M Tris-HCl, pH 8.0, 0.1 M KCl and 20% glycerol according to the manufacturer's directions. The column was calibrated before and after use with Bio-Rad gel filtration standards to ensure maintenance of column integrity. 100 μl fractions were collected, and subjected to western blotting for Bright as previously described (Webb et al., 2000).

Mutant construction and expression vectors. A full-length mouse Bright cDNA clone in the pBKCMV (Herrscher et al., 1995) was used as a template to introduce single amino acid changes or small deletions in the Bright coding sequence through site-directed mutagenesis with the QuickChange Site Directed mutagenesis Kit from Stratagene (La Jolla, Calif.). The PCR conditions used were: 95° C. for 40 sec followed by 17 cycles of 95° C. for 40 sec, 60° C. for 1 min, and 68° C. for 16 min with an additional 20 min at 68° C. to complete the reaction. Mutations produced were: GCG to GCC, at amino acid position 286 (P286A); TGG to GCG, W299A; TTC to GCA, F317A; TAT to GCA, Y330A; REKLES at amino acids 455-460 was changed to AEALEA; and the nucleotides encoding the amino acids IKK at positions 402-404 were deleted. A double mutant (DP) was also generated that contained both the W299A and Y330A mutations. All sequences were verified using the OMRF sequencing facility and Vector NTI software.

Wild-type and mutant Bright were tagged on the carboxyl terminus with the myc-his sequence using the vector pcDNA4/TO/myc-His B (Invitrogen, Carlsbad, Calif.). Briefly, Bright was PCR amplified with oligonucleotides that added a 5′ EcoR I site and a 3′ Xba I site while deleting the endogenous stop codon, and was ligated into the EcoR I and Xba I digested vector fragment. Sequences were further subcloned into the MiGR1 retroviral vector allowing green fluorescent protein (GFP) expression from an internal ribosomal entry site to allow visualization of cells expressing Bright (Pear et al., 1998). A μ heavy chain expression vector containing the V1 S107 family sequence from −574 to +146 of the coding sequence and including the previously described Bright binding sites and a 1 kb Xba I fragment including the complete intronic heavy chain enhancer sequence (Webb et al., 1991) was used as a reporter construct. Other plasmids used were GFP-Btk (Webb et al., 2000), a MiGR1 vector containing the Bright sequence in the reverse orientation and pUC19.

Electrophoretic Mobility Shift Assays (EMSAs). Nuclear extracts were prepared by hypotonic lysis with the protease inhibitors PMSF (5×10⁻⁵ M), leupeptin (1×10⁻² mg/ml) and aprotinin (5×10⁻³ mg/ml), as previously described (Buchanan et al., 1995) and protein concentrations were determined using Bradford reagent (BioRad). Proteins were incubated with a γ-³²P-labeled DNA probe at 37° C. for 15 min and EMSAs were performed in 4% nondenaturing acrylamide gels, as previously described (Webb et al., 1991). The DNA probe was a 150 bp BamH I-Fok I fragment from the S107 V1 5′-flanking sequence (bf150) (Webb et al., 1991) containing the prototypic Bright binding site. For supershift assays, antibody and proteins were pre-incubated 5 min prior to probe addition. Antibodies used were polyclonal rabbit anti-mouse Bright (gift of P. Tucker, University of Texas, Austin, Tex.), mouse monoclonal IgG₁ anti-myc (Invitrogen), polyclonal rabbit anti-CDP/Cux (gift of E. Neufeld, Yale University, New Haven, Conn.), and preimmune serum.

Confocal microscopy and flow cytometry. Cells were harvested, washed in PBS with 3% FCS and sorted for GFP expression as previously described (Webb et al., 2000). After fixation in 1% paraformaldehyde for 15 minutes at 37° C. cells were stained with polyclonal affinity purified goat anti-peptide mouse Bright against the peptide ALHGSVLEGAGHAE (SEQ ID NO:4) from the amino terminal domain of Bright. Preimmune sera were collected prior to immunization and IgG was isolated as a negative control. Incubation with primary antibody was followed by rabbit anti-goat IgG-Alexa568 (Molecular Probes, Eugene, Oreg.) for 15 min each on ice. DAPI (Molecular Probes) was added to the cells for 2 min prior to the final washes to distinguish nuclear from cytoplasm staining. Zeiss LSM510 confocal microscope was used for analysis. Sections of approximately 50 μm were taken and analyzed using Zeiss LSM Image Browser software (Carl Zeiss, Inc., Thornwood, N.Y.) with the aid of the Oklahoma Medical Research Foundation Imaging Core Facility. Phycoerythrin conjugated streptavidin (Caltag) anti-CD19 biotin, anti-CD138 (B-D Pharmingen), anti-IgD and anti-IgM (Southern Biotech), were used for flow cytometric analyses.

Western blotting and immunoprecipitation. In vitro translated proteins were produced with TNT rabbit reticulocyte lysates (Promega, Madison, Wis.). Protein samples were subjected to SDS polyacrylamide gel electrophoresis under standard denaturing conditions through a 7.5% acrylamide gel and transferred to nitrocellulose membranes as previously described (Webb et al., 2000). Bright was detected with rabbit anti-Bright or anti-myc (Invitrogen) followed by alkaline-phosphatase conjugated goat anti-rabbit IgG (Southern Biotechnology, Birmingham, Ala.) or rabbit-anti-mouse IgG1 (Invitrogen). Blots were developed with alkaline phosphatase substrate (BioRad). Preimmune goat sera or mouse anti-Sp1 (Santa Cruz Biotechnology, Santa Cruz, Calif.) were used as isotype matched controls.

Proteins were immunoprecipitated with Protein A/G Plus-agarose beads (Santa Cruz Biotechnology) after incubation with antibody in phosphate-buffered saline (PBS) containing 0.1% Tween 20 for 1.25 hrs at room temperature with slow rotation. Bead-complexes were washed extensively with 0.1M Tris, 0.5M NaCl, and 0.1% Tween 20 before suspension in SDS sample buffer. Samples were heated for 5 minutes at 95° C., centrifuged briefly, and the supernatants were analyzed by western blotting.

Retroviral vectors and transductions. EcoPack2 cells were maintained in DMEM supplemented with 10% FCS, 100 U/ml penicillin, 100 μg/ml streptomycin, 1 mM sodium pyruvate, and 2 mM L-glutamine and were transfected with MIgR1 constructs using standard calcium phosphate procedures (Pear et al., 1993). Supernatants were harvested and virus titers were determined by infection of 3T3 cells using the method (Pear et al., 1993). Titers of 1-5×10⁶ were obtained routinely. Splenic B lymphocytes from 6-10 week old C57/B6 mice were isolated as previously described (Webb et al., 2000) and stimulated for 24 hrs prior to transduction with 25 μg/ml LPS. Transductions were performed with 8 μg/ml polybrene according to Krebs et al. (1999). Briefly, cells were resuspended at 1×10⁶ cells/ml and centrifuged with 2 ml of viral supernatant for 30 min at 300×g at 32° C., followed by a 30 min incubation at 37° C., and centrifugation with an additional 2 ml of viral supernatant. Cells were incubated at 37° C. for 8 hrs, were washed to remove the polybrene and were resuspended in RPMI-1640 (20% FCS) for 48-72 hrs. Cells were harvested and GFP expressing cells were isolated by flow cytometry.

Rear Time and RT-PCR. RNA was isolated using TriReagent according to manufacturer's protocol (MRC, Cincinatti, Ohio), treated with Dnase (2 U) (Ambion, Austin, Tex.) for 30 min at 37° C., subjected to phenol-chloroform extraction and used to generate cDNA with the SuperScript II Rnase H Reverse Transcriptase kit (Invitrogen). Immunoglobulin transcription was measured by Real Time quantitative RT-PCR using TaqMan Universal PCR Master Mix (Applied Biosystems) with 250 nm of specific primers and 5 pmoles of TaqMan probe designed using the PrimerExpress software (Applied Biosystems) and obtained from Applied Biosystems or IDT DNA Technologies (Washington D.C.).

The primers and probes used for GAPDH were: The primers for the mouse μ heavy chain allowed amplification across the intron between exons 1 and 2 and were: forward—5′CAAAATCCACTACGGAGGCAA3′ (SEQ ID NO:11) and reverse—5′TCCCGTGGTGGGACGA3′ (SEQ ID NO:12). The specific probe used for mouse μ heavy chain was 5′ATGTGCCCATTCCAGCTGTCGC3′ (SEQ ID NO:13). Expression of the V1 reporter construct was measured using the following primers and probe: forward—5′TGTCCTGAGTTCCCCAATGG3′ (SEQ ID NO:14); reverse—5′AAACCCAGTTTAACCACATCTTCAT3′ (SEQ ID NO:15); and probe—5′CACATTCAGAAATCAGCACTCAGTCCTTGTCA3′ (SEQ ID NO:16). Amplified products were examined by ethidium gel electrophoresis and the sequences were confirmed by DNA sequencing by the Oklahoma Medical Research Foundation sequencing core. Amplification reactions (25 ml) were performed in triplicate in 96-well plates under the following conditions: 50° C. for 2 min, 95° C. for 10 min, and 40 cycles of 95° C. for 15 sec and 55° C. for 1 min. and analyzed with the ABI Prism 7700 SDS (PE Applied Biosystems, Foster City, Calif.). Calculations were performed according to the equation: 2^{-ΔΔCT} as suggested by the manufacturer. For example, ΔC_{T} is the change in cycle threshold between μ and GAPDH expression; ΔΔC_{T}=ΔC_{T,q}−ΔC_{T,cb} where ΔC_{T,q} is the value obtained for the DP vector and ΔC_{T,cb} is the value for the empty vector control. In addition, the expression of J chain, Pax-5 and actin were also assessed by conventional PCR using serially diluted cDNA. PCR primers for J chain are described by Lee et al. (2003); Pax-5 primers actin primers were described (Lin and Grosschedl, 1995; Webb et al., 1998). PCR was performed with the reaction conditions at 93° C. for 1 min followed by 35 cycles (Pax-5 and actin) or 40 cycles (J chain) of 93° C. for 30 sec, 55° C. for 30 sec, and 72° C. for 30 sec with a final extension period at 72° C. for 2 min.

### Example 5

### Results

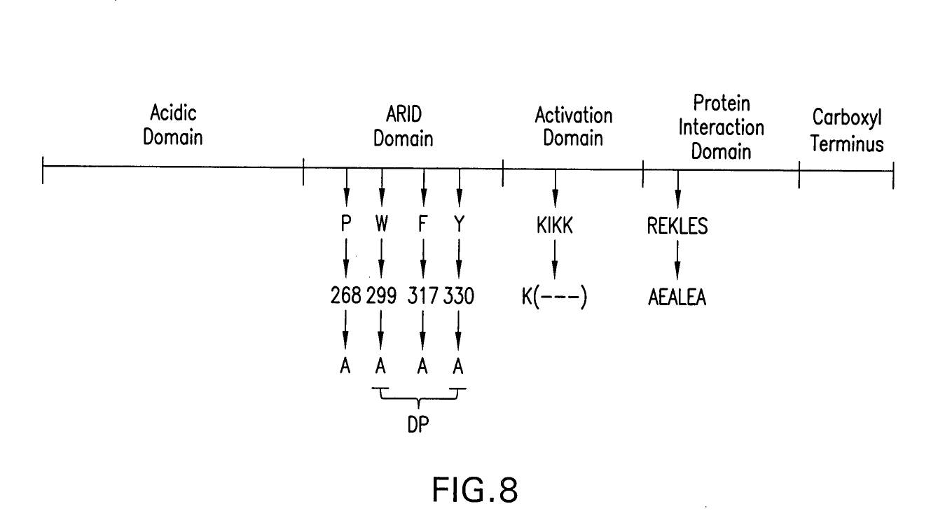

Creation of Bright mutants. Bright has been subdivided into five protein domains according to predictions from amino acid homology and analysis of deletion mutants (Herrscher et al., 1995). The domains are depicted in FIG. 8 and include an acidic amino terminus of unknown function, the ARID domain predicted to be important for DNA binding activity, a putative activation domain containing a consensus nuclear localization sequence, a protein/protein interaction domain with a helix-turn-helix structure (Suzuki et al., 1998), and a short carboxyl terminus. Site-directed mutagenesis was used to create amino acid changes hypothesized to affect the DNA-binding function of Bright. Specifically, four charged residues within the ARID domain predicted to be involved in DNA interactions (Iwahara and Clubb, 1999) were independently mutated to alanine (FIG. 8), or were introduced simultaneously into a fifth mutant to create a double mutant (DP). In addition, the putative nuclear localization sequence KIKK was altered by deleting the last three amino acids of that sequence to produce proteins predicted to sequester endogenous Bright in the cytoplasm (FIG. 8). Finally, because several ARID family members, including Bright, exhibit extended regions of amino acid homology within the putative protein interaction domain (Herrscher et al., 1995; Iwahara and Clubb, 1999), mutations were also made in the protein interaction domain to change the charged amino acids in the sequence REKLES to the sequence AEALEA.

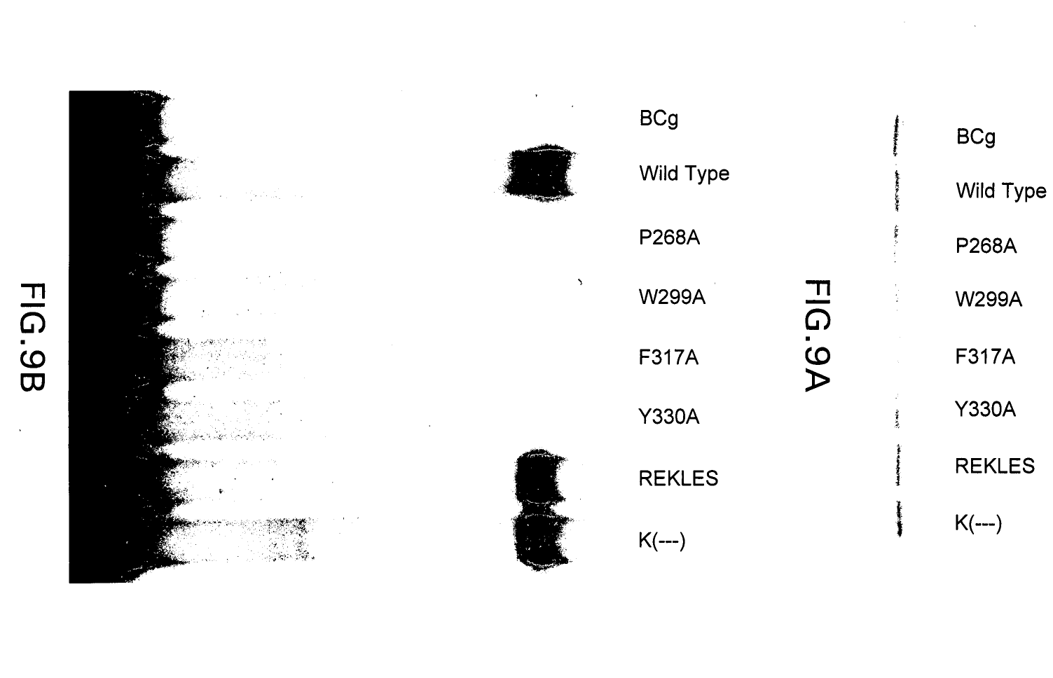

Mutations in the ARID domain decrease DNA-binding activity. All Bright mutants were expressed in an in vitro transcription and translation system and assessed by western blotting for equivalent levels of protein expression (FIG. 9A). Further analyses of these in vitro translated mutant proteins by EMSA indicated that each of the mutants within the ARID domain failed to bind DNA (FIG. 9B). However, as predicted, mutation of the putative nuclear localization sequence (KIKK) did not affect the DNA binding activity of the protein in vitro. Furthermore, alteration of the REKLES sequence was insufficient to prevent DNA-binding (FIG. 9B).

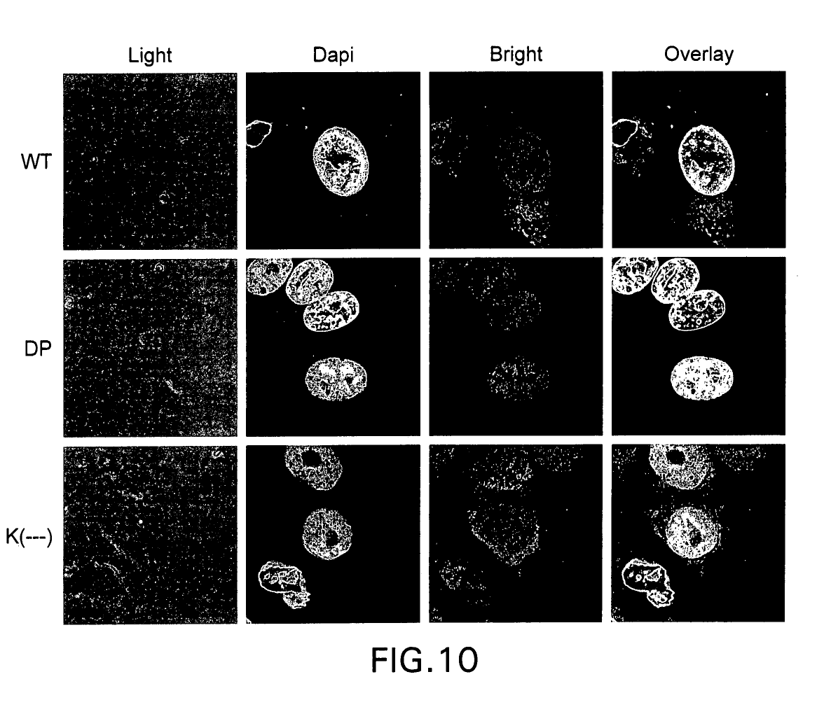

Intracellular localization of Bright. Endogenous Bright exists in both the cytoplasm and nucleus in B lymphocytes where it associates with nuclear matrix proteins (Kaplan et al., 2001). To determine if mutant Bright proteins exhibited altered intracellular localization, CHO cells were transfected with a construct that produced both GFP and histidine-myc-tagged Bright proteins. Transfected cells were identified by GFP expression and Bright was illuminated using an anti-Bright antibody and a secondary antibody conjugated to Alexa-568 (FIG. 10A). Confocal microscopy comparisons of cells transfected with wild-type Bright or the ARID mutations showed similar staining patterns with Bright expression largely in the nucleus. Therefore, mutation of the ARID domain did not adversely affect the ability of Bright to translocate to the nucleus. Staining of cells expressing the K(---) mutant showed higher levels of cytoplasmic staining than was observed with either the wild-type or ARID mutants; however, there was also some Bright staining within nuclear regions. Thus, sequences other than the KIKK consensus may also contribute to the nuclear localization of Bright.

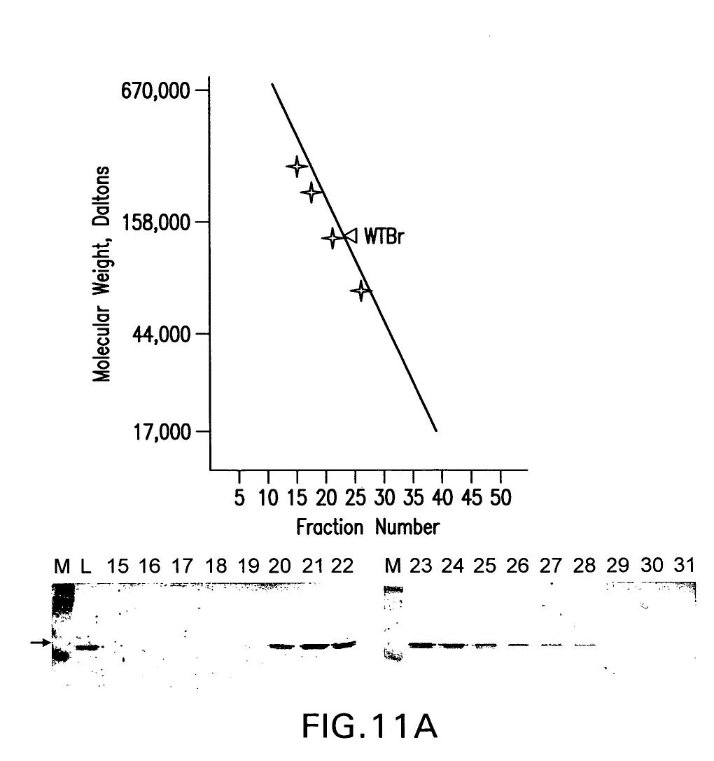

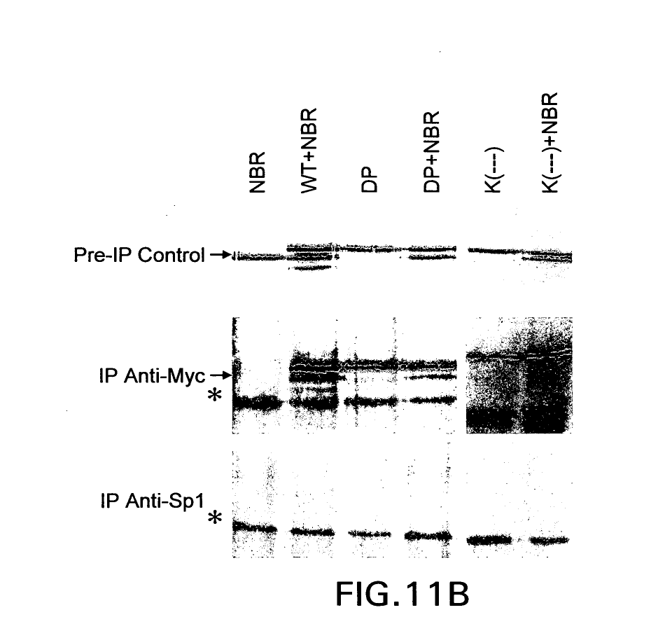

Bright exists as a dimer and can form heteromeric complexes with mutant proteins. Previous data showed that Bright cannot bind DNA as a monomer and suggested that it bound DNA as a tetramer (Herrscher et al., 1995). However, size exclusion chromatography of nuclear extracts from both transfected CHO cells and the B cell line BCg3R-1d suggested that Bright existed in solution as a dimer (FIG. 11A). No Bright protein was evident in fractions corresponding to molecular weights consistent with tetramer formation, although some Bright protein may exist in a monomeric form. Alteration of the REKLES sequence did not interfere with dimer formation (not shown).

To determine if the mutated proteins formed heterodimers with wild-type Bright, his-myc-tagged mutant forms of Bright were co-expressed in CHO cells with native wild-type Bright. Tagged proteins were distinguished from native Bright by the slight size differences in western blots (FIG. 11B) and were confirmed using anti-tag antibodies (not shown). Anti-myc antibodies were used to immunoprecipitate the tagged proteins and blots were developed with anti-Bright (FIG. 11B). In every case, a lower molecular weight band was detected that reflected the presence of native Bright. The presence of the his-myc tag did not interfere with dimer formation. This suggests that each of the mutant tagged proteins successfully formed heteromeric complexes with native Bright. These data confirm the existence of Bright dimers.

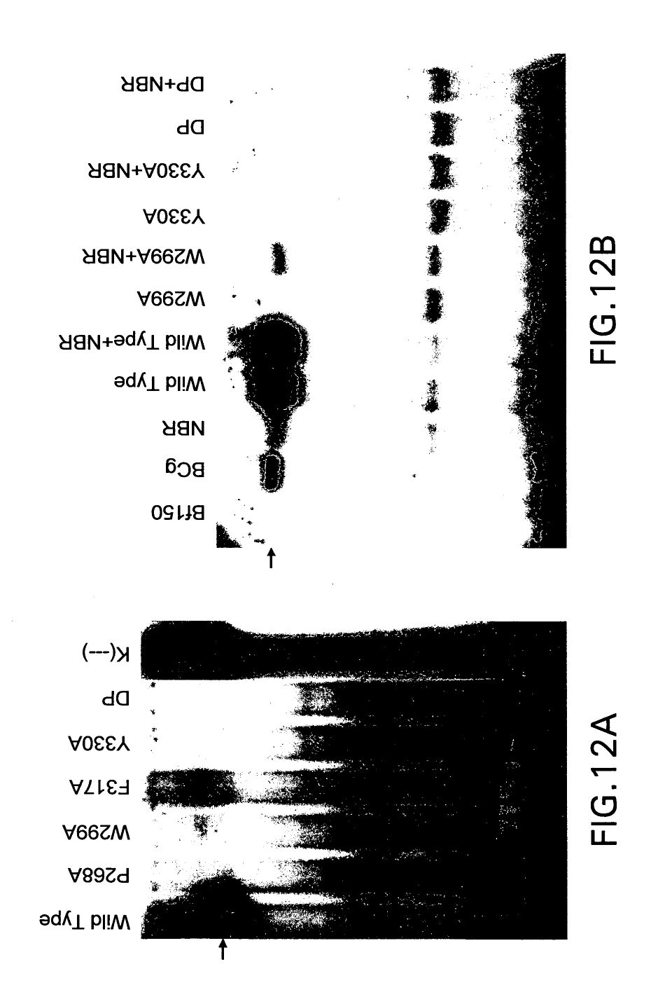

ARID/wild-type heterodimers show altered DNA-binding activity. Proteins from whole cell extracts of CHO cells cotransfected with wild-type and mutant Bright were analyzed by EMSA to determine if overexpression of mutant Bright affected wild-type Bright activity. Extracts from CHO cells transfected with only the ARID C-terminal myc-his tagged mutants exhibited similar DNA binding profiles as previously shown for the corresponding in vitro translated proteins (FIG. 12A). While the W299A mutant alone did not bind DNA (FIG. 12B) it did not efficiently inhibit the DNA binding activity of the wild-type protein. On the other hand, the Y330A mutant abolished wild-type binding completely and DP mutant inhibited the majority of Bright DNA binding activity. Therefore, some of the ARID mutants function as dominant-negative proteins.

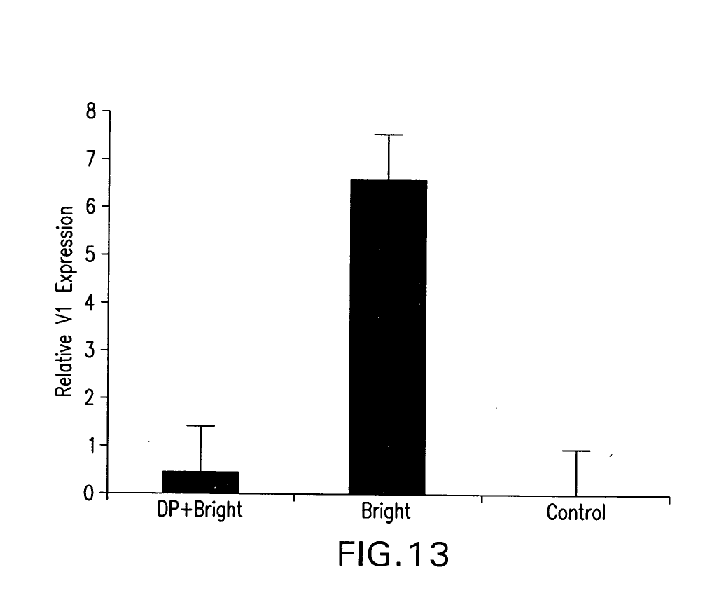

Dominant-negative Bright mutants fail to activate immunoglobulin transcription. Wild-type Bright and the dominant negative DP mutant were transiently expressed in CHO cells that do not express either Bright or immunoglobulin along with a V1 heavy chain reporter plasmid regulated by Bright (Webb et al., 1991). Real Time PCR analyses of V1 immunoglobulin transcripts indicated that wild-type Bright increased transcription of the V1 heavy chain immunoglobulin locus by 5- to 7-fold (FIG. 13). This level of transcription induction is consistent with previous observations of Bright activity (Herrscher et al., 1995; Lorch et al., 1987). However, when the DP mutant was co-expressed with wild-type Bright, no increase in transcription was observed. Therefore, the dominant negative Bright protein interfered with the transcription activation function of Bright, presumably by producing sterile Bright heterodimers that did not bind DNA (FIGS. 12A-B).

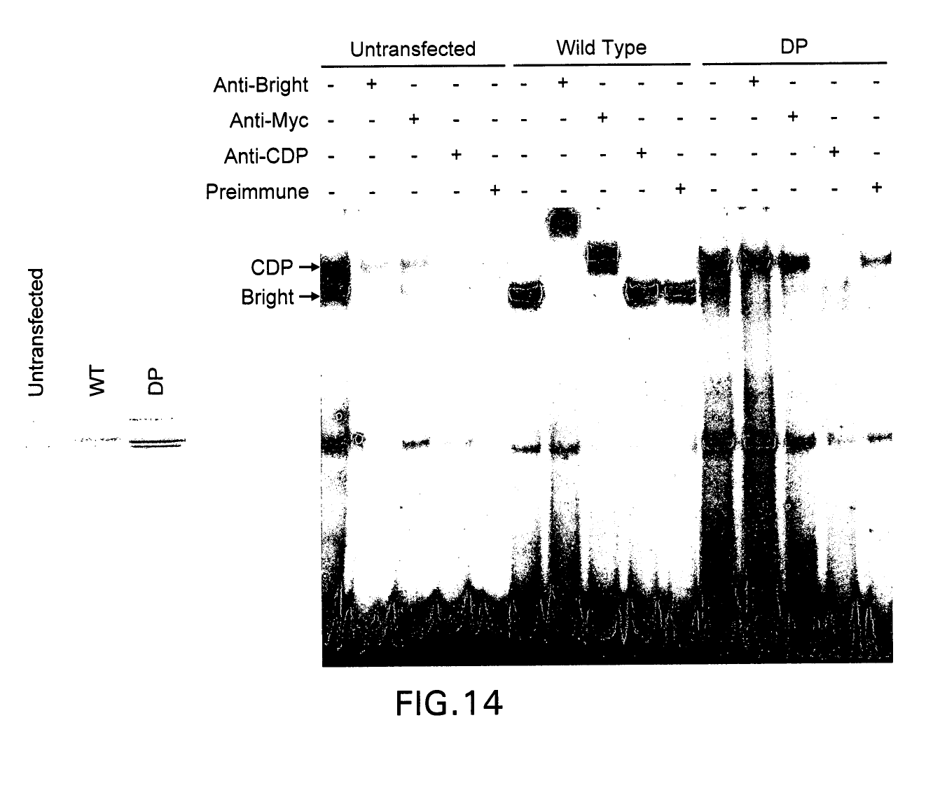

Mutants inhibit endogenous Bright DNA binding activity in B cells. To determine if these mutants also functioned as dominant negatives by inhibiting endogenous Bright function in B lymphocytes, the myc-his tagged proteins were expressed in M12g3Ri cells that constitutively express low levels of endogenous Bright. Cells were stimulated with LPS after transfection to induce new production of native Bright protein and were isolated by flow cytometry based on GFP expression. Nuclear proteins were isolated from the sorted cells, assessed by western blot and analyzed by EMSA for Bright DNA-binding activity (FIGS. 14A-C).

As demonstrated by western blot, equivalent levels of Bright are expressed in all three samples, and the levels of protein from transfected Bright are also similar (FIG. 14A). Untransfected control cells exhibited endogenous Bright activity, as did extracts from cells transfected with the tagged-WT Bright vector (FIG. 14B). Anti-Bright supershifted the Bright band in both cases. Addition of anti-myc antibody to the WT protein also supershifted the Bright band and decreased the level of endogenous Bright binding activity. This suggests that the tagged Bright interacted with endogenous Bright in the B cell.

Consistent with those findings, extracts from cells expressing DP Bright exhibited very low levels of Bright DNA-binding activity that did not react with anti-myc antibodies and likely reflects endogenous Bright dimers formed prior to LPS stimulation. In each case, the upper mobility-shifted complex reacted with antibodies to CAAT displacement protein (CDP). This protein complex was previously described by this lab and others as a protein complex that competes for Bright activity and binds to DNA sequences that overlap Bright (Herrscher et al., 1995; Wang et al., 1999; Webb et al., 1991). Nevertheless, these experiments suggest that DP Bright functions as a dominant negative in B cells by interfering with newly expressed endogenous Bright DNA-binding activity.

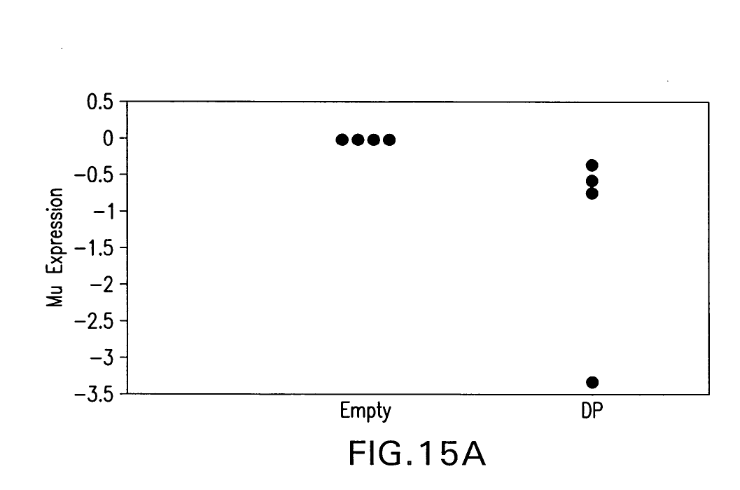

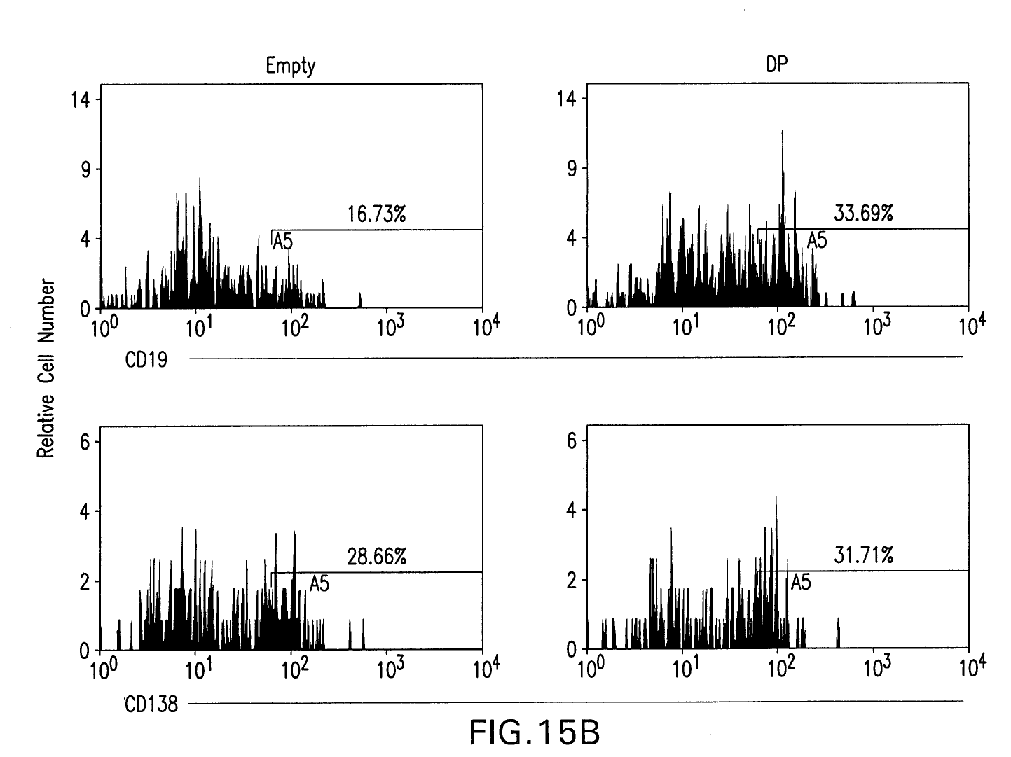

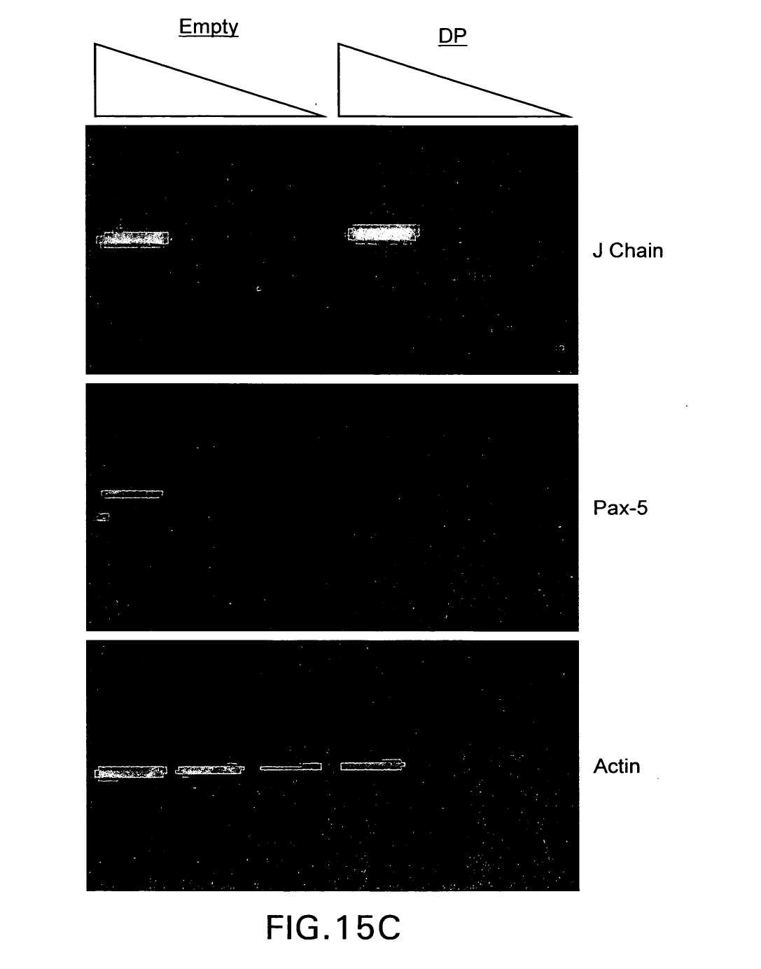

Inhibition of Bright activity in LPS-stimulated B cells affects plasma cell markers. To determine if DP Bright also acts as a dominant negative protein in non-transformed B lymphocytes, splenic B cells were isolated by T cell depletion and stimulated with LPS for 20 hours prior to transduction with retroviral vectors producing DP Bright or with empty viral vector controls. Cells were sorted after 48 hrs on the basis of GFP production to isolate the transduced cells. Real time PCR for μ heavy chain transcripts was performed on mRNA isolated from transduced cells and data are presented in FIG. 15A. While one experiment showed an approximately 3-fold decrease in μ heavy chain transcripts in cells transduced with DP Bright-expressing virus compared to the control virus, three additional experiments showed no significant difference in μ heavy chain transcription.

While LPS stimulation is necessary to allow retroviral transduction of the B cells and to induce endogenous Bright expression (Webb et al., 1998), it also causes differentiation of B cells in culture including induction of isotype switching and deletion of the μ heavy chain locus. Therefore, the inventor examined other markers of plasma cell differentiation (Lin et al., 2002) for changes in response to expression of DP Bright. The surface proteins CD19 and CD138 change during plasma cell differentiation such that CD19 expression declines and CD138 expression increases. FIG. 15B shows surface expression profiles of transduced cells expressing DP Bright versus the control vector. Although the plasma cell marker CD138 was expressed equivalently in populations expressing the control and DP Bright, approximately twice as many cells transduced with DP Bright maintained CD19 expression as cells transduced with control vectors. IgM and IgD levels were comparable between control and DP transduced cells (not shown).

Examination of additional plasma cells differentiation markers by RT-PCR of mRNA purified form sorted transduced B cells demonstrated that J chain expression was induced equally well in cells expressing DP Bright and control transduced cells (FIG. 15C). Likewise, BLIMP and Pax-5 levels were also similar.

### Example 6

### Discussion

To gain an increased understanding of the function of the B cell-restricted transcription factor Bright, eight mutants were generated and analyzed. Mutations in the ARID domain resulted in reduced DNA binding activity, but did not affect formation of Bright dimers. Mutations of the conserved amino acid sequences, KIKK and REKLES, were insufficient to disrupt either DNA-binding activity or dimerization. Each mutant was able to interact with wild-type Bright to form heterodimers and several of the ARID mutations interfered with the DNA binding function of wild-type Bright. One of these proteins also interfered with the transcription activation potential of wild-type Bright in transfected cells expressing an immunoglobulin expression plasmid. These data suggest that ARID mutants can function as dominant negative proteins to interfere with Bright function. Finally, expression of dominant negative Bright in LPS-stimulated splenic B cells did not alter expression of many plasma cell differentiation markers, but affected expression of CD19.

Earlier homology analyses and deletion studies by Herrscher et al. (1995) suggested that both the ARID and a protein interaction domain were required for DNA-binding activity. Iwahara and Clubb (1999) solved the crystal structure of the ARID domain of Dri, the Drosophila homolog of Bright. NMR spectroscopy revealed that at least with Dri, the protein contacts DNA within the helix-loop-helix region of helices 5 and 6 (Iwahara et al., 2002). The mutation at P268 greatly decreased DNA binding activity. This mutation is within the β-sheet that is speculated to serve as a stabilizing factor in the protein-DNA interaction (Iwahara et al., 2002). W299 is in helix 5, F317 within helix 6 and Y330 is found in helix 7. None of these mutants bind DNA, confirming that helices 5 and 6 are important for DNA binding activity. In addition, these data also implicate specific residues in helix 7 are also important for Bright DNA binding activity. Thus, the studies reported here confirm the predicted importance of the ARID region and extend previous findings to demonstrate that even point mutations in important helices interfere with DNA-binding and function of the full-length protein.

Mixing studies using full-length Bright with deletion mutants suggested that Bright formed multiple complexes with its DNA-binding site leading others to propose that it binds to DNA as a tetramer (Herrscher et al., 1995). Deletion of the entire putative protein interaction domain abrogated DNA-binding in those studies. Size exclusion analyses demonstrate that Bright exists in nuclear extracts as a dimer. However, it cannot be ruled out that higher order complexes may be formed, particularly when Bright is bound to DNA. Indeed, others have proposed that Bright binds to multiple sites within the immunoglobulin heavy chain locus and forms DNA loops within that locus (Kaplan et al., 2001; Webb et al., 1999). Although these data do not bear upon this hypothesis, the co-precipitation of tagged Bright with wild-type Bright confirm the formation of dimeric Bright complexes and suggest that disruption of the REKLES region of the protein interaction domain is not sufficient to interfere with DNA-binding activity. Within the putative activation domain of mouse Bright is a region that is 90% homologous to a helix-turn-helix domain found in E2FBP1, human E2F binding protein, that was found to be necessary for interactions with E2F (Iwahara et al., 2002). The REKLES sequence is found within the first helix and indicates the possibility of the formation of previously unrecognized heteromeric complexes.

Bright, as a transcription factor, must be shuttled from the cytoplasm to the nucleus. Only proteins smaller than 40-45 kDa passively translocate to the nucleus through pore complexes (reviewed in Jans (1995); Miller (1991)). Bright is a 70 kDa protein, and must therefore contain a nuclear localization signal (NLS). NLSs are well known to contain basic regions as observed in the prototypic NLS of SV40 large T-antigen, PKKKRKV (SEQ ID NO:17) (Jans, 1995). Thus, the KIKK sequence in Bright acting as a NLS is generally consistent with other NLS consensus motifs.

Although several of the ARID mutants failed to bind DNA when expressed in non-B cells, only a few of those mutants inhibited binding of wild-type Bright to DNA. This presumably occurred due to the ability of the mutant proteins to form inactive heterodimers with wild-type Bright. Furthermore, the data presented here show that the DP Bright mutant also interferes with wild-type Bright function in a transcription assay using an immunoglobulin promoter reporter assay. Thus, this protein functions as a dominant negative protein.

Assessing the dominant negative activity of this mutant in B cells was more complicated. In a transfected B cell line that expressed constitutive Bright protein, no effect was observed unless the cells were first stimulated with LPS to induce enhanced levels of new endogenous Bright synthesis prior to transfection with dominant negative Bright. In this case, heterodimer formation of endogenous and tagged mutant Bright was possible. However, endogenous Bright dimers that were already present in the cell line did not appear to be affected by introduction of the dominant negative protein.

The majority of splenic B cells do not express Bright protein (Webb et al., 1998). Therefore, dominant negative Bright was introduced into those cells concomitantly with induction of native Bright. Retroviral transduction requires proliferating cells. LPS stimulation induces both B cell proliferation allowing transduction of dominant negative Bright into the cells and induction of endogenous Bright activity (Webb et al., 1998). However, LPS also induces plasma cell differentiation and isotype switching. Although no differences were observed in μ transcription, the expression of CD138 on the transduced cells clearly establishes that some of those cells differentiated into plasma cells even in the presence of dominant negative Bright. Therefore, they may also have undergone isotype switching and deletion of the μ locus, making it impossible to determine whether dominant negative Bright interfered with endogenous μ transcript expression. On the other hand, CD19 expression is down-regulated as B cells differentiate into plasma cells. B cells expressing dominant negative Bright exhibited approximately twice the number of cells expressing CD19 as those transduced with vector controls alone. Therefore, it is possible that expression of dominant negative Bright inhibited or slowed plasma cell differentiation in those cells. However, most of the cells expressing dominant negative Bright had lost CD19 expression. This result might be explained by incomplete inhibition of Bright activity in these cells, either through expression of inadequate dominant negative Bright levels or through early expression of stable wild-type Bright dimers before the transduced proteins are produced. On the other hand, several other plasma cell markers did not appear to be affected by dominant negative Bright expression. Therefore, inhibition of Bright activity may not be sufficient to affect plasma cell differentiation and Bright may not be important for late stage B cell differentiation.

Bright is also expressed in pre-B cells in the mouse (Webb et al., 1998), and its function in those cells is unknown. Although these data confirm the ability of Bright to transactivate an immunoglobulin reporter gene, definitive proof of Bright function in B cell differentiation awaits expression of dominant negative Bright in a transgenic model where dominant negative Bright can be expressed earlier than the endogenous wild-type B cell protein.

### Example 7

### Materials and Methods

Culture and Transient Transfections. The adherent Chinese Hamster Ovarian cell line (CHO) (ATCC, Manassas, Va.) was cultured in DMEM (Invitrogen, Carlsbad, Calif.) with 10% heat-inactivated fetal calf serum (FCS) (Atlanta Biologicals, Norcross, Ga.) and growth supplements as previously described (Webb et al., 2000). Transfections were performed using Fugene 6 transfection reagent as per the manufacturer's protocol (Roche, Indianapolis, Ind.). Briefly, 3 μg of each plasmid DNA and 30 μl of transfection reagent were incubated in serum free DMEM for 15 minutes and then added to a 60% confluent T-75 flask.

Constructs. The V1 reporter construct contained the S107 V1 heavy chain genomic sequence from −574 base pairs relative to the transcription start site to +146 base pairs and included the leader sequence, first intron and 146 bases of additional coding sequence (Webb et al., 1991). It was subcloned into pGEM-4Z (Invitrogen) with a 1 kb XbaI fragment containing the Eμ enhancer. Constructs containing deletions of the V1 5′-flanking sequence were produced as described (Webb et al., 1991). A full-length mouse Bright cDNA clone in pBKCMV (Herrscher et al., 1995) was used as a template to produce amino acid changes in the Bright coding sequence to produce a double point mutant (DPBr) with W299A and Y330A mutation that no longer binds DNA (Nixon et al., 2004). Wild-type and DPBr were tagged on the carboxyl terminus with the myc-his sequence using the vector pcDNA4/TO/myc-His B (Invitrogen). Briefly, Bright was PCR™ amplified with oligonucleotides that added 5′ EcoR I and 3′ Xba I sites while deleting the endogenous stop codon, and was ligated into these sites in the vector. Sequences were subcloned into the MiGR1 retroviral vector which expresses green fluoresecent protein (GFP) from an internal ribosomal entry site to allow visualization of transfected cells. The Btk-GFP fusion construct was a gift from Dr. William Rodgers (Oklahoma Medical Research Foundation, OMRF). The Btk wild-type, K430R and xid genes were amplified with Pfu polymerase (Stratagene, La Jolla, Calif.) from vaccinia viral clones (Fluckiger et al., 1998). The Btk PHTH deletion was generated by PCR™ mutagenesis removing the first 211 codons of the wild-type gene. All Btk constructs were subcloned into pcDNA4/HisMaxC (Invitrogen) and pET15b (Novagen, Madison, Wis.) for eukaryotic and prokaryotic expression, respectively. All constructs were sequenced by the OMRF Sequencing Core Facility to confirm their identities prior to use.

Real-Time Quantitative RT-PCR. Transfected cells were identified by GFP expression thirty-six hours post-transfection, and 200,000 GFP positive cells (of >95% purity) were collected for RNA isolation using a MoFlo Cell Sorter (Cytomation, Inc., Fort Collins, Colo.) by the OMRF or University of Oklahoma Health Sciences Center Flow Cytometry Cores. RNA was isolated using Tri-Reagent (Molecular Research Center, Inc., Cincinatti, Ohio) and immediately treated with DNaseI according to the manufacturer's protocol (Ambion, Austin, Tex.), extracted with phenol-chloroform (Clontec, Palo Alto, Calif.) and quantified by spectrophotometry. Reactions to generate cDNA included 300 ng RNA, 1 mM dNTP mix, 25 pM V1 specific primer or random primer (Integrated DNA Technologies, Coralville, Iowa), 40 units ribonuclease inhibitor RNasin (Promega, Madison, Wis.), and 200 units SuperScript™ II RNase-H Reverse Transcriptase (RT) (Invitrogen). Negative control reactions without RT were performed in parallel and baseline CT values of 36 to 40 were routinely obtained.

Real-Time quantitative RT-PCR was performed using specific TaqMan primers and probes to the V1 gene designed using PrimerExpress software (PE Applied Biosystems, Foster City, Calif.) and synthesized by Applied Biosystems and Integrated DNA Technologies: (forward: 5′-tgtcctgagttccccaatcc-3′ (SEQ ID NO:18); reverse: 5′-aaacccagtttaaccacatcttcat-3′ (SEQ ID NO:19); probe: 5′-6-FAM-acaattcagaaatcagcactcagtccttgtca-3′ (SEQ ID NO:20)). Reactions were performed in 1× TaqMan Universal PCR Master Mix (Applied Biosystems) in triplicate in 96 well plates with 250 nM of each primer and 500 nM probe using the following conditions: 50° C. for 2 minutes, 95° C. for 10 minutes, followed by 40 cycles at 95° C. for 15 seconds and 55° C. for 1 minute. A standard curve was generated by averaging CT values for triplicate reactions performed with 10-fold serial dilutions (from 20 to 10⁻⁴ ng) of plasmid containing the V1 gene. Three μl of cDNA was used as template to quantify V1 transcription and standards were run with every experiment and data were converted to ng. Data were analyzed using ABI Prism 7700 SDS analysis software (Applied Biosystems).

Western Blotting and Immunoprecipitation. Whole cell extracts were prepared from transfected CHO cells 36 hours post-transfection using hypotonic lysis as previously described (Buchanan et al., 1995). Briefly, cells were washed twice in PBS, suspended in extraction buffer (20 mM HEPES pH 7.9, 420 mM NaCl, 1 mM MgCl₂, 0.2 mM EDTA, 20% glycerol) containing 500 μM DTT and protease inhibitors (500 μM phenylmethylsulfonyl fluoride (PMSF), 20 μM leupeptin, 750 nM aprotinin and 30 mM NaVO₃), homogenized with pestles and incubated on ice for 30 minutes. Lysates were collected after 15 minutes of 4° C. centrifugation at 12,000 g and dialyzed at room temperature for 2 hours in storage buffer (20 mM HEPES pH 7.9. 100 mM KCl, 0.2 mM EDTA, 20% glycerol) containing protease inhibitors. Protein concentrations were measured by modified Bradford assay (BioRad, Hercules, Calif.) and western blotting was performed as previously described (Webb et al., 1993). Blots were developed with goat anti-Btk (C-20, Santa Cruz Biotechnology, Santa Cruz, Calif.), rabbit anti-Bright (gift from Dr. P. W. Tucker, U. Texas, Austin, Tex.), goat anti-BAP135/TFII-I peptide antibodies (prepared in this lab as previously described (Yang and Desiderio, 1997)) or mouse anti-phosphotyrosine, 4G10 (Upstate USA, Inc., Charlottesville, Va.) and AP-labeled rabbit anti-goat Ig, HRP-labeled anti-mouse IgG or AP-labeled anti-rabbit Ig (Southern Biotech, Birmingham, Ala.) as appropriate.

Lysates were immunoprecipitated with anti-Btk (C-20), anti-myc (Invitrogen) or control antibodies (anti-Sp1 or goat Ig) by rocking 150 μg of cell lysate with antibodies in PBS containing protease inhibitors at 4° C. for 2 hours. Protein A/G Plus-agarose beads (Santa Cruz) (25 μl; 1:1 slurry) were added and incubated at 4° C. for 12 hours. Immunoprecipitates were washed five times with wash buffer (100 mM Tris-Cl, 500 mM NaCl, 0.1% Tween 20, pH 8) containing protease inhibitors and proteins were eluted by addition of sodium dodecyl sulfate (SDS)-polyacrylamide gel electrophoresis sample buffer and boiling for 5 minutes. Samples were run on 8% SDS-polyacrylamide gels and transferred using standard protocols for western blotting as described above.

Electrophoretic Mobility Shift Assays (EMSA). Bright mobility shift assays were performed in 4% non-denaturing polyacrylamide gels as previously described (Buchanan et al., 1995). The prototypic Bright-binding site (a 150 bp BamHI fragment called bf150) from the V1 S107 5′ flanking sequence (Webb et al., 1991) labeled with α-³²P was used as probe. Bright protein was produced in vitro with TNT coupled rabbit reticulocyte lysates (Promega). Btk proteins were produced as recombinants in E. coli and then purified from inclusion bodies (Lin et al., 1994). Protein samples processed in parallel from E. coli lacking the Btk plasmid served as a negative control.

Antibody Facilitated DNA-Precipitation. BCg3R-1d cells stimulated for 20 hrs with 20 μg/ml LPS to induce Bright activity (Webb et al., 1989), or negative control T hybridoma cells, KD3B5.8 (gift of Dr. D. Farris, Oklahoma Medical Research Foundation), were subjected to crosslinking and immunoprecipitation according to Fernandez et al. (2001). Briefly, cells were subjected to crosslinking for 10 mins in 1% formaldehyde at 37° C., stopped with 0.125 M glycine for 5 min, and were then washed twice in cold PBS containing protease inhibitors (500 μM PMSF, 750 nM aprotinin and 20 μM leupeptin) before resuspension in SDS lysis buffer (1% SDS, 10 mM EDTA, 50 mM Tris pH 8) with protease inhibitors. After incubation on ice for 10 mins, the suspension was sonicated to reduce the DNA length to 200-1000 bp, centrifuged at 4° C. for 10 mins and the supernatant was used for immunoprecipitation. Sonicated extracts were precleared with Protein A/G beads (Santa Cruz) plus 50 μg salmon sperm DNA for 30 mins at 4° C. before incubation with either anti-Bright, anti-Btk or control goat Ig overnight at 4° C. Immunocomplexes were precipitated with blocked protein A/G beads for 1 hour. The beads were washed once in low salt buffer (0.1% SDS, 1% Triton X-100, 2 mM EDTA, 20 mM Tris, pH 8, 150 mM NaCl), once in high salt buffer (containing 500 mM NaCl), once in LiCl buffer (0.25M LiCl, 1% NP-40, 1% NaDOC, 1 mM EDTA, 10 mM Tris, pH 8) and twice in Tris-EDTA buffer. Immunocomplexes were eluted with 0.1 M NaHCO₃, 0.2% SDS and crosslinking was reversed by incubation at 65° C. for 4 hours. Ten μg proteinase K (Invitrogen) and 2 μl DNase-free RNase A (10 mg/ml) (Roche) were added for 2 hours at 50° C. The remaining immunoprecipitated DNA was phenol-chloroform purified and subjected to PCR using primers (5′-CTAGATCCACATGTATGATTT-3′ (SEQ ID NO:21), forward and 5′-GTCTTTCAGACAATAGATTGG-3′ (SEQ ID NO:22), reverse) that amplify a 150 bp Bright binding region of the V1 gene (Webb et al., 1991). PCR™ conditions used were 95° C. for 40 secs, 60° C. for 1 min, and 72° C. for 2 min for 40 cycles with a 10 min extension at 72° C.

### Example 8

### Results

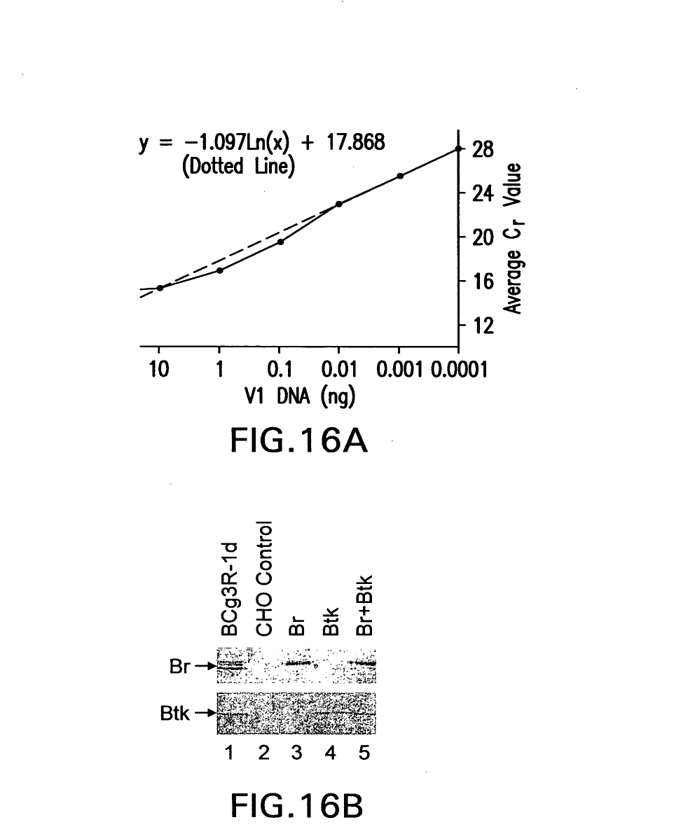

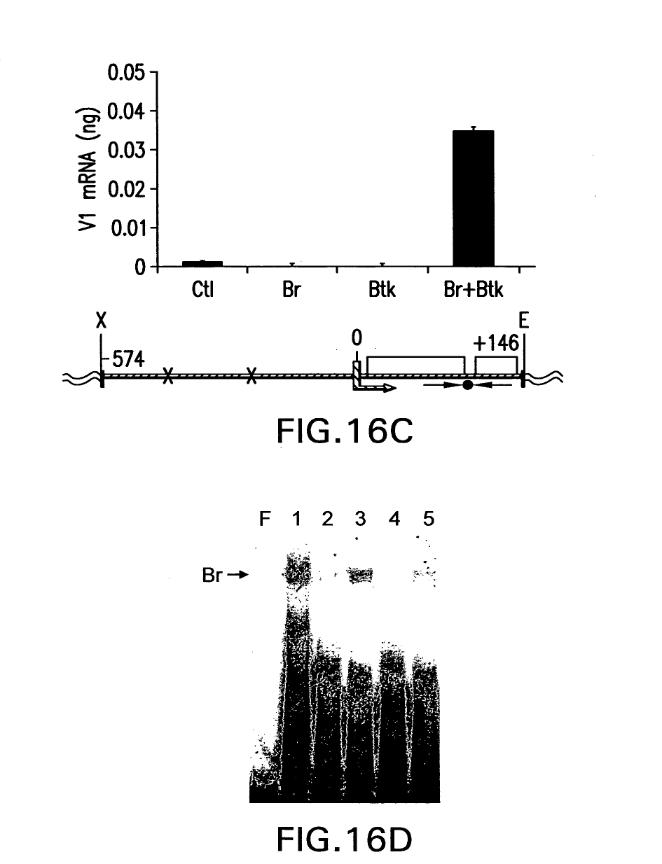

Btk is critically required for Bright activation of an immunoglobulin reporter gene. Previous studies demonstrated that both human and mouse Bright associate with Btk to produce DNA-binding complexes (Nixon et al., 2004; Webb et al., 2000). However, the functional relationship of the two proteins has not been shown. To determine whether Btk contributes to Bright's function as a transcription activator, Bright-induced transcription of an Ig promoter reporter construct was measured with and without Btk. The reporter construct contained two Bright binding sites previously shown to be necessary for upregulation of the V1 heavy chain gene in a B cell line (Webb et al., 1991). In addition, the native mouse V1 Ig heavy chain promoter extended through the leader, first intron and into the following exon of the V1 coding sequence (Buchanan et al., 1995) allowing PCR™ amplification across an intron. V1 Ig mRNA expression was quantified by Real-Time PCR. A standard curve was prepared using V1 cDNA (FIG. 16A). Amplification produced a single band in ethidium bromide stained gels and sequencing confirmed its identity. Serial dilutions of plasmid DNA containing the V1 gene were amplified in the presence of the Taqman Probe and CT values for 10⁻³ ng to 10⁻¹ ng of template DNA fell in the linear region of the curve.

To determine whether Btk influenced Bright activity, CHO cells that express neither Bright nor Btk were cotransfected with murine Bright and/or Btk expression vectors and the V1 reporter construct. Western blots showing protein expression of Bright and Btk in the transfected cells are shown in FIG. 16B. Control vectors containing the Bright coding sequence cloned in the reverse orientation, or the empty Btk vector, were used to maintain equal amounts of DNA in each transfection. Both the Bright and control vectors co-expressed GFP from an internal ribosomal entry site. To obtain cell populations containing equivalent numbers of transfected cells, GFP+ cells were isolated from each transfected population by cell sorting after 48 hours and mRNA was isolated for quantitative PCR (Q-PCR) analysis. Transfection efficiencies typically varied by less than 5% and the purity of sorted cells was >95%. In each case, the mean fluorescence intensity of the sorted cells was equivalent. Reactions lacking reverse transcriptase were run in parallel to ensure that the data produced resulted from amplification of the V1 cDNA rather than from the transfected vector and gave baseline CT values. FIG. 16C shows that cotransfection of Btk and Bright resulted in significantly increased V1 transcription in comparison with cells transfected with either Bright or Btk alone. Indeed, Bright and Btk singly transfected cells failed to show V1 transcription above control levels, while Bright and Btk increased transcription of the reporter construct from 3- to 12-fold. These findings show that Bright-dependent transcriptional activation in this cellular model is fully dependent upon co-expression of the Tec kinase, Btk.

The failure of Bright to transactivate V1 gene expression in the absence of Btk might result from the inability of Bright to bind DNA under those conditions. EMSA analyses of extracts from the transfectants described above were used to test the requirement of Btk for Bright DNA binding activity (FIG. 16D). Bright complexes were not formed in extracts that did not contain Bright. However, Bright bound to DNA in the absence of co-transfected Btk (lane 3). Therefore, Bright DNA-binding activity in transfected CHO cells does not require Btk. However, Bright transcriptional activity critically requires Btk.

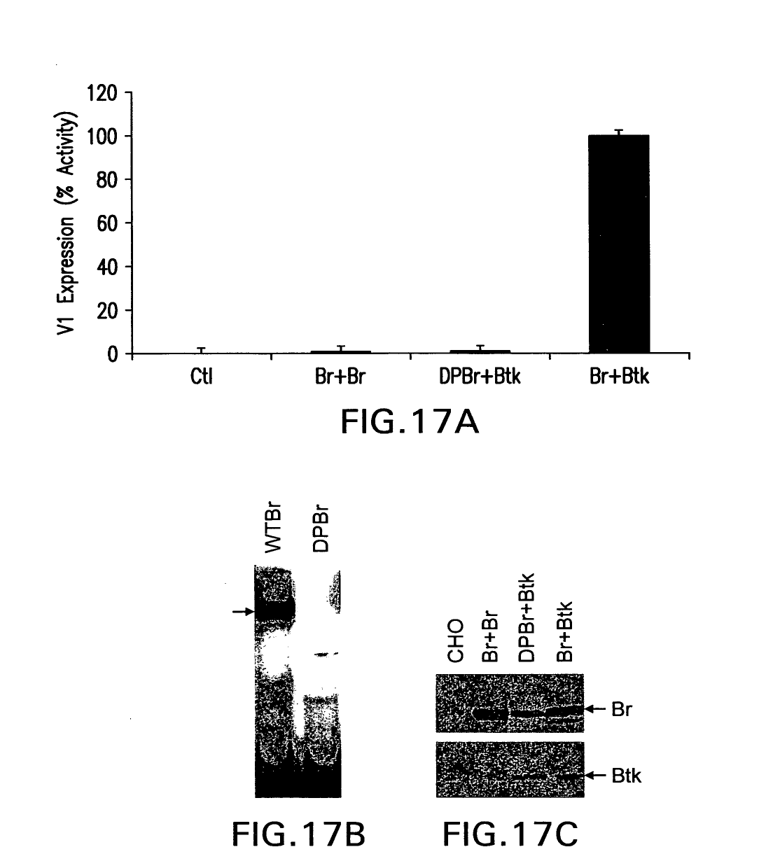

Bright DNA-binding activity is essential for transcription activation. To confirm that the DNA binding activity of Bright was required for V1 transcription, a mutant of Bright containing mutations W299A and Y330A resulting in a protein that failed to bind DNA (DPBr) (Nixon et al., 2004) was expressed with wild-type Btk in the CHO reporter system. Consistent with this model, V1 Ig transcripts were not generated in the presence of DPBr (FIGS. 17A-C). Mobility shift assays confirmed that DPBr did not bind DNA despite abundant protein expression (FIGS. 17B and 17C). In addition, because Bright binds DNA as a dimer, the inventor also asked if an increase in Bright expression levels might promote V1 transcription. Over-expression of Bright (up to 2-fold), however, failed to promote V1 transcription in the absence of co-expressed Btk (FIG. 17A). Therefore, Bright activity did not correlate with levels of Bright production in these cells. Rather, these data suggest that Bright DNA-binding activity is required for Bright function, but DNA-binding activity alone is not sufficient for transcriptional activity. Taken together, these findings suggested that Bright transcriptional activity was directly or indirectly regulated by Btk.

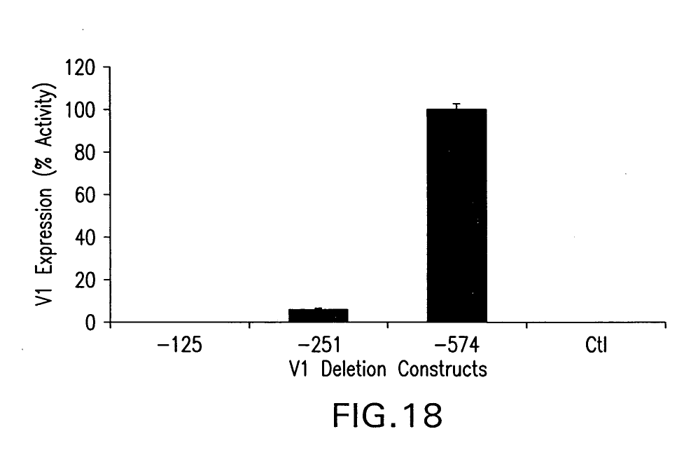

Bright activation of the V1 promoter is dependent on the 5′ Bright binding motif. The full length V1 promoter construct used in these studies (FIG. 17C) contains two Bright binding sites, one at approximately −550 and the other at −225 (Webb et al., 1991). Earlier analyses indicated that basal levels of V1 transcription in B lymphocytes required sequences from −125 to the transcription start site (Webb et al., 1991). These data were confirmed in V1 transgenic mice that expressed only 125 bases of the V1 promoter sequence (Avitahl and Calame, 1996). However, vectors containing the single Bright binding site at −225 resulted in a 3- to 6-fold enhancement of V1 transcription in B cell lines, and similar results were obtained with a −574 construct containing two Bright binding motifs (Webb et al., 1991). These previous studies utilized mature B cells. Transcription of the V1 promoter has not previously been possible in non-lymphocytic cell lines ((Herrscher et al., 1995) and inventor's unpublished data) probably due to a lack of Btk. To determine whether the transcriptional activation observed in the CHO transfectants required one or both of the Bright binding sites, the inventor compared transcription levels of V1 in CHO cells transfected with Btk plus Bright using either: a full length V1 construct with two Bright binding sites (−574); or with truncated V1 constructs containing one (−251) or no (−125) Bright binding sites. The previous data presented in FIG. 1 and FIG. 2 were obtained with the −574 construct where transcripts were quantified at 0.035 ng. This average value was therefore set as 100% activity (FIG. 18). The truncated vectors containing no or a single Bright binding site produced 0.0075% (−125) and 3.5% (−251) activity, respectively, in comparison with the full-length construct (FIG. 18). Negative control values (0.001%) were obtained with the full-length vector in the absence of either Bright or Btk proteins as described previously. These results suggest that the presence of the −550 Bright binding site is essential for transcriptional activity in this system.

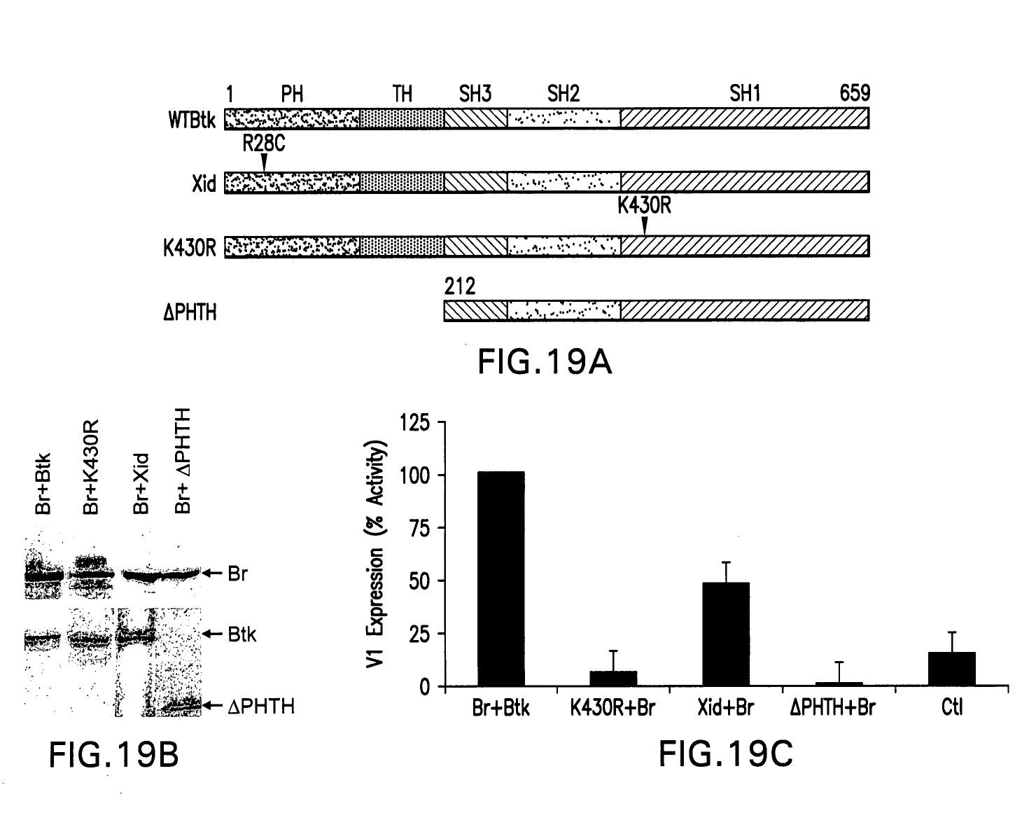

Bright function as a transcriptional activator requires Btk kinase activity and regions within the PH/TH domains of Btk. Mutated forms of Btk result in immunodeficiency disease in both mice and humans (Genevier and Callard, 1997; Rawlings et al., 1993; Thomas et al., 1993). To assess how inactive forms of Btk affect Bright activity, the inventor investigated whether the K430R (kinase inactive) (Li et al., 1997), xid (R28C) and pleckstrin and tec homology domains deletion (ΔPHTH) mutants of Btk affected V1 transcription levels (FIG. 19A). CHO cells were cotransfected with Bright, the full-length V1 reporter construct and with wild-type or mutant forms of Btk. Mutant and wild-type Btk protein expression in the transfectants was shown by western blotting (FIG. 19B). Transfectants expressing kinase inactive Btk and wild-type Bright exhibited <10% of the V1 transcription produced with wild-type Btk (FIG. 19C). Similarly, ΔPHTH Btk failed to enhance V1 transcription in this system. On the other hand, transfectants expressing the xid point mutation exhibited ˜50% of the V1 transcription observed with wild-type Btk. These data suggest that amino acid sequences within the PHTH domain are important for full activity. Moreover, the data indicate that Btk kinase activity is critically required for Bright transcription activation.

Btk is predominantly found in the cytoplasm of B cells, but we, and others, have observed Btk within the cell nucleus (Mohamed et al., 2000; Webb et al., 2000). A possible explanation for the failure of the Btk mutants to support Bright function (FIGS. 19A-C) is that either the mutant Btk proteins, or Bright, fail to enter the nucleus in those cells. To test this possibility, the intracellular localization and distribution of Bright and Btk was examined by confocal microscopy. Immunostaining of transfected CHO cells with anti-Bright and anti-Btk revealed both cytoplasmic and nuclear localization of both wild-type proteins (not shown). The K430R, xid and ΔPHTH mutations in Btk did not grossly alter the intracellular localization of Bright. Therefore, there is no major effect upon the localization of Bright in this model system that would explain the inability of the mutant forms of Btk to facilitate Bright-induced transcription.

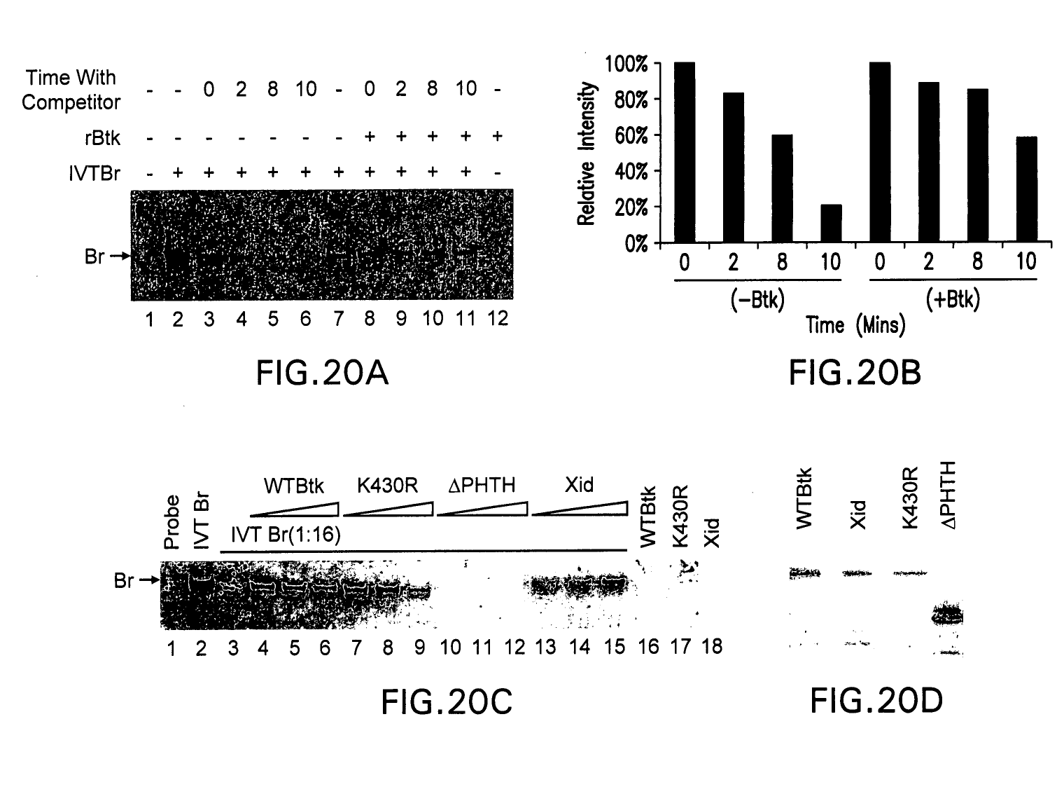

The PHTH domain of Btk is required for Btk-dependent enhancement of Bright DNA-binding activity. The inventor's earlier studies showed that addition of recombinant wild-type Btk to suboptimal levels of Bright protein enhanced Bright DNA-binding activity in mobility shift assays (Webb et al., 2000). To determine if Btk acts by stabilizing Bright binding activity, the inventor asked whether Bright binding affinity differed in the presence or absence of Btk. EMSAs were performed with in-vitro translated Bright and cold competitor DNA (FIG. 20A). In lanes 3-6 and 8-11, 100 molar excess of unlabeled competitor DNA was added for 0, 2, 8 and 10 minutes prior to electrophoresis. In lanes 8-12, recombinant Btk was also added to the proteins. Without Btk, Bright binding was reduced by 80% after 10 minutes with the competitor DNA (FIG. 20B). In the presence of Btk, Bright binding activity was reduced by less than 50% after 10 minutes with competitor DNA. These data suggest that one function of Btk is to enhance Bright DNA-binding affinity.

The K430R, ΔPHTH and xid forms of Btk failed to support V1 transcription activation by Bright. To investigate why these Btk mutants failed to support Bright function, the inventor asked if they enhanced Bright DNA-binding activity similarly to wild-type Btk. At a dilution of 1:16, no Bright DNA-binding activity was evident in in vitro translated extracts (FIG. 20C, lane 3). However, even low levels of recombinant wild-type Btk restored the DNA binding activity of Bright (lanes 4-6). The K430R and xid mutants were also effective in restoring Bright DNA-binding activity. Wild-type, K430R and xid Btk alone did not bind DNA without Bright (lanes 16-18). Relative protein levels of the Btk recombinant proteins used are shown in the right panel (FIG. 20C). On the other hand, Btk lacking the PHTH domains did not enhance Bright binding activity in this assay at any concentration of the mutant Btk protein (FIG. 20C, lanes 10-12). These data suggest that regions within the PHTH domains of Btk are required for facilitation of Bright DNA-binding activity by Btk.

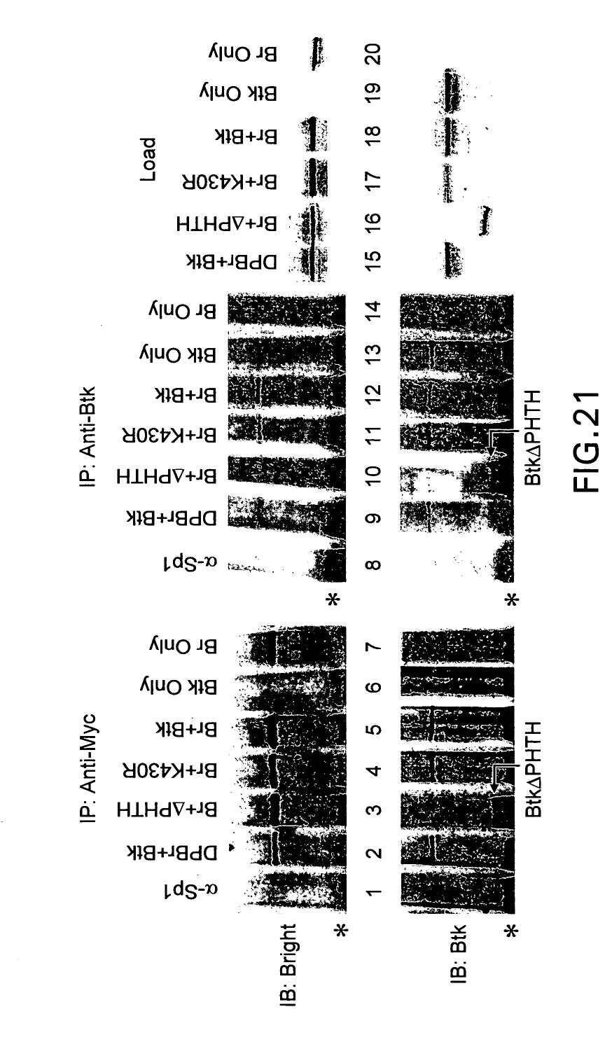

Bright associates with each of the Btk mutants examined. The inventor's earlier studies showed that Btk and Bright interact either directly or indirectly. To determine if the Btk mutants failed to facilitate Bright-induced transcription of the V1 gene because they failed to bind Bright, co-immunoprecipitation experiments were performed. Extracts from the CHO cells transfected with WTBr and Wt or mutant Btk were immunoprecipitated with antibodies to the myc tag on the carboxyl end of Bright. Precipitated proteins were detected with anti-Bright and anti-Btk (FIG. 21). While the isotype control (α-Sp1) did not precipitate Bright or Btk (lane 1), wild-type Bright coprecipitated Btk (lane 5). Anti-myc failed to precipitate Btk in the absence of Bright (lane 6). On the other hand, Btk coprecipitated with dominant negative Bright (lane 2) indicating that DPBr is still able to interact with Btk. This finding suggests that the absence of transcription activation observed with DPBr in FIGS. 17A-B was due to the mutant Bright's inability to bind DNA rather than failure to associate properly with Btk. The K430R mutant (lane 4) also precipitated with Bright, consistent with its ability to increase Bright affinity for DNA as shown in FIGS. 20A-C. Bright associated weakly with the ΔPHTH mutant (lane 3), although this Btk mutant did not increase Bright affinity for DNA. Likewise, in every case, immunoprecipitation of the Btk mutants with an antibody that recognized the SH1 domain also coprecipitated Bright (FIG. 21, lanes 9-12). These data show that Bright/Btk association is not disrupted by point mutations in the Bright DNA-binding domain or in the Btk kinase and PH domains. Furthermore, the PHTH domain is not required for Bright/Btk complex formation. These data suggest that the increased affinity of Bright for DNA is facilitated by the PHTH domain of Btk, but that additional regions of Btk, or associations with a third protein, account for its ability to associate with Bright.

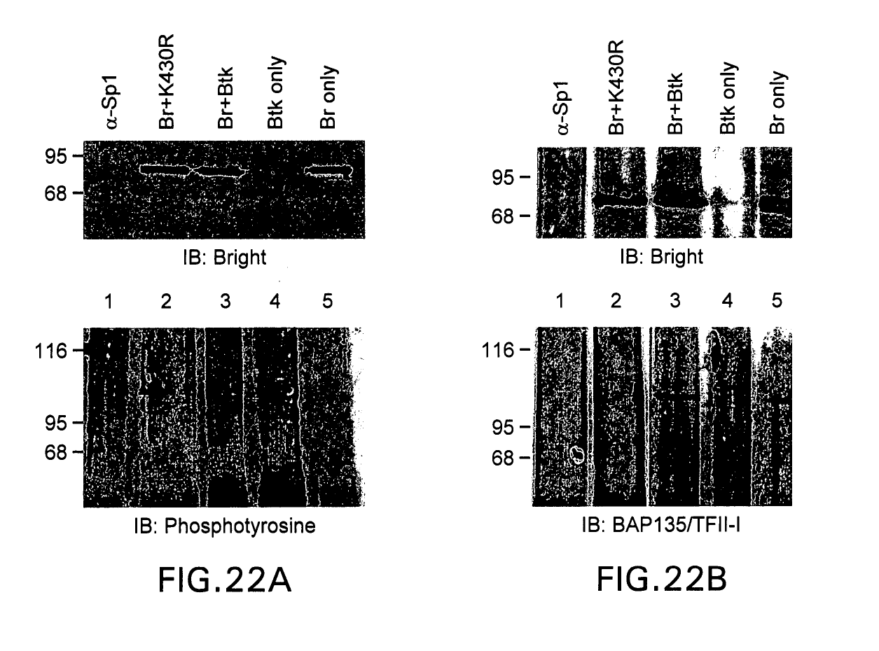

A Btk substrate associates with Bright. Although kinase inactive Btk co-precipitated with Bright (FIG. 21), it was not effective in enhancing immunoglobulin transcription in FIGS. 19A-B. The inventor's previous data suggested that Bright was not appreciably tyrosine phosphorylated (Webb et al., 2000). Therefore, the inventor hypothesized that a third protein, and Btk substrate, associates with Bright and Btk. CHO cells were transfected with myc-tagged Bright and/or Btk proteins, immunoprecipitated with anti-myc and immunoblotted for both Bright and phosphotyrosine (FIG. 22A). A 107 kDa band reactive with anti-phosphotyrosine co-precipitated with Bright in lane 3. A similar band, of weaker intensity was also evident in extracts that contained Bright without Btk (lane 5) and Bright with kinase inactive Btk (lane 2). No phosphorylated band was observed at 70 kDa, the expected size of phosphorylated Bright, in extracts that contained Bright. These data suggest that a third tyrosine-phosphorylated protein of 107 kDa co-precipitates with Bright. Although the intensity of the phosphorylated band was always greater in the extracts containing wild-type Btk and Bright, tyrosine phosphorylation of this protein is not solely dependent upon Btk.

A previously identified substrate of Btk in activated B cells is the transcription factor BAP135/TFII-I (Cheriyath et al., 1998; Yang and Desiderio, 1997). To narrow down the identity of the phosphorylated protein in FIG. 22A, similar immunoprecipitation experiments were performed and blots were developed with anti-BAP135/TFII-I (FIG. 22B). In each case, Bright co-precipitated from CHO cells with a 107 kDa protein that reacted with the anti-BAP/135 antibodies. These results are consistent with the hypothesis a third protein that is serologically related to BAP135/TFII-I is tyrosine phosphorylated by Btk and associates with Bright and Btk. Furthermore, these data suggest that Bright can associate with this protein in the absence of Btk.

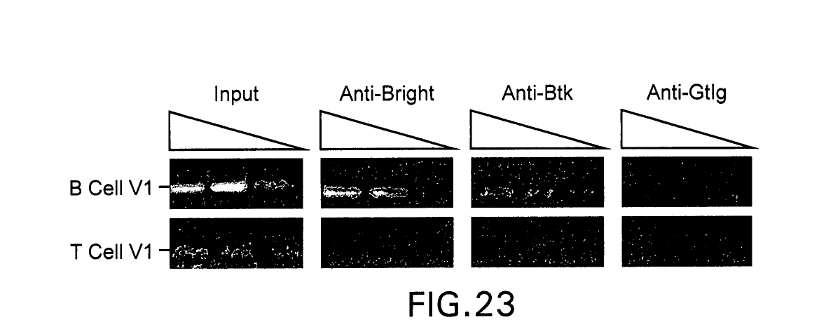

Bright/Btk complexes interact with a heavy chain promoter in a B cell line. Although the data from the previous experiments strongly suggest that Btk is required to enhance transcription of the V1 heavy chain, these data were all derived from artificially transfected non-B cells. To determine if Bright and Btk interact with the V1 promoter in B cells, the inventor took advantage of a B cell line that expresses a V1μ heavy chain gene, BCg3R-1d (Webb et al., 1989). Modified chromatin immunoprecipitation assays were conducted using anti-Bright, anti-Btk (C20) or control antibodies with LPS stimulated BCg3R-1d cells that express Bright (Webb et al., 1991; Webb et al., 1998) and a T cell hybridoma (KD3B5.8) that does not express either Bright or Btk. PCR™ primers were designed to amplify a region containing the Bright binding site between −574 and −425 of the V1 promoter (Webb et al., 1991). FIG. 23 shows amplification of the V1 Bright site using 10% of the input DNA obtained from either BCg3R-1d or KD3B5.8. Both anti-Bright and anti-Btk immunoprecipitated DNA from the B cell line contained the V1 Bright site, but neither antibody precipitated that region of DNA from the T cell line. In addition, control antibodies were unable to precipitate the V1 Bright binding site from either the B or T cell line. These data suggest that both Bright and Btk are present as a complex on the V1 promoter in the BCg3R1-d B cell line. Furthermore, these data are consistent with experiments in transfected CHO cells that suggest that Bright and Btk association facilitates V1 transcription.

### Example 9

### Discussion

The inventor's previous studies have suggested that Btk and Bright can associate to form a DNA-binding complex (Webb et al., 2000). However, it was not clear whether this association had any functional significance. In the present study, a novel model of Bright activity ws developed using a non-lymphoid cell line. Using this model, one clearly demonstrate that Bright-dependent transcription of an Ig promoter construct is fully dependent upon co-expressed, functional Btk. While Bright DNA-binding was required for its transcriptional activity, this was not sufficient to enhance Ig transcription. Furthermore, kinase inactive Btk did not facilitate Bright activated transcription in this system, suggesting that Btk kinase activity is critical for Bright function. Consistent with these findings, a third tyrosine phosphorylated protein was found to coprecipitate with Bright in CHO cells. This protein is serologically similar to the previously identified Btk substrate, BAP135/TFII-I. Moreover, modified ChIP experiments demonstrate that Bright/Btk complexes bind to Bright binding sites in the V1 promoter in a B cell line, consistent with a role for Bright/Btk complexes within B cells. These data are the first to demonstrate Bright activity in a non-B cell line and suggest that Btk provides additional B cell-specific information necessary for Ig heavy chain expression.

Although model systems such as this allow evaluation of the importance of protein/protein interactions through the use of ectopically expressed mutant proteins, the data obtained do not always faithfully reflect physiologic associations and expression levels. Antibody supershift experiments suggested that Bright and Btk form DNA-binding complexes in protein extracts from both mouse and human B cells (Nixon et al., 2004; Webb et al., 2000). Co-transfection of Bright and Btk in COS7 cells also gave results similar to those observed with the CHO cells (not shown). Furthermore, the inventor now presents evidence in modified ChIP analyses that Btk and Bright form a complex on the V1 promoter in the B cell line BCg3R-1d (FIG. 23). Therefore, it cannot be ruled out that some of the protein associations observed in the inventor's in vitro model system may partially reflect the abundant levels of the ectopically expressed proteins, or inappropriate intracellular localization of some of the proteins, multiple lines of evidence now support a role for functional Btk as a critical component of a Bright transcription complex.

The inventor's previous work (Webb et al., 2000), and FIGS. 22A-B, indicate that Bright is not appreciably phosphorylated on tyrosine residues and is therefore, unlikely to act as a direct substrate for Btk. The current data, however, clearly indicate a requirement for Btk kinase activity in Bright function. Together, these data suggest that an additional protein component(s), including a Btk kinase substrate, is likely to be required for Bright dependent transcriptional activity. TFII-I enhances transcription of promoters that lack TATA boxes and regulates promoter activity of the T cell receptor β locus through interactions with an initiator element (Cheriyath et al., 1998; Novina et al., 1998; Wu and Patterson, 1999). Although some IgH gene promoters contain good consensus TATA boxes, the V1 heavy chain promoter used in this system is TATA-less (Buchanan et al., 1997). Data from FIGS. 22A-B showed that a 107 kDa tyrosine phosphorylated protein co-immunoprecipitated with Bright in CHO cell extracts (FIGS. 22A-B). While this protein reacts serologically with antibodies against TFII-I, its precise identity is currently unknown. There are at least three gene families encoding TFII-I related proteins and individual members of these families are found in most cell types and can be expressed as multiple isoforms (Hinsley et al., 2004). Nothing is known regarding hamster TFII-I family proteins. Furthermore, cotransfection of kinase inactive Btk with Bright did not entirely eliminate the ability of the 107 kDa protein to react with anti-phophotyrosine antibodies (FIGS. 22A-B). Therefore, it cannot be concluded from these experiments that Btk phosphorylates the 107 kDa protein. TFII-I was shown previously to be phosphorylated by JAK2, ERK and Src, as well as by Btk (Cheriyath et al., 2002; Kim and Cochran, 2000; Kim and Cochran, 2001; Sacristán et al., 2004). In addition, residual phosphorylation of TFII-I without Btk has been observed by others (Sacristán et al., 2004). Both JAK2 and Btk can phosphorylate tyrosine 248 of TFII-I (Kim and Cochran, 2001; Sacristán et al., 2004). Additional studies will be required to determine if Bright/Btk complexes associate with TFII-I in B lymphocytes and if Bright activity requires tyrosine phosphorylation of the associated proteins. Nonetheless, results from the studies reported here are consistent with data from other labs in which the Btk-associated protein BAM11 exhibited increased transcription activity of a reporter construct ectopically expressed with Btk (Hirano et al., 2004; Kikuchi et al., 2000). In these studies, addition of TFII-I to the system further enhanced transcription activation (Hirano et al., 2004). Similary, the inventor's data are consistent with a model whereby Bright acts to tether Btk and a Btk substrate on the V1 immunoglobulin promoter such that transcription activation can occur. Transcription of the V1 promoter construct required binding of Bright to the Bright binding motifs. DP Bright that was unable to bind DNA was also ineffective at enhancing V1 transcription (FIGS. 17A-B), even though it associated with Btk (FIGS. 22A-B). Deletion of the Bright binding motifs in the V1 promoter also abrogated V1 transcription (FIG. 18). It is not clear why the presence of a single Bright binding site (−250 in the V1 construct) did not activate V1 transcription. However, the Bright binding motifs at −250 and −550 are not identical in sequence and only the −550 site was shown to be a matrix association region (Webb et al., 1991a; Webb et al., 1991b). Another possibility is that the intronic enhancer and its spacing relative to Bright are important for activity. Studies to address the function of the enhancer in this response are under way. In any case, data presented in FIGS. 20A-C suggest that Btk contributes to Bright DNA-binding activity by increasing its binding affinity. Together these data suggest a link between Bright DNA-binding activity and the requirement for fully functional Btk. The PH domain is a characteristic feature of the Tec family of tyrosine kinases. It is important for protein-protein interactions (Lowry et al., 2001) and for binding to phosphatidylinositol-3,4,5-bisphosphate (PIP₂) (Saito et al., 2003) and protein kinase C (PKC) in mast cells and B cells (Yao et al., 1994). In addition, the PHTH domain has been shown to be important for interaction with P15K, Vav, G proteins, F-actin, the tyrosine kinase FAK, phosphotyrosine phosphatase PTPD1, and the substrate for BCR downstream signaling 1 (BRDG1) (Qui and Kung, 2002; Saito et al., 2003; Satterthwaite and Witte, 2000; Yang et al., 2000). The inventor's data suggest that regions within the PH/TH domains are also important for Bright/Btk activity. When the PHTH deletion mutant of Btk was co-expressed with Bright, this mutant failed to upregulate V1 transcription. Furthermore, unlike the full-length forms of Btk, it failed to facilitate Bright DNA-binding activity in vitro. While these results could be explained by improper folding of the ΔPHTH proteins, immunoprecipitation experiments suggested that it associates weakly with Bright. However, the inventor's data now indicate that Bright interacts with a TFII-I related protein in CHO cells in the absence of Btk (FIG. 22B). Therefore, one cannot exclude the possibility that Btk interacts indirectly with Bright through its association with this third protein. Additional studies will be required to further elucidate these interactions.

A single amino acid change in the PH domain causes the xid defect in mice (Rawlings et al., 1993; Thomas et al., 1993). The xid mutant coprecipitated with Bright (not shown) and was capable of enhancing Bright DNA-binding activity when added to in vitro translated Bright. Moreover, the xid mutant cooperated with Bright to induce V1 transcription, although it was only half as efficient as wild-type Btk. This finding is consistent with the fact that the xid mutant retains kinase activity. Failure to activate transcription as efficiently as wild-type Btk may be due to conformational changes in the PH domain that affect the affinity of its association with Bright or a third TFII-I-related protein component required for V1 transcription. Others have reported that BAP135/TFII-I constitutively associates with wild-type Btk and kinase-inactive Btk, but not xid Btk (Novina et al., 1999). Intriguingly, xid mice fail to produce B cells that use the V1 heavy chain in response to immunization with phosphorylcholine, although the V1 gene is used predominantly in this response by both female littermates and other wild-type mice (Brown et al., 1985). The inventor's data suggest that the requirement for Btk for appropriate V1 expression may in part reflect its requirement for Bright-induced V1 promoter activity.

In the mouse, the xid mutation, or complete deficiency of Btk protein expression, causes blocks in B cell development at the immature B cell stage and results in impaired responses to Type II T-independent antigens, deficiencies in isotype switching to IgG₃ and low serum Ig production. In man, mutations in Btk generally result in less than 1% normal serum Ig levels caused by failure of the majority of B cells to differentiate beyond the pro-B cell stage (Nomura et al., 2000). Thus, the murine disease is less severe than the human defect. It is interesting to speculate that the ability of xid Btk to partially activate Ig transcription of the mouse V1 promoter might contribute to the presence of the immature B cells in the xid mouse.

Although Bright enhances heavy chain transcription 3- to 6-fold after B cell activation (Webb et al., 1991), it is not clear how this transcriptional upregulation contributes to B cell differentiation. Indeed, the role that Bright plays in B cell development remains incompletely defined. However, the fact that Bright function critically requires Btk kinase activity suggests that Bright could also be an important mediator of B cell differentiation. Other studies demonstrated the importance of Btk kinase activity for normal B cell development. In Btk deficient mice where the kinase inactive form of Btk was introduced as a transgene, the defective Btk rescued some of the defects associated with Btk deficiency, but not others (Middendorp et al., 2003). Regulation of Ig λ L chain usage was kinase independent and the modulation of pre-B cell marker expression was only partially dependent on kinase activity (Middendorp et al., 2003). Bright deficient mice will be required to determine if Bright, like Btk, is critically important for specific events in early or late B cell development.

### Example 10

Three transgenic mouse lines have been produced with varying levels of Bright transgene expression and have demonstrated that the transgene is expressed in early bone marrow B cell progenitors. Expression continues through into the mature stages at high levels, contrary to what is observed with endogenous Bright, where expression is limited to activated and pre-B cells. By sixteen weeks of age, all of the females tested have exhibited strong anti-nuclear antigen antibodies in the serum. The strain that expresses the highest level of Bright exhibited ANA in the serum at four weeks of age. ELISAs also demonstrated that anti-DNA antibodies were present in some of the mice, although autoimmune tendencies of this strain are being checked. In addition, several of the mice from the strain that expresses the highest levels of Bright also showed involuted thymuses with increased numbers of thymic B cells. This property has been observed in some autoimmune mice. Finally, staining of kidney sections from two mice with intermediate levels of Bright expression showed IgG in the glomeruli, also suggestive of autoimmunity.

### Example 11

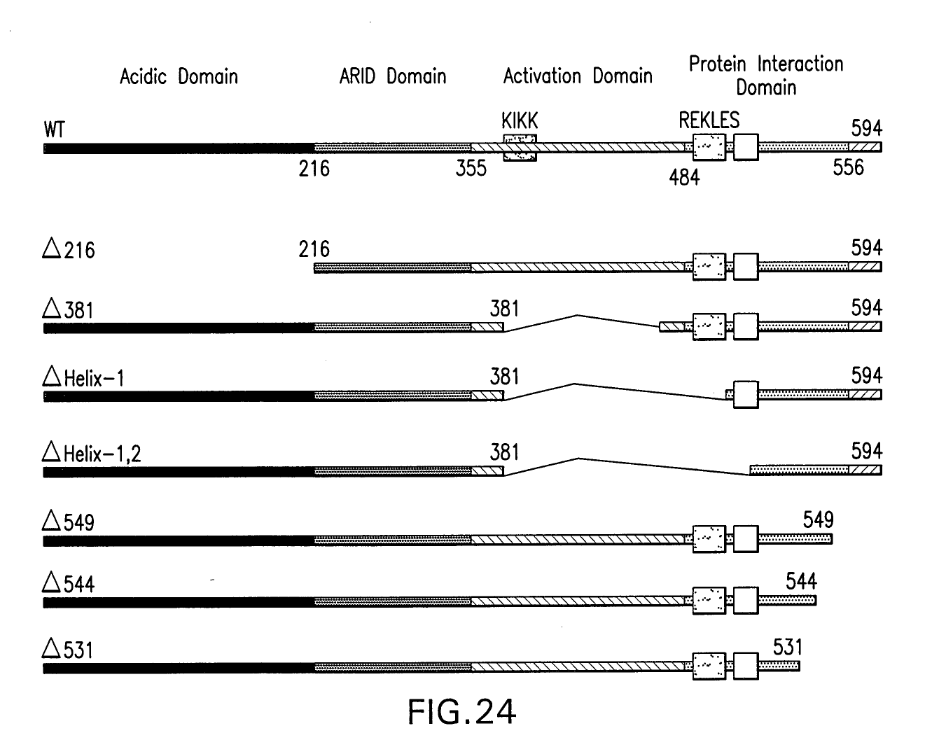

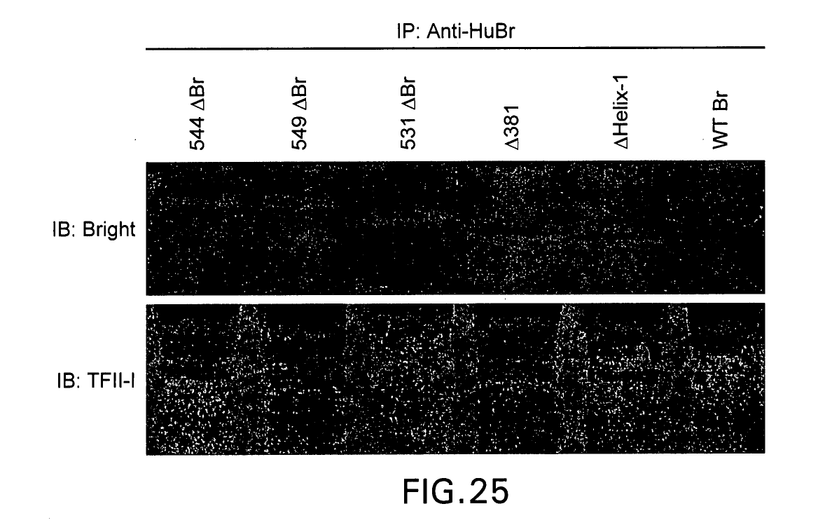

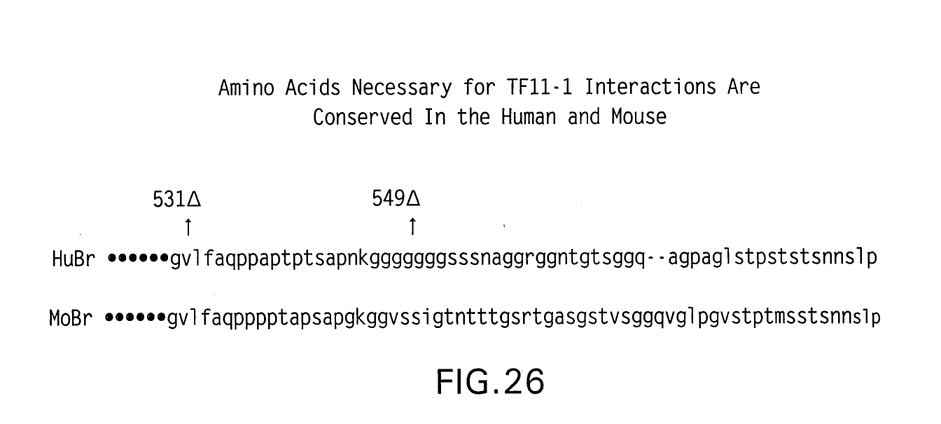

The inventors now have mutated human Bright and shown that the same amino acid sequence used to produce dominant-negative mouse Bright will also produce a dominant-negative human Bright (FIGS. 24 and 25). In addition, they have confirmed that TFII-I and human Bright interact, and do not require Btk for that interaction. Finally, they have identified a 19 amino acid peptide region of Bright that is required for interaction with TFII-I (FIGS. 25 and 26). Thus, the inventors' current model suggests that Bright recruits both TFII-I and Btk to Ig genes where Btk activates TFII-I which then upregulates Ig production. Therefore, they hypothesize that the small 19 amino acid peptide may also act as an inhibitor of Bright/TFII-I activity.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
U.S. Pat. No. 4,166,452
U.S. Pat. No. 4,196,265
U.S. Pat. No. 4,256,108
U.S. Pat. No. 4,265,874
U.S. Pat. No. 4,415,732
U.S. Pat. No. 4,458,066
U.S. Pat. No. 4,659,774
U.S. Pat. No. 4,682,195
U.S. Pat. No. 4,683,202
U.S. Pat. No. 4,816,571
U.S. Pat. No. 4,843,092
U.S. Pat. No. 4,946,778
U.S. Pat. No. 4,959,463
U.S. Pat. No. 5,141,813
U.S. Pat. No. 5,264,566
U.S. Pat. No. 5,354,855
U.S. Pat. No. 5,428,148
U.S. Pat. No. 5,554,744
U.S. Pat. No. 5,574,146
U.S. Pat. No. 5,602,244
U.S. Pat. No. 5,645,897
U.S. Pat. No. 5,705,629
U.S. Pat. No. 5,795,715
U.S. Pat. No. 5,888,773
U.S. Pat. No. 5,889,136
Alizadeh et al., Nature, 403:503-511, 2000.
Angel et al., Mol. Cell. Biol., 7:2256, 1987a.
Angel et al., Cell, 49:729, 1987b.
Arend and Dayer, Arthritis Rheum., 38:151-160, 1995.
Arend et al., Annu. Rev. Immunol., 16:27-55, 1998.
Atchison and Perry, Cell, 46:253, 1986.
Atchison and Perry, Cell, 48:121, 1987.
Avitahl and Calame, Int. Immunol., 8:1359-1366, 1996.
Baichwal and Sugden, In: Gene Transfer, Kucherlapati (Ed.), NY, Plenum Press, 117-148, 1986.
Banerji et al., Cell, 27(2 Pt 1):299-308, 1981.
Banerji et al., Cell, 33(3):729-740, 1983.
Barany and Merrifield, In: The Peptides, Gross and Meienhofer (Eds.), Academic Press, NY, 1-284, 1979.
Benvenisty and Neshif, Proc. Natl. Acad. Sci. USA, 83(24):9551-9555, 1986.
Berkhout et al., Cell, 59:273-282, 1989.
Blanar et al., EMBO J., 8:1139, 1989.
Bodine and Ley, EMBO J., 6:2997, 1987.
Boshart et al., Cell, 41:521, 1985.
Bosher et al., Nat. Cell. Biol., 2(2):E31-E36, 2000.
Bosze et al., EMBO J., 5(7):1615-1623, 1986.
Braddock et al., Cell, 58:269, 1989.
Brown et al., J. Immunol., 135:3558-3563, 1985.
Buchanan et al., J. Immunol., 155:4270-4277, 1995.
Buchanan et al., J. Immunol., 159:1247-1254, 1997.
Bulla and Siddiqui, J. Virol., 62:1437, 1986.
Burger and Dayer, Neurology, 45(6S-6):S39-43, 1995.
Burns and Peterson, Mol. Cell. Biol., 17:4811-4819, 1997.
Campbell and Villarreal, Mol. Cell. Biol., 8:1993, 1988.
Campbell et al., Am. Rev. Respir. Dis., 130(3):417-423, 1984.
Campere and Tilghman, Genes and Dev., 3:537, 1989.
Campo et al., Nature, 303:77, 1983.
Caplen et al., Gene, 252(1-2):95-105, 2000.
Celander and Haseltine, J. Virology, 61:269, 1987.
Celander et al., J. Virology, 62:1314, 1988.
Cerutti et al., J. Immunol., 160:2145-2157, 1998.
Chandler et al., Cell, 33:489, 1983.
Chang et al., Hepatology, 14:134A, 1991.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7(8):2745-2752, 1987.
Cheriyath et al., J. Biol. Chem., 277:22798-22805, 2002.
Cheriyath et al., Mol. Cell. Biol., 18:4444-4454, 1998.
Choi et al., Cell, 53:519, 1988.
Coffin, In: Virology, Fields et al. (Eds.), Raven Press, NY, 1437-1500, 1990.
Cohen et al., J. Cell. Physiol., 5:75, 1987.
Conley et al., Immunol. Rev., 138:5-21, 1994.
Cook et al., Cell, 27:487-496, 1981.
Costa et al., Mol. Cell. Biol., 8:81, 1988.
Couch et al., Am. Rev. Resp. Dis., 88:394-403, 1963.
Cripe et al., EMBO J., 6:3745, 1987.
Culotta and Hamer, Mol. Cell. Biol., 9:1376, 1989.
Dallas et al., Mol. Cell. Biol., 20:3137-3146, 2000.
Dandolo et al., J. Virology, 47:55-64, 1983.
De Villiers et al., Nature, 312(5991):242-246, 1984.
Deschamps et al., Science, 230:1174-1177, 1985.
Dinarello, Int. Rev. Immunol., 16:457-499, 1998.
Dubensky et al., Proc. Natl. Acad. Sci. USA, 81:7529-7533, 1984.
Eastgate et al., Lancet, 2:706-709, 1988.
Edbrooke et al., Mol. Cell. Biol., 9:1908, 1989.
Edlund et al., Science, 230:912-916, 1985.
Elbashir et al., Nature, 411(6836):494-498, 2001.
European App. EP 266 032
European Appln. EPO 0273085
Fattaey et al., Oncogene, 8:3149-3156, 1993.
Fechheimer, et al., Proc Natl. Acad. Sci. USA, 84:8463-8467, 1987.
Feng and Holland, Nature, 334:6178, 1988.
Ferkol et al., FASEB J., 7:1081-1091, 1993.
Fernandez et al., Mol. Cell Biol., 21:196-208, 2001.
Firak and Subramanian, Mol. Cell. Biol., 6:3667, 1986.
Fire et al., Nature, 391(6669):806-811, 1998.
Firestein et al., Arthritis Rheum., 37:644-652, 1994.
Fluckiger et al., EMBO J., 17:1973-1985, 1998.
Foecking and Hofstetter, Gene, 45(1):101-105, 1986.
Forster and Symons, Cell, 49(2):211-220, 1987.
Fraley et al., Proc. Natl. Acad. Sci. USA, 76:3348-3352, 1979.
Friedmann, Science, 244:1275-1281, 1989.
Froehler et al., Nucleic Acids Res., 14(13):5399-5407, 1986.
Fujikawa et al., Ann. Rheum. Dis., 54:318-320, 1995.
Fujita et al., Cell, 49:357, 1987.
Gefter, et al., Somatic Cell Genet., 3:231-236, 1977.
Genevier and Callard, Clin. Exp. Immunol., 110:386-391, 1997.
Gerlach et al., Nature (London), 328:802-805, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Goding, In: Monoclonal Antibodies: Principles and Practice, 2d ed., Academic Press, FL., 60-66, and 71-74, 1986.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Goodman and Gilman's The Pharmacological Basis Of Therapeutics, Hardman et al. (Eds.), 10^{th} Ed., 32:853-860; 35:891-893, 2001.
Gopal, Mol. Cell Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al, J. General Virology, 36:59-74, 1977.
Greene et al., Immunology Today, 10:272, 1989
Gregory et al., Mol. Cell. Biol. 16:792-799, 1996.
Grishok et al., Science, 287:2494-2497, 2000.
Grosschedl and Baltimore, Cell, 41:885, 1985.
Grunhaus and Horwitz, Seminar in Virology, 3:237-252, 1992.
Hahn and Tsao, In: Dubois' Lupus Erythematosus, 4^{th} Ed, Wallace and Hahn (Eds.), Lea and Febiger, Philadelphia, 195-201, 1993.
Hannum et al., Nature, 343:336-340, 1990.
Harland and Weintraub, J. Cell Biol., 101(3):1094-1099, 1985.
Harlow and Lane, In: Antibodies: A Laboratory Manual, Cold Spring Harbor Laboratory, Cold Spring Harbor, N.Y., 346-348, 1988.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Herrscher et al., Genes Dev. 9:3067-3082, 1995.
Hersdorffer et al., DNA Cell Biol., 9:713-723, 1990.
Herz and Gerard, Proc. Natl. Acad. Sci. USA, 90:2812-2816, 1993.
Hinsley et al., Protein Sci., 13:2588-2599, 2004.
Hirano, et al., Int. Immunol., 16:747-757, 2004.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Horwich et al. J. Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Inbra and Karin, Nature, 323:555, 1986.
Imler et al., Mol. Cell. Biol., 7:2558, 1987.
Imperiale and Nevins, Mol. Cell. Biol., 4:875, 1984.
Iwahara et al., EMBO J., 21:1197-1209, 2002.
Iwahara, and Clubb, EMBO J., 18:6084-6094, 1999.
Jakobovits et al., Mol. Cell. Biol., 8:2555, 1988.
Jameel and Siddiqui, Mol. Cell. Biol., 6:710, 1986.
Jans, Biochem. J., 311:705-716, 1995.
Jaynes et al., Mol. Cell. Biol., 8:62, 1988.
Johnson et al., Mol. Cell. Biol., 9:3393, 1989.
Jones and Shenk, Cell, 13:181-188, 1978.
Joyce et al., J. Biol. Chem., 274:25245-25249, 1999.
Kadesch and Berg, Mol. Cell. Biol., 6:2593, 1986.
Kahle et al., Ann. Rheum. Dis., 51:731-734, 1992.
Kaneda et al., Science, 243:375-378, 1989.
Kaplan et al., J. Biol. Chem., 276:21325-21330, 2001.
Karin et al., Mol. Cell. Biol., 7:606, 1987.
Karlsson et al., EMBO J., 5:2377-2385, 1986.
Katinka et al., Cell, 20:393, 1980.
Kato et al, J. Biol. Chem., 266:3361-3364, 1991.
Kawamoto et al., Mol. Cell. Biol., 8:267, 1988.
Ketting et al., Cell, 99(2):133-141, 1999.
Kikuchi et al., Int. Immunol., 12:1397-1408, 2000.
Kiledjian et al., Mol. Cell. Biol., 8:145, 1988.
Kim and Cochran, Mol. Cell. Biol., 20:1140-1148, 2000.
Kim and Cochran, Mol. Cell. Biol., 21:3387-3397, 2001.
Kim and Cook, Proc. Natl. Acad. Sci. USA, 84(24):8788-8792, 1987.
Klamut et al., Mol. Cell. Biol., 10:193, 1990.
Klein et al., Nature, 327:70-73, 1987.
Klein et al., Proc. Natl. Acad. Sci. USA, 100:2639-2644, 2003.
Koch et al., Mol. Cell. Biol., 9:303, 1989.
Kohler and Milstein, Eur. J. Immunol., 6:511-519, 1976.
Kohler and Milstein, Nature, 256:495-497, 1975.
Kortschak et al., Genomics, 51:288-292, 1998.
Kotzin and O'Dell, In: Samler's Immunologic Diseases, 5^{th} Ed., Frank et al. (Eds.), Little Brown & Co., Boston, 667-697, 1995.
Kotzin, Cell, 85:303-306, 1996.
Kouro et al., International Immunology, 13:485-493, 2001.
Krebs et al., Methods Cell Sci., 21:57-68, 1999.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Larsen et al., Proc Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Mol. Immunol., 39:923-932, 2003.
Lee et al., Nature, 294:228, 1981.
Lee et al., Nucleic Acids Res., 12:4191-206, 1984.
Levinson et al., Nature, 295:79, 1982.
Levrero et al., Gene, 101: 195-202, 1991.
Li et al., Oncogene, 15:1375-1383, 1997.
Lin and Grosschedl, Nature, 376:263-267, 1995.
Lin et al., Genetics, 153:1245-1256, 1999.
Lin et al., Methods Enzymol., 241:195-224, 1994.
Lin et al., Mol. Cell. Biol., 10:850, 1990.
Lin et al., Mol. Cell. Biol., 22:4771-4780, 2002.
Lipsky, In: Harrison's principles of internal medicine, Fauci et al. (Eds.), 14^{th} Ed., NY, McGraw-Hill, 1880-1888, 1998.
Lorch et al., Cell, 49:203, 1987.
Lowry et al., J. Biol. Chem., 276:45276-45281, 2001.
Lozzio et al., Proc. Soc. Exp. Biol. Med., 166:546-550, 1981.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Maas et al., J. Immunol., 162:6526-6553, 1999.
Macejak and Sarnow, Nature, 353:90-94, 1991.
Majors and Varmus, Proc. Natl. Acad. Sci. USA, 80:5866, 1983.
Mann et al., Cell, 33:153-159, 1983.
Markowitz et al., J. Virol., 62:1120-1124, 1988.
McNeall et al., Gene, 76:81, 1989.
Medina et al., Nat. Immunol., 2:718-724, 2001.
Merrifield, Science, 232(4748):341-347, 1986.
Michel and Westhof, J. Mol. Biol., 216:585-610, 1990.
Middendorp et al., J. Immunol., 168:2695-2703, 2002.
Middendorp et al., J. Immunol., 171:5988-5996, 2003.
Miksicek et al., Cell, 46:203, 1986.
Miller, Physiology Reviews, 71:909-949, 1991.
Mohamed et al., J. Biol. Chem., 275:40614-40619, 2000.
Montgomery et al., Proc. Natl. Acad. Sci. USA, 95:15502-15507, 1998.
Mordacq and Linzer, Genes and Dev., 3:760, 1989.
Moreau et al., Nucl. Acids Res., 9:6047, 1981.
Muesing et al., Cell, 48:691, 1987.
Ng et al., Nuc. Acids Res., 17:601, 1989.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nixon et al., Cellular Immunol., 228:42-53, 2004.
Nixon et al., J. Biol. Chem., 279(50):52465-52472, 2004.
Nomura et al., Blood, 96:610-617, 2000.
Novina et al., J. Biol. Chem., 273:33443-33448, 1998.
Novina et al., Mol. Cell. Biol., 19:5014-5024, 1999.
Numata et al., Cancer Res., 59:3741-3747, 1999.
Ohnishi et al., Int. Immunol., 6:817-830, 1994.
Ondek et al., EMBO J., 6:1017, 1987.
Ornitz et al., Mol. Cell. Biol., 7:3466, 1987.
Palmiter et al., Nature, 300:611, 1982.
Pascual et al., J. Exp. Med., 180:329-339, 1994.
Paskind et al., Virology, 67:242-248, 1975.
PCT Appln. WO 00/44914
PCT Appln. WO 01/68836
PCT Appln. WO 84/03564
PCT Appln. WO 99/32619
PCT Appln. WO 01/36646
Pear et al., Blood, 92:3780-3792, 1998.
Pear et al., Proc. Natl. Acad. Sci. USA, 90:8392-8396, 1993.
Pech et al., Mol. Cell. Biol., 9:396, 1989.
Pelletier and Sonenberg, Nature, 334(6180):320-325, 1988.
Perales et al., Proc. Natl. Acad. Sci. USA, 91:4086-4090, 1994.
Perez-Stable and Constantini, Mol. Cell. Biol., 10:1116, 1990.
Peterson and Herskowitz, Cell, 68:573-583, 1992.
Physicians Desk Reference,”
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10: 1076, 1990.
Potter et al., Proc. Natl. Acad. Sci. USA, 81:7161-7165, 1984.
Prieur et al., Lancet., 2:1240-1242, 1987.
Qiu and Kung, Oncogene, 19:5651-5661, 2000.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Rawlings et al., Science, 261:358-361, 1993.
Redondo et al., Science, 247:1225, 1990.
Reinhold-Hurek and Shub, Nature, 357:173-176, 1992.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Rodriguez and Denhardt (Eds.), Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Robertson et al., Nature 322:445-448, 1986.
Rooney et al., Rheumatol. Int., 10:217-219, 1990.
Rosen et al., Cell, 41:813, 1988.
Rosenfeld et al., Science, 252:431-434, 1991.
Rosenfeld, et al., Cell, 68:143-155, 1992.
Rossi et al., J. Immunol., 167:3033-3042, 2001.
Roux et al., Proc. Natl. Acad. Sci. USA, 86:9079-9083, 1989.
Sacristan et al., J. Biol. Chem., 279:7147-7158, 2004.
Saito et al., Immunity, 19:669-678, 2003.
Sakai et al., Genes and Dev., 2:1144, 1988.
Sambrook et al., In: Molecular cloning, Cold Spring Harbor Laboratory Press, Cold Spring Harbor, N.Y., 2001.
Sarver et al., Science, 247:1222-1225, 1990.
Satake et al., J. Virology, 62:970, 1988.
Satterthwaite and Witte, Ann. Rev. Immunol., 14:131-154, 1996.
Satterthwaite and Witte, Immunol. Rev., 175:120-127, 2000.
Scanlon et al., Proc. Natl. Acad. Sci. USA, 88:10591-10595, 1991.
Schaffner et al., J. Mol. Biol., 201:81, 1988.
Searle et al., Mol. Cell. Biol., 5:1480, 1985.
Sharp and Marciniak, Cell, 59:229, 1989.
Sharp et al., Science, 287:2431-2433, 2000.
Sharp, Genes Dev., 13:139-141, 1999.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stewart and Young, In: Solid Phase Peptide Synthesis, 2d. ed., Pierce Chemical Co., 1984.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Suzuki et al., Oncogene, 17:853-865, 1998.
Swartzendruber and Lehman, J. Cell. Physiology, 85:179, 1975.
Tabara et al., Cell, 99(2):123-132, 1999.
Takebe et al., Mol. Cell. Biol., 8:466, 1988.
Tam et al., J. Am. Chem. Soc., 105:6442, 1983.
Tavernier et al., Nature, 301:634, 1983.
Taylor and Kingston, Mol. Cell. Biol., 10:165, 1990a.
Taylor and Kingston, Mol. Cell. Biol., 10: 176, 1990b.
Taylor et al., J. Biol. Chem., 264:15160, 1989.
Temin, In: Gene Transfer, Kucherlapati (Ed.), NY, Plenum Press, 149-188, 1986.
The Merck Index, O'Neil et al., ed., 13^{th} ed., 2001.
Thiesen et al., J. Virology, 62:614, 1988.
Thomas et al., Science, 261:355-358, 1993.
Top et al., J. Infect. Dis., 124:155-160, 1971.
Treisman et al., Genes Dev. 11: 1949-1962, 1997.
Treisman, Cell, 42:889, 1985.
Tronche et al., Mol. Biol. Med., 7:173, 1990.
Trudel and Constantini, Genes and Dev. 6:954, 1987.
Tur-Kaspa et al., Mol. Cell Biol., 6:716-718, 1986.
Tyndell et al., Nuc. Acids. Res., 9:6231, 1981.
van den Berg, Semin. Arthritis Rheum., 30(5S-2):7-16, 2001.
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Wagner et al., Proc. Natl. Acad. Sci. USA 87(9):3410-3414, 1990.
Wang and Calame, Cell, 47:241, 1986.
Wang et al., Mol. Cell. Biol., 19:284-295, 1999.
Webb et al., Biochemistry, 28:4785-4790, 1989.
Webb et al., J. Immunol., 143:3934-3939, 1989.
Webb et al., Mol. Cell. Biol., 11:5206-5211, 1991.
Webb, et al., Mol. Cell. Biol., 11:5197-5205, 1991.
Webb et al., In: Differential regulation of immunoglobulin gene transcription via nuclear matrix-associated regions, Cold Spring Harbor Symp. Quant. Biol. LXIV, 109, 1999.
Webb et al., J. Immunol., 160:4747-4754, 1998.
Webb et al., J. Immunol., 165:6956-6965, 2000.
Webb et al., Nuc. Acids Res., 21:4363-4368, 1993.
Weber et al., Cell, 36:983, 1984.
Weinberger et al. Mol. Cell. Biol., 8:988, 1984.
Winoto and Baltimore, Cell 59:649, 1989.
Wong et al., Gene, 10: 87-94, 1980.
Wu and Patterson, J. Biol. Chem., 274:3207-3214, 1999.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yang and Desiderio, Proc. Natl. Acad. Sci. USA, 94:604-609, 1997.
Yang et al., Immunity, 12:373-382, 2000.
Yang et al., Proc. Natl. Acad. Sci. USA, 87:9568-9572, 1990.
Yao et al., Proc. Natl. Acad. Sci. USA, 91:9175-9179, 1994.
Yutzey et al. Mol. Cell. Biol., 9:1397, 1989.
Zelenin et al., FEBS Lett., 280:94-96, 1991.

1. A method of suppressing immunoglobulin production in an activated B cell comprising contacting said cell with an inhibitor of Bright polypeptide function.

2. The method of claim 1, wherein said inhibitor is an antisense molecule.

3. The method of claim 1, wherein said inhibitor is an interfering RNA.

4. The method of claim 1, wherein said inhibitor is a ribozyme.

5. The method of claim 1, wherein said inhibitor is a Bright-derived peptide.

6. The method of claim 5, wherein said peptide comprises at least a portion of a Bright dimerization domain.

7. The method of claim 5, wherein said peptide comprises at least a portion of a Bright DNA binding domain.

8. The method of claim 5, wherein said peptide comprises at least a portion of Btk-interacting domain.

9. The method of claim 1, wherein said inhibitor is dominant-negative Bright polypeptide.

10. The method of claim 1, wherein said inhibitor is an anti-Bright antibody or fragment thereof.

11. The method of claim 1, wherein said anti-Bright antibody or fragment thereof is an F′ab, an humanized antibody, or a single chain antibody.

12. The method of claim 1, wherein said inhibitor is delivered to said cell in a lipid delivery vehicle.

13. The method of claim 1, wherein said inhibitor is a polypeptide or a nucleic acid, and said inhibitor is delivered to said cell by an expression construct comprising an inhibitor coding region under the control of a promoter.

14. The method of claim 13, wherein said expression construct is a viral expression vector.

15. The method of claim 14, wherein said viral expression vector is an adenoviral vector, an adeno-associated viral vector, a retroviral vector, a vaccinia viral vector, a herpesviral vector or a polyoma viral vector.

16. The method of claim 15, wherein said viral expression vector is B cell tropic.

17. The method of claim 16, wherein said B cell tropic viral expression vector is Epstein Barr virus.

18. The method of claim 13, wherein said expression construct is a non-viral expression vector.

19. The method of claim 13, wherein said promoter is an inducible promoter and said method further comprises contacting said cell with an inducer of said promoter.

20. The method of claim 13, wherein said promoter is a B cell specific promoter.

21. The method of claim 13, wherein said promoter is a constitutive promoter.

22. A method of treating a subject afflicted with disease state associated with excessive immunoglobulin production comprising administering to said subject an inhibitor of Bright polypeptide function.

23. The method of claim 22, wherein said inhibitor is an antisense molecule.

24. The method of claim 22, wherein said inhibitor is an interfering RNA.

25. The method of claim 22, wherein said inhibitor is a ribozyme.

26. The method of claim 22, wherein said inhibitor is a Bright-derived peptide.

27. The method of claim 26, wherein said peptide comprises at least a portion of a Bright dimerization domain.

28. The method of claim 26, wherein said peptide comprises at least a portion of a Bright DNA binding domain.

29. The method of claim 26, wherein said peptide comprises at least a portion of Btk-interacting domain.

30. The method of claim 22, wherein said inhibitor is dominant-negative Bright polypeptide.

31. The method of claim 22, wherein said inhibitor is an anti-Bright antibody or fragment thereof.

32. The method of claim 22, wherein said anti-Bright antibody or fragment thereof is an F′ab, an humanized antibody, or a single chain antibody.

33. The method of claim 22, wherein said inhibitor is administered to said subject in a lipid delivery vehicle.

34. The method of claim 22, wherein said inhibitor is a polypeptide or a nucleic acid, and said inhibitor is administered to said subject via an expression construct comprising an inhibitor coding region under the control of a promoter.

35. The method of claim 34, wherein said expression construct is a viral expression vector.

36. The method of claim 35, wherein said viral expression vector is an adenoviral vector, an adeno-associated viral vector, a retroviral vector, a vaccinia viral vector, a herpesviral vector or a polyoma viral vector.

37. The method of claim 36, wherein said viral expression vector is B cell tropic.

38. The method of claim 37, wherein said B cell tropic viral expression vector is Epstein Barr virus.

39. The method of claim 34, wherein said expression construct is a non-viral expression vector.

40. The method of claim 34, wherein said promoter is an inducible promoter and said method further comprises contacting said cell with an inducer of said promoter.

41. The method of claim 34, wherein said promoter is a B cell specific promoter.

42. The method of claim 34, wherein said promoter is a constitutive promoter.

43. The method of claim 22, wherein said inhibitor is a peptide or a polypeptide which is fused to a TAT peptide.

44. The method of claim 22, further comprising administering to said subject an anti-inflammatory composition.

45. The method of claim 22, wherein said disease state is selected from the group consisting of systemic lupus erythematosus, rheumatoid arthritis, systemic sclerosis, polymyositis, Sjögren's Syndrome, graft rejection, Grave's disease, myasthenia gravis, cancer characterized by hyperimmunoglobulinemia, mononucleosis, and a hyper-Ig syndrome.

46. The method of claim 22, wherein said inhibitor is administered more than once.

47. The method of claim 22, wherein said inhibitor is administered on a chronic basis.

48. A method of screening for a suppressor of immunoglobulin production comprising
(a) providing at least two Bright polypeptides;
(b) contacting said Bright polypeptides with a candidate substance; and
(c) assessing Bright dimer formation,
wherein a decrease in Bright dimer formation, as compared to Bright dimer formation observed in the absence of said candidate substance, identifies said candidate substance as a suppressor of immunoglobulin production.

49. The method of claim 48, wherein said candidate substance is a peptide, a non-functional Bright analog, an antibody or antibody fragment, or a small molecule organopharmaceutical.

50. A method of screening for a suppressor of immunoglobulin production comprising:
(a) providing at least one Bright polypeptide and one Btk polypeptide;
(b) contacting said polypeptides with a candidate substance; and
(c) assessing Bright interaction with Btk,
wherein a decrease in Bright interaction with Btk, as compared to Bright interaction with Btk observed in the absence of said candidate substance, identifies said candidate substance as a suppressor of immunoglobulin production.

51. The method of claim 50, wherein said candidate substance is a peptide, a non-functional Bright analog, an antibody or antibody fragment, or a small molecule organopharmaceutical.

52. A method of screening for a suppressor of immunoglobulin production comprising:
(a) providing at least one Bright polypeptide and one TFII-I polypeptide;
(b) contacting said polypeptides with a candidate substance; and
(c) assessing Bright interaction with TFII-I,
wherein a decrease in Bright interaction with TFII-I, as compared to Bright interaction with TFII-I observed in the absence of said candidate substance, identifies said candidate substance as a suppressor of immunoglobulin production.

53. The method of claim 52, wherein said candidate substance is a peptide, a non-functional Bright analog, an antibody or antibody fragment, or a small molecule organopharmaceutical.

54. A method of screening for a suppressor of immunoglobulin production comprising:
(a) providing a recombinant cell that expresses Bright polypeptide and Btk polypeptide, and further comprises an immunoglobulin promoter linked to a screenable or selectable marker;
(b) contacting said cell with a candidate substance; and
(c) assessing expression of said marker,
wherein a decrease in expression of said marker, as compared to marker expression observed in the absence of said candidate substance, identifies said candidate substance as a suppressor of immunoglobulin production.

55. The method of claim 54, wherein said recombinant cell further expresses TFII-I.
